SEQUENCE LISTING (11 sequences)

SEQ ID NO: 1 - DNA, 5116 bp, Haemophilus influenzae
acagcgttctcttaatactagtacaaacccacaataaaatatgacaaacaacaattacaacaccttttttgcagtctatatgcaaatattttaaaaaatagtataaatccgccatataaaatggtataatctttcatctttcatctttcatctttcatctttcatctttcatctttcatctttcatctttcatctttcatctttcatctttcatctttcatctttcatctttcatctttcacatgccctgatgaaccgagggaagggagggaggggcaagaatgaagagggagctgaacgaacgcaaatgataaagtaatttaattgttcaactaaccttaggagaaaatatgaacaagctatatcgtctcaaattcagcaaacgcctgaatgctttggttgctgtgtctgaattggcacggggttgtgaccattccacagaaaaaggcagcgaaaaacctgctcgcatgaaagtgcgtcacttagcgttaaagccactttccgctatgttactatctttaggtgtaacatctattccacaatctgttttagcaagcggcttacaaggaatggatgtagtacacggcacagccactatgcaagtagatggtaataaaaccattatccgcaacagtgttgacgatatcattaattggaaacaatttaacatcgaccaaaatgaaatggtgcagtttttacaagaaaacaacaactccgccgtattcaaccgtgttacatctaaccaaatctcccaattaaaagggattttagattctaacggacaagtctttttaatcaacccaaatggtatcacaataggtaaagacgcaattattaacactaatggctttacggcttctacgctagacatttctaacgaaaacatcaaggcgcgtaatttcaccttcgagcaaaccaaagataaagcgctcgctgaaattgtgaatcacggtttaattactgtcggtaaagacggcagtgtaaatcttattggtggcaaagtgaaaaacgagggtgtgattagcgtaaatggtggcagcatttctttactcgcagggcaaaaaatcaccatcagcgatataataaacccaaccattacttacagcattgccgcgcctgaaaatgaagcggtcaatctgggcgatatttttgccaaaggcggtaacattaatgtccgtgctgccactattcgaaaccaaggtaaactttctgctgattctgtaagcaaagataaaagcggcaatattgttctttccgccaaagagggtgaagcggaaattggcggtgtaatttccgctcaaaatcagcaagctaaaggcggcaagctgatgattacaggcgataaagtcacattaaaaacaggtgcagttatcgacctttcaggtaaagaagggggagaaacttaccttggcggtgacgagcgcggcgaaggtaaaaagggcattcaattagcaaagaaaacctctttagaaaaaggctcaaccatcaatgtatcaggcaaagaaaaaggcggacgcgctattgtgtggggcgatattgcgttaattgacggcaatattaacgctcaaggtagtggtgatatcgctaaaaccggtggttttgtggagacgtcggggcatgatttattcatcaaagacaatgcaattgttgacgccaaagagtggttgttagacccggataatgtatctattaatgcagaaacagcaggacgcagcaatacttcagaagacgatgaatacacgggatccgggaatagtgccagcaccccaaaacgaaacaaagaaaagacaacattaacaaacacaactcttgagagtatactaaaaaaaggtacctttgttaacatcactgctaatcaacgcatctatgtcaatagctccattaatttatccaatggcagcttaactctttggagtgagggtcggagcggtggcggcgttgagattaacaacgatattaccaccggtgatgataccagaggtgcaaacttaacaatttactcaggcggctgggttgatgttcataaaaatatctcactcggggcgcaaggtaacataaacattacagctaaacaagatatcgcctttgagaaaggaagcaaccaagtcattacaggtcaagggactattacctcaggcaatcaaaaaggttttagatttaataatgtctctctaaacggcactggcagcggactgcaattcaccactaaaagaaccaataaatacgctatcacaaataaatttgaagggactttaaatatttcagggaaagtgaacatctcaatggttttacctaaaaatgaaagtggatatgataaattcaaaggacgcacttactggaatttaacctccttaaatgtttccgagagtggcgagtttaacctcactattgactccagaggaagcgatagtgcaggcacacttacccagccttataatttaaacggtatatcattcaacaaagacactacctttaatgttgaacgaaatgcaagagtcaactttgacatcaaggcaccaatagggataaataagtattctagtttgaattacgcatcatttaatggaaacatttcagtttcgggaggggggagtgttgatttcacacttctcgcctcatcctctaacgtccaaacccccggtgtagttataaattctaaatactttaatgtttcaacagggtcaagtttaagatttaaaacttcaggctcaacaaaaactggcttctcaatagagaaagatttaactttaaatgccaccggaggcaacataacacttttgcaagttgaaggcaccgatggaatgattggtaaaggcattgtagccaaaaaaaacataacctttgaaggaggtaacatcacctttggctccaggaaagccgtaacagaaatcgaaggcaatgttactatcaataacaacgctaacgtcactcttatcggttcggattttgacaaccatcaaaaacctttaactattaaaaaagatgtcatcattaatagcggcaaccttaccgctggaggcaatattgtcaatatagccggaaatcttaccgttgaaagtaacgctaatttcaaagctatcacaaatttcacttttaatgtaggcggcttgtttgacaacaaaggcaattcaaatatttccattgccaaaggaggggctcgctttaaagacattgataattccaagaatttaagcatcaccaccaactccagctccacttaccgcactattataagcggcaatataaccaataaaaacggtgatttaaatattacgaacgaaggtagtgatactgaaatgcaaattggcggcgatgtctcgcaaaaagaaggtaatctcacgatttcttctgacaaaatcaatattaccaaacagataacaatcaaggcaggtgttgatggggagaattccgattcagacgcgacaaacaatgccaatctaaccattaaaaccaaagaattgaaattaacgcaagacctaaatatttcaggtttcaataaagcagagattacagctaaagatggtagtgatttaactattggtaacaccaatagtgctgatggtactaatgccaaaaaagtaacctttaaccaggttaaagattcaaaaatctctgctgacggtcacaaggtgacactacacagcaaagtggaaacatccggtagtaataacaacactgaagatagcagtgacaataatgccggcttaactatcgatgcaaaaaatgtaacagtaaacaacaatattacttctcacaaagcagtgagcatctctgcgacaagtggagaaattaccactaaaacaggtacaaccattaacgcaaccactggtaacgtggagataaccgctcaaacaggtagtatcctaggtggaattgagtccagctctggctctgtaacacttactgcaaccgagggcgctcttgctgtaagcaatatttcgggcaacaccgttactgttactgcaaatagcggtgcattaaccactttggcaggctctacaattaaaggaaccgagagtgtaaccacttcaagtcaatcaggcgatatcggcggtacgatttctggtggcacagtagaggttaaagcaaccgaaagtttaaccactcaatccaattcaaaaattaaagcaacaacaggcgaggctaacgtaacaagtgcaacaggtacaattggtggtacgatttccggtaatacggtaaatgttacggcaaacgctggcgatttaacagttgggaatggcgcagaaattaatgcgacagaaggagctgcaaccttaactacatcatcgggcaaattaactaccgaagctagttcacacattacttcagccaagggtcaggtaaatctttcagctcaggatggtagcgttgcaggaagtattaatgccgccaatgtgacactaaatactacaggcactttaactaccgtgaagggttcaaacattaatgcaaccagcggtaccttggttattaacgcaaaagacgctgagctaaatggcgcagcattgggtaaccacacagtggtaaatgcaaccaacgcaaatggctccggcagcgtaatcgcgacaacctcaagcagagtgaacatcactggggatttaatcacaataaatggattaaatatcatttcaaaaaacggtataaacaccgtactgttaaaaggcgttaaaattgatgtgaaatacattcaaccgggtatagcaagcgtagatgaagtaattgaagcgaaacgcatccttgagaaggtaaaagatttatctgatgaagaaagagaagcgttagctaaacttggagtaagtgctgtacgttttattgagccaaataatacaattacagtcgatacacaaaatgaatttgcaaccagaccattaagtcgaatagtgatttctgaaggcagggcgtgtttctcaaacagtgatggcgcgacggtgtgcgttaatatcgctgataacgggcggtagcggtcagtaattgacaaggtagatttcatcctgcaatgaagtcattttattttcgtattatttactgtgtgggttaaagttcagtacgggctttacccatcttgtaaaaaattacggagaatacaataaagtatttttaacaggttattattatg

SEQ ID NO: 2 - protein, 1536 aa, Haemophilus influenzae
MNKIYRLKFSKRLNALVAVSELARGCDHSTEKGSEKPARMKVRHLALKPLSAMLLSLGVTSIPQSVLASGLQGMDVVHGTATMQVDGNKTIIRNSVDAIINWKQFNIDQNEMVQFLQENNNSAVFNRVTSNQISQLKGILDSNGQVFLINPNGITIGKDAIINTNGFTASTLDISNENIKARNFTFEQTKDKALAEIVNHGLITVGKDGSVNLIGGKVKNEGVISVNGGSISLLAGQKITISDIINPTITYSIAAPENEAVNLGDIFAKGGNINVRAATIRNQGKLSADSVSKDKSGNIVLSAKEGEAEIGGVISAQNQQAKGGKLMITGDKVTLKTGAVIDLSGKEGGETYLGGDERGEGKNGIQLAKKTSLEKGSTINVSGKEKGGRAIVWGDIALIDGNINAQGSGDIAKTGGFVETSGHDLFIKDNAIVDAKEWLLDFDNVSINAETAGRSNTSEDDEYTGSGNSASTPKRNKEKTTLTNTTLESILKKGTFVNITANQRIYVNSSINLSNGSLTLWSEGRSGGGVEINNDITTGDDTRGANLTIYSGGWVDVHKNISLGAQGNINITAKQDIAFEKGSNQVITGQGTITSGNQKGFRFNNVSLNGTGSGLQFTTKRTNKYAITNKFEGTLNISGKVNISMVLPKNESGYDKFKGRTYWNLTSLNVSESGEFNLTIDSRGSDSAGTLTQPYNLNGISFNKDTTFNVERNARVNFDIKAPIGINKYSSLNYASFNGNISVSGGGSVDFTLLASSSNVQTPGVVINSKYFNVSTGSSLRFKTSGSTKTGFSIEKDLTLNATGGNITLLQVEGTDGMIGKGIVAKKNITFEGGNITFGSRKAVTEIEGNVTINNNANVTLIGSDFDNHQKPLTIKKDVIINSGNLTAGGNIVNIAGNLTVESNANFKAITNFTFNVGGLFDNKGNSNISIAKGGARFKDIDNSKNLSITTNSSSTYRTIISGNITNKNGDLNITNEGSDTEMQIGGDVSQKEGNLTISSDKINITKQITIKAGVDGENSDSDATNNANLTIKTKELKLTQDLNISGFNKAEITAKDGSDLTIGNTNSADGTNAKKVTFNQVKDSKISADGHKVTLHSKVETSGSNNNTEDSSDNNAGLTIDAKNVTVNNNITSHKAVSISATSGEITTKTGTTINATTGNVEITAQTGSILGGIESSSGSVTLTATEGALAVSNISGNTVTVTANSGALTTLAGSTIKGTESVTTSSQSGDIGGTISGGTVEVKATESLTTQSNSKIKATTGEANVTSATGTIGGTISGNTVNVTANAGDLTVGNGAEINATEGAATLTTSSGKLTTEASSHITSAKGQVNLSAQDGSVAGSINAANVTLNTTGTLTTVKGSNINATSGTLVINAKDAELNGAALGNHTVVNATNANGSGSVIATTSSRVNITGDLITINGLNIISKNGINTVLLKGVKIDVKYIQPGIASVDEVIEAKRILEKVKDLSDEEREALAKLGVSAVRFIEPNNTITVDTQNEFATRPLSRIVISEGRACFSNSDGATVCVNIADNGR

SEQ ID NO: 3 - DNA, 4937 bp, Haemophilus influenzae
taaatatacaagataataaaaataaatcaagatttttgtgatgacaaacaacaattacaacaccttttttgcagtctatatgcaaatattttaaaaaaatagtataaatccgccatataaaatggtataatctttcatctttcatctttaatctttcatctttcatctttcatctttcatctttcatctttcatctttcatctttcatctttcatctttcatctttcatctttcatctttcacatgaaatgatgaaccgagggaagggagggaggggcaagaatgaagagggagctgaacgaacgcaaatgataaagtaatttaattgttcaactaaccttaggagaaaatatgaacaagatatatcgtctcaaattcagcaaacgcctgaatgctttggttgctgtgtctgaattggcacggggttgtgaccattccacagaaaaaggcttccgctatgttactatctttaggtgtaaccacttagcgttaaagccactttccgctatgttactatctttaggtgtaacatctattccacaatctgttttagcaagcggcttacaaggaatggatgtagtacacggcacagccactatgcaagtagatggtaataaaaccattatccgcaacagtgttgacgctatcattaattggaaacaatttaacatcgaccaaaatgaaatggtgcagtttttacaagaaaacaacaactccgccgtattcaaccgtgttacatctaaccaaatctcccaattaaaagggattttagattctaacggacaagtctttttaatcaacccaaatggtatcacaataggtaaagacgcaattattaacactaatggctttacggcttctacgctagacatttctaacgaaaacatcaaggcgcgtaatttcaccttcgagcaaaccaaagataaagcgctcgctgaaattgtgaatcacggtttaattactgtcggtaaagacggcagtgtaaatcttattggtggcaaagtgaaaaacgagggtgtgattagcgtaaatggtggcagcatttctttactcgcagggcaaaaaatcaccatcagcgatataataaacccaaccattacttacagcattgccgcgcctgaaaatgaagcggtcaatctgggcgatatttttgccaaaggcggtaacattaatgtccgtgctgccactattcgaaaccaaggtaaactttctgctgattctgtaagcaaagataaaagcggcaatattgttctttccgccaaagagggtgaagcggaaattggcggtgtaatttccgctcaaaatcagcaagctaaaggcggcaagctgatgattacaggcgataaagtcacattaaaaacaggtgcagttatcgacctttcaggtaaagaagggggagaaacttaccttggcggtgacgagcgcggcgaaggtaaaaacggcattcaattagcaaagaaaacctctttagaaaaaggctcaaccatcaatgtatcaggcaaagaaaaaggcggacgcgctattgtgtggggcgatattgcgttaattgacggcaatattaacgctcaaggtagtggtgatatcgctaaaaccggtggttttgtggagacatcggggcattatttatccattgacagcaatgcaattgttaaaacaaaagagtggttgctagaccctgatgatgtaacaattgaagccgaagacccccttcgcaataataccggtataaatgatgaattcccaacaggcaccggtgaagcaagcgaccctaaaaaaaatagcgaactcaaaacaacgctaaccaatacaactatttcaaattatctgaaaaacgcctggacaatgaatataacggcatcaagaaaacttaccgttaatagctcaatcaacatcggaagcaactcccacttaattctccatagtaaaggtcagcgtggcggaggcgttcagattgatggagatattacttctaaaggcggaaatttaaccatttattctggcggatgggttgatgttcataaaaatattacgcttgatcagggttttttaaatattaccgccgcttccgtagcttttgaaggtggaaataacaaagcacgcgacgcggcaaatgctaaaattgtcgcccagggcactgtaaccattacaggagagggaaaagatttcagggctaacaacgtatctttaaacggaacgggtaaaggtctgaatatcatttcatcagtgaataatttaacccacaatcttagtggcacaattaacatatctgggaatataacaattaaccaaactacgagaaagaacacctcgtattggcaaaccagccatgattcgcactggaacgtcagtgctcttaatctagagacaggcgcaaattttacctttattaaatacatttcaagcaatagcaaaggcttaacaacacagtatagaagctctgcaggggtgaattttaacggcgtaaatggcaacatgtcattcaatctcaaagaaggagcgaaagttaatttcaaattaaaaccaaacgagaacatgaacacaagcaaacctttaccaattcggtttttagccaatatcacagccactggtgggggctctgttttttttgatatatatgccaaccattctggcagaggggctgagttaaaaatgagtgaaattaatatctctaacggcgctaattttaccttaaattcccatgttcgcggcgatgacgcttttaaaatcaacaaagacttaaccataaatgcaaccaattcaaatttcagcctcagacagacgaaagatgatttttatgacgggtacgcacgcaatgccatcaattcaacctacaacatatccattctgggcggtaatgtcacccttggtggacaaaactcaagcagcagcattacggggaatattactatcgagaaagcagcaaatgttacgctagaagccaataacgcccctaatcagcaaaacataagggatagagttataaaacttggcagcttgctcgttaatgggagtttaagtttaactggcgaaaatgcagatattaaaggcaatctcactatttcagaaagcgccacttttaaaggaaagactagagataccctaaatatcaccggcaattttaccaataatggcactgccgaaattaatataacacaaggagtggtaaaacttggcaatgttaccaatgatggtgatttaaacattaccactcacgctaaacgcaaccaaagaagcatcatcggcggagatataatcaacaaaaaaggaagcttaaatattacagacagtaataatgatgctgaaatccaaattggcggcaatatctcgcaaaaagaaggcaacctcacgatttcttccgataaaattaatatcaccaaacagataacaatcaaaaagggtattgatggagaggactctagttcagatgcgacaagtaatgccaacctaactattaaaaccaaagaattgaaattgacagaagacctaagtatttcaggtttcaataaagcagagattacagccaaagatggtagagatttaactattggcaacagtaatgacggtaacagcggtgccgaagccaaaacagtaacttttaacaatgttaaagattcaaaaatctctgctgacggtcacaatgtgacactaaatagcaaagtgaaaacatctagcagcaatggcggacgtgaaagcaatagcgacaacgataccggcttaactattactgcaaaaaatgtagaagtaaacaaagatattacttctctcaaaacagtaaatatcaccgcgtcggaaaaggttaccaccacagcaggctcgaccattaacgcaacaaatggcaaagcaagtattacaaccaaaacaggtgatatcagcggtacgatttccggtaacacggtaagtgttagcgcgactggtgatttaaccactaaatccggctcaaaaattgaagcgaaatcgggtgaggctaatgtaacaagtgcaacaggtacaattggcggtacaatttccggtaatacggtaaatgttacggcaaacgctggcgatttaacagttgggaatggcgcagaaattaatgcgacagaaggagctgcaaccttaaccgcaacagggaataccttgactactgaagccggttctagcatcacttcaactaagggtcaggtagacctcttggctcagaatggtagcatcgcaggaagcattaatgctgctaatgtgacattaaatactacaggcaccttaaccaccgtggcaggctcggatattaaagcaaccagcggcaccttggttattaacgcaaaagatgctaagctaaatggtgatgcatcaggtgatagtacagaagtgaatgcagtcaacgcaagcggctctggtagtgtgactgcggcaacctcaagcagtgtgaatatcactggggatttaaacacagtaaatgggttaaatatcatttcgaaagatggtagaaacactgtgcgcttaagaggcaaggaaattgaggtgaaatatatccagccaggtgtagcaagtgtagaagaagtaattgaagcgaaacgcgtccttgaaaaagtaaaagatttatctgatgaagaaagagaaacattagctaaacttggtgtaagtgctgtacgttttgttgagccaaataatacaattacagtcaatacacaaaatgaatttacaaccagaccgtcaagtcaagtgataatttctgaaggtaaggcgtgtttctcaagtggtaatggcgcacgagtatgtaccaatgttgctgacgatggacagccgtagtcagtaattgacaaggtagatttcatcctgcaatgaagtcattttattttcgtattatttactgtgtgggttaaagttcagtacgggctttacccatcttgtaaaaaattacggagaatacaataaagtatttttaacaggttattattatg

SEQ ID NO: 4 - protein, 1477 aa, Haemophilus influenzae
MNKIYRLKFSKRLNALVAVSELARGCDHSTEKGSEKPARMKVRHLALKPLSAMLLSLGVTSIPQSVLASGLQGMDVVHGTATMQVDGNKTIIRNSVDAIINWKQFNIDQNEMVQFLQENNNSAVFNRVTSNQISQLKGILDSNGQVFLINPNGITIGKDAIINTNGFTASTLDISNENIKARNFTFEQTKDKALAEIVNHGLITVGKDGSVNLIGGKVKNEGVISVNGGSISLLAGQKITISDIINPTITYSIAAPENEAVNLGDIFAKGGNINVRAATIRNQGKLSADSVSKDKSGNIVLSAKEGEAEIGGVISAQNQQAKGGKLMITGDKVTLKTGAVIDLSGKEGGETYLGGDERGEGKNGIQLAKKTSLEKGSTINVSGKEKGGFAIVWGDIALIDGNINAQGSGDIAKTGGFVETSGHDLFIKDNAIVDAKEWLLDFDNVSINAEDPLFNNTGINDEFPTGTGEASDPKKNSELKTTLTNTTISNYLKNAWTMNITASRKLTVNSSINIGSNSHLILHSKGQRGGGVQIDGDITSKGGNLTIYSGGWVDVHKNITLDQGFLNITAASVAFEGGNNKARDAANAKIVAQGTVTITGEGKDFRANNVSLNGTGKGLNIISSVNNLTHNLSGTINISGNITINQTTRKNTSYWQTSHDSHWNVSALNLETGANFTFIKYISSNSKGLTTQYRSSAGVNFNGVNGNMSFNLKEGAKVNFKLKPNENMNTSKPLPIRFLANITATGGGSVFFDIYANHSGRGAELKMSEINISNGANFTLNSHVRGDDAFKINKDLTINATNSNFSLRQTKDDFYDGYARNAINSTYNISILGGNVTLGGQNSSSSITGNITIEKAANVTLEANNAPNQQNIRDRVIKLGSLLVNGSLSLTGENADIKGNLTISESATFKGKTRDTLNITGNFTNNGTAEINITQGVVKLGNVTNDGDLNITTHAKRNQRSIIGGDIINKKGSLNITDSNNDAEIQIGGNISQKEGNLTISSDKINITKQITIKKGIDGEDSSSDATSNANLTIKTKELKLTEDLSISGFNKAEITAKDGRDLTIGNSNDGNSGAEAKTVTFNNVKDSKISADGHNVTLNSKVKTSSSNGGRESNSDNDTGLTITAKNVEVNKDITSLKTVNITASEKVTTTAGSTINATNGKASITTKTGDISGTISGNTVSVSATVDLTTKSGSKIEAKSGEANVTSATGTIGGTISGNTVNVTANAGDLTVGNGAEINATEGAATLTATGNTLTTEAGSSITSTKGQVDLLAQNGSIAGSINAANVTLNTTGTLTTVAGSDIKATSGTLVINAKDAKLNGDASGDSTEVNAVNASGSGSVTAATSSSVNITGDLNTVNGLNIISKDGRNTVRLRGKEIEVKYIQPGVASVEEVIEAKRVLEKVKDLSDEERETLAKLGVSAVRFVEPNNTITVNTQNEFTTRPSSQVIISEGKACFSSGNGARVCTNVADDGQP

SEQ ID NO: 5 - DNA, 9171 bp, Haemophilus influenzae
acagcgttctcttaatactagtacaaacccacaataaaatatgacaaacaacaattacaacaccttttttgcagtctatatgcaaatattttaaaaaatagtataaatccgccatataaaatggtataatctttcatctttcatctttcatctttcatctttcatctttcatctttcatctttcatctttcatctttcatctttcatctttcatctttcatctttcatctttcatctttcacatgaaatgatgaaccgagggaagggagggaggggcaagaatgaagagggagctgaacgaacgcaaatgataaagtaatttaattgttcaactaaccttaggagaaaatatgaacaagatatatcgtctcaaattcagcaaacgcctgaatgctttggttgctgtgtctgaattggcacggggttgtgaccattccacagaaaaaggcagcgaaaaacctgctcgcatgaaagtgcgtcacttagcgttaaagccactttccgctatgttactatctttaggtgtaacatctattccacaatctgttttagcaagcggcttacaaggaatggatgtagtacacggcacagccactatgcaagtagatggtaataaaaccattatccgcaacagtgttgacgctatcattaattggaaacaatttaacatcgaccaaaatgaaatggtgcagtttttacaagaaaacaacaactccgccgtattcaaccgtgttacatctaaccaaatctcccaattaaaagggattttagattctaacggacaagtctttttaatcaacccaaatggtatcacaataggtaaagacgcaattattaacactaatggctttacggcttctacgctagacatttctaacgaaaacatcaaggcgcgtaatttcaccttcgagcaaaccaaagataaagcgctcgctgaaattgtgaatcacggtttaattactgtcggtaaagacggcagtgtaaatcttattggtggcaaagtgaaaaacgagggtgtgattagcgtaaatggtggcagcatttctttactcgcagggcaaaaaatcaccatcagcgatataataaacccaaccattacttacagcattgccgcgcctgaaaatgaagcggtcaatctgggcgatatttttgccaaaggcggtaacattaatgtccgtgctgccactattcgaaaccaagctttccgccaaagagggtgaagcggaaattggcggtgtaatttccgctcaaaatcagcaagctaaaggcggcaagctgatgattacaggcgataaagtcacattaaaaacaggtgcagttatcgacctttcaggtaaagaagggggagaaacttaccttggcggtgacgagcgcggcgaaggtaaaaacggcattcaattagcaaagaaaacctctttagaaaaaggctcaaccatcaatgtatcaggcaaagaaaaaggcggacgcgctattgtgtggggcgatattgcgttaattgacggcaatattaacgctcaaggtagtggtgatatcgctaaaaccggtggttttgtggagacgtcggggcatgatttattcatcaaagacaatgcaattgttgacgccaaagagtggttgttagacccggataatgtatctattaatgcagaaacagcaggacgcagcaatacttcagaagacgatgaatacacgggatccgggaatagtgccagcaccccaaaacgaaacaaagaaaagacaacattaacaaacacaactcttgagagtatactaaaaaaaggtacctttgttaacatcactgctaatcaacgcatctatgtcaatagctccattaatttatccaatggcagcttaactctttggagtgagggtcggagcggtggcggcgttgagattaacaacgatattaccaccggtgatgataccagaggtgcaaacttaacaatttactcaggcggctgggttgatgttcataaaaatatctcactcggggcgcaaggtaacataaacattacagctaaacaagatatcgcctttgagaaaggaagcaaccaagtcattacaggtcaagggactattacctcaggcaatcaaaaaggttttagatttaataatgtctctctaaacggcactggcagcggactgcaattcaccactaaaagaaccaataaatacgctatcacaaataaatttgaagggactttaaatatttcagggaaagtgaacatctcaatggttttacctaaaaatgaaagtggatatgataaattcaaaggacgcacttactggaatttaacctcgaaagtggatatgataaattcaaaggacgccctcactattgactccagaggaagcgatagtgcaggcacacttacccagccttataatttaaacggtatatcattcaacaaagacactacctttaatgttgaacgaaatgcaagagtcaactttgacatcaaggcaccaatagggataaataagtattctagtttgaattacgcatcatttaatggaaacatttcagtttcgggaggggggagtgttgatttcacacttctcgcctcatcctctaacgtccaaacccccggtgtagttataaattctaaatactttaatgtttcaacagggtcaagtttaagatttaaaacttcaggctcaacaaaaactggcttctcaatagagaaagatttaactttaaatgccaccggaggcaacataacacttttgcaagttgaaggcaccgatggaatgattggtaaaggcattgtagccaaaaaaaacataacctttgaaggaggtaagatgaggtttggctccaggaaagccgtaacagaaatcgaaggcaatgttactatcaataacaacgctaacgtcactcttatcggttcggattttgacaaccatcaaaaacctttaactattaaaaaagatgtcatcattaatagcggcaaccttaccgctggaggcaatattgtcaatatagccggaaatcttaccgttgaaagtaacgctaatttcaaagctatcacaaatttcacttttaatgtaggcggcttgtttgacaacaaaggcaattcaaatatttccattgccaaaggaggggctcgctttaaagacattgataattccaagaatttaagcatcaccaccaactccagctccacttaccgcactattataagcggcaatataaccaataaaaacggtgatttaaatattacgaacgaaggtagtgatactgaaatgcaaattggcggcgatgtctcgcaaaaagaaggtaatctcacgatttcttctgacaaaatcaatattaccaaacagataacaatcaaggcaggtgttgatggggagaattccgattcagacgcgacaaacaatgccaatctaaccattaaaaccaaagaattgaaattaacgcaagacctaaatatttcaggtttcaataaagcagagattacagctaaagatggtagtgatttaactattggtaacaccaatagtgctgatggtactaatgccaaaaaagtaacctttaaccaggttaaagattcaaaaatctctgctgacggtcacaaggtgacactacacagcaaagtggaaacatccggtagtaataacaacactgaagatagcagtgacaataatgccggcttaactatcgatgcaaaaaatgtaacagtaaacaacaatattacttctcacaaagcagtgagcatctctgcgacaagtggagaaattaccactaaaacaggtacaaccattaacgcaaccactggtaacgtggagataaccgctcaaacaggtagtatcctaggtggaattgagtccagctctggctctgtaacacttactgcaaccgagggcgctcttgctgtaagcaatatttcgggcaacaccgttactgttactgcaaatagcggtgcattaaccactttggcaggctctacaattaaaggaaccgagagtgtaaccacttcaagtcaatcaggcgatatcggcggtacgatttctggtggcacagtagaggttaaagcaaccgaaagtttaaccactcaatccaattcaaaaattaaagcaacaacaggcgaggctaacgtaacaagtgcaacaggtacaattggtggtacgatttccggtaatacggtaaatgttacggcaaacgctggcgatttaacagttgggaatggcgcagaaattaatgcgacagaaggagctgcaaccttaactacatcatcgggcaaattaactaccgaagctagttcacacattacttcagccaagggtcaggtaaatctttcagctcaggatggtagcgttgcaggaagtattaatgccgccaatgtgacactaaatactacaggcactttaactaccgtgaagggttcaaacattaatgcaaccagcggtaccttggttattaacgcaaaagacgctgagctaaatggcgcagcattgggtaaccacacagtggtaaatgcaaccaacgcaaatggctccggcagcgtaatcgcgacaacctcaagcagagtgaacatcactggggatttaatcacaataaatggattaaatatcatttcaaaaaacggtataaacaccgtactgttaaaaggcgttaaaattgatgtgaaatacattcaaccgggtatagcaagcgtagatgaagtaattgaagcgaaacgcatccttgagaaggtaaaagatttatctgatgaagaaagagaagcgttagctaaacttggcgtaagtgctgtacgttttattgagccaaataatacaattacagtcgatacacaaaatgaatttgcaaccagaccattaagtcgaatagtgatttctgaaggcagggcgtgtttctcaaacagtgatggcgcgacggtgtgcgttaatatcgctgataacgggcggtagcggtcagtaattgacaaggtagatttcatcctgcaatgaagtcattttattttcgtattatttactgtgtgggttaaagttcagtacgggctttacccatcttgtaaaaaattacggagaatacaataaagtatttttaacaggttattattatgaaaaatataaaaagcagattaaaactcagtgcaatatcagtattgcttggcctggcttcttcatcattgtatgcagaagaagcgtttttagtaaaaggctttcagttatctggtgcacttgaaactttaagtgaagacgcccaactgtctgtagcaaaatctttatctaaataccaaggctcgcaaactttaacaaacctaaaaacagcacagcttgaattacaggctgtgctagataagattgagccaaataagtttgatgtgatattgccacaacaaaccattacggatggcaatattatgtttgagctagtctcgaaatcagccgcagaaagccaagttttttataaggcgagccagggttatagtgaagaaaatatcgctcgtagcctgccatctttgaaacaaggaaaagtgtatgaagatggtcgtcagtggttcgatttgcgtgaattcaatatggcaaaagaaaatccacttaaagtcactcgcgtgcattacgagttaaaccctaaaaacaaaacctctgatttggtagttgcaggtttttcgccttttggcaaaacgcgtagctttgtttcctatgataatttcggcgcaagggagtttaactatcaacgtgtaagtctaggttttgtaaatgccaatttgaccggacatgatgatgtattaaatctaaacgcattgaccaatgtaaaagcaccatcaaaatcttatgcggtaggcataggatatacttatccgttttatgataaacaccaatccttaagtctttataccagcatgagttatgctgattctaatgatatcgacggcttaccaagtgcgattaatcgtaaattatcaaaaggtcaatctatctctgcgaatctgaaatggagttattatctcccgacatttaaccttggaatggaagaccagtttaaaattaatttaggctacaactaccgccatattaatcaaacatccgagttaaacaccctgggtgcaacgaagaaaaaatttgcagtatcaggcgtaagtgcaggcattgatggacatatccaatttacccctaaaacaatctttaatattgatttaactcatcattattacgcgagtaaattaccaggctcttttggaatggagcgcattggcgaaacatttaatcgcagctatcacattagcacagccagtttagggttgagtcaagagtttgctcaaggttggcattttagcagtcaattatcgggtcagtttactctacaagatataagtagcatagatttattctctgtaacaggtacttatggcgtcagaggctttaaatacggcggtgcaagtggtgagcgcggtcttgtatggcgtaatgaattaagtatgccaaaatacacccgctttcaaatcagcccttatgcgttttatgatgcaggtcagttccgttataatagcgaaaatgctaaaacttacggcgaagatatgcacacggtatcctctgcgggtttaggcattaaaacctctcctacacaaaacttaagcttagatgcttttgttgctcgtcgctttgcaaatgccaatagtgacaatttgaatggcaacaaaaaacgcacaagctcacctacaaccttctggggtagattaacattcagtttctaaccctgaaatttaatcaactggtaagcgttccgcctaccagtttataactatatgctttacccgccaatttacagtctatacgcaaccctgttttcatccttatatatcaaacaaactaagcaaaccaagcaaaccaagcaaaccaagcaaaccaagcaaaccaagcaaaccaagcaaaccaagcaaaccaagcaaaccaagcaaaccaagcaaaccaagcaaaccaagcaaaccaagcaatgctaaaaaacaatttatatgataaactaaaacatactccataccatggcaatacaagggatttaataatatgacaaaagaaaatttacaaagtgttccacaaaatacgaccgcttcacttgtagaatcaaacaacgaccaaacttccctgcaaatacttaaacaaccacccaaacccaacctattacgcctggaacaacatgtcgccaaaaaagattatgagcttgcttgccgcgaattaatggcgattttggaaaaaatggacgctaattttggaggcgttcacgatattgaatttgacgcacctgctcagctggcatatctacccgaaaaactactaattcattttgccactcgtctcgctaatgcaattacaacactcttttccgaccccgaattggcaatttccgaagaaggggcattaaagatgattagcctgcaacgctggttgacgctgatttttgcctcttccccctacgttaacgcagaccatattctcaataaatataatatcaacccagattccgaaggtggctttcatttagcaacagacaactcttctattgctaaattctgtattttttacttacccgaatccaatgtcaatatgagtttagatgcgttatgggcagggaatcaacaactttgtgcttcattgtgttttgcgttgcagtcttcacgttttattggtactgcatctgcgtttcataaaagagcggtggttttacagtggtttcctaaaaaactcgccgaaattgctaatttagatgaattgcctgcaaatatccttcatgatgtatatatgcactgcagttatgatttagcaaaaaacaagcacgatgttaagcgtccattaaacgaacttgtccgcaagcatatcctcacgcaaggatggcaagaccgctacctttacaccttaggtaaaaaggacggcaaacctgtgatgatggtactgcttgaacattttaattcgggacattcgatttatcgcacgcattcaacttcaatgattgctgctcgagaaaaattctatttagtcggcttaggccatgagggcgttgataacataggtcgagaagtgtttgacgagttctttgaaatcagtagcaataatataatggagagactgttttttatccgtaaacagtgcgaaactttccaacccgcagtgttctatatgccaagcattggcatggatattaccacgatttttgtgagcaacactcggcttgcccctattcaagctgtagccttgggtcatcctgccactacgcattctgaatttattgattatgtcatcgtagaagatgattatgtgggcagtgaagattgttttagcgaaacccttttacgcttacccaaagatgccctaccttatgtaccatctgcactcgccccacaaaaagtggattatgtactcagggaaaaccctgaagtagtcaatatcggtattgccgctaccacaatgaaattaaaccctgaatttttgctaacattgcaagaaatcagagataaagctaaagtcaaaatacattttcatttcgcacttggacaatcaacaggcttgacacacccttatgtcaaatggtttatcgaaagctatttaggtgacgatgccactgcacatccccacgcaccttatcacgattatctggcaatattgcgtgattgcgatatgctactaaatccgtttcctttcggtaatactaacggcataattgatatggttacattaggtttagttggtgtatgcaaaacgggggatgaagtacatgaacatattgatgaaggtctgtttaaacgcttaggactaccagaatggctgatagccgacacacgagaaacatatattgaatgtgctttgcgtctagcagaaaaccatcaagaacgccttgaactccgtcgttacatcatagaaaacaacggcttacaaaagctttttacaggcgaccctcgtccattgggcaaaatactgcttaagaaaacaaatgaatggaagcggaagcacttgagtaaaaaataacggttttttaaagtaaaagtgcggttaattttcaaagcgttttaaaaacctctcaaaaatcaaccgcacttttatctttataacgctcccgcgcgctgacagtttatctctttcttaaaatacccataaaattgtggcaatagttgggtaatcaaattcaattgttgatacggcaaactaaagacggcgcgttcttcggcagtcatc

SEQ ID NO: 6 - DNA, 9323 bp, Haemophilus influenzae
cgccacttcaattttggattgttgaaattcaactaaccaaaaagtgcggttaaaatctgtggagaaaataggttgtagtgaagaacgaggtaattgttcaaaaggataaagctctcttaattgggcattggttggcgtttctttttcggttaatagtaaattatattctggacgactatgcaatccaccaacaactttaccgttggttttaagcgttaatgtaagttcttgctcttcttggcgaatacgtaatcccattttttgtttagcaagaaaatgatcgggataatcataataggtgttgcccaaaaataaattttgatgttctaaaatcataaattttgcaagatattgtggcaattcaatacctatttgtggcgaaatcgccaattttaattcaatttcttgtagcataatatttcccactcaaatcaactggttaaatatacaagataataaaaataaatcaagatttttgtgatgacaaacaacaattacaacaccttttttgcagtctatatgcaaatattttaaaaaaatagtataaatccgccatataaaatggtataatctttcatctttcatctttcatctttcatctttcatctttcatctttcatctttcatctttcatctttcatctttcatctttcatctttcatctttcatctttcatctttcacatgaaatgatgaaccgagggaagggagggaggggcaagaatgaagagggagctgaacgaacgcaaatgataaagtaatttaattgttcaactaaccttaggagaaaatatgaacaagatatatcgtctcaaattcagcaaacgcctgaatgctttggttgctgtgtctgaattggcacggggttgtgaccattccacagaaaaaggcagcgaaaaacctgctcgcatgaaagtgcgtcacttagcgttaaagccactttccgctatgttactatctttaggtgtaacatctattccacaatctgttttagcaagcggcaatttaacatcgaccaaaatgaaatggtgcagtttttacaagaaaacaagtaataaaaccattatccgcaacagtgttgacgctatcattaattggaaacaatttaacatcgaccaaaatgaaatggtgcagtttttacaagaaaacaacaactccgccgtattcaaccgtgttacatctaaccaaatctcccaattaaaagggattttagattctaacggacaagtctttttaatcaacccaaatggtatcacaataggtaaagacgcaattattaacactaatggctttacggcttctacgctagacatttctaacgaaaacatcaaggcgcgtaatttcaccttcgagcaaaccaaagataaagcgctcgctgaaattgtgaatcacggtttaattactgtcggtaaagacggcagtgtaaatcttattggtggcaaagtgaaaaacgagggtgtgattagcgtaaatggtggcagcatttctttactcgcagggcaaaaaatcaccatcagcgatataataaacccaaccattacttacagcattgccgcgcctgaaaatgaagcggtcaatctgggcgatatttttgccaaaggcggtaacattaatgtccgtgctgccactattcgaaaccaaggtaaactttctgctgattctgtaagcaaagataaaagcggcaatattgttctttccgccaaagagggtgaagcggaaattggcggtgtaatttccgctcaaaatcagcaagctaaaggcggcaagctgatgataaagtccgataaagtcacattaaaaacaggtgcagttatcgacctttcaggtaaagaagggggagaaacttaccttggcggtgacgagcgcggcgaaggtaaaaacggcattcaattagcaaagaaaacctctttagaaaaaggctcaaccatcaatgtatcaggcaaagaaaaaggcggacgcgctattgtgtggggcgatattgcgttaattgacggcaatattaacgctcaaggtagtggtgatatcgctaaaaccggtggttttgtggagacatcggggcattatttatccattgacagcaatgcaattgttaaaacaaaagagtggttgctagaccctgatgatgtaacaattgaagccgaagacccccttcgcaataataccggtataaatgatgaattcccaacaggcaccggtgaagcaagcgaccctaaaaaaaatagcgaactcaaaacaacgctaaccaatacaactatttcaaattatctgaaaaacgcctggacaatgaatataacggcatcaagaaaacttaccgttaatagctcaatcaacatcggaagcaactcccacttaattctccatagtaaaggtcagcgtggcggaggcgttcagattgatggagatattacttctaaaggcggaaatttaaccatttattctggcggatgggttgatgttcataaaaatattacgcttgatcagggttttttaaatattaccgccgcttccgtagcttttgaaggtggaaataacaaagcacgcgacgcggcaaatgctaaaattgtcgcccagggcactgtaaccattacaggagagggaaaagatttcagggctaacaacgtatctttaaacggaacgggtaaaggtctgaatatcatttcatcagtgaataatttaacccacaatcttagtggcacaattaacatatctgggaatataacaattaaccaaactacgagaaagaacacctcgtattggcaaaccagccatgattcgcactggaacgtcagtgctcttaatctagagacaggcgcaaattttacctttattaaatacatttcaagcaatagcaaaggcttaacaacacagtatagaagctctgcaggggtgaattttaacggcgtaaatggcaacatgtcattcaatctcaaagaaggagcgaaagttaatttcaaattaaaaccaaacgagaacatgaacacaagcaaacctttaccaattcggtttttagccaatatcacagccactggtgggggctctgttttttttgatatatatgccaaccattctggcagaggggctgagttaaaaatgagtgaaattaatatctctaacggcgctaattttaccttaaattcccatgttcgcggcgatgacgcttttaaaatcaacaaagacttaaccataaatgcaaccaattcaaatttcagcctcagacagacgaaagatgatttttatgacgggtacgcacgcaatgccatcaattcaacctacaacatatccattctgggcggtaatgtcacccttggtggacaaaactcaagcagcagcattacggggaatattactatcgagaaagcagcaaatgttacgctagaagccaataacgcccctaatcagcaaaacataagggatagagttataaaacttggcagcttgctcgttaatgggagtttaagtttaactggcgaaaatgcagatattaaaggcaatctcactatttcagaaagcgccacttttaaaggaaagactagagataccctaaatatcaccggcaattttaccaataatggcactgccgaaattaatataacacaaggagtggtaaaacttggcaatgttaccaatgatggtgatttaaacattaccactcacgctaaacgcaaccaaagaagcatcatcggcggagatataatcaacaaaaaaggaagcttaaatattacagacagtaataatgatgctgaaatccaaattggcggcaatatctcgcaaaaagaaggcaacctcacgatttcttccgataaaattaatatcaccaaacagataacaatcaaaaagggtattgatggagaggactctagttcagatgcgacaagtaatgccaacctaactattaaaaccaaagaattgaaattgacagaagacctaagtatttcaggtttcaataaagcagagattacagccaaagatggtagagatttaactattggcaacagtaatgacggtaacagcggtgccgaagccaaaacagtaacttttaacaatgttaaagattcaaaaatctctgctgacggtcacaatgtgacactaaatagcaaagtgaaaacatctagcagcaatggcggacgtgaaagcaatagcgacaacgataccggcttaactattactgcaaaaaatgtagaagtaaacaaagatattacttctctcaaaacagtaaatatcaccgcgtcggaaaaggttaccaccacagcaggctcgaccattaacgcaacaaatggcaaagcaagtattacaaccaaaacaggtgatatcagcggtacgatttccggtaacacggtaagtgttagcgcgactggtgatttaaccactaaatccggctcaaaaattgaagcgaaatcgggtgaggctaatgtaacaagtgcaacaggtacaattggcggtacaatttccggtaatacggtaaatgttacggcaaacgctggcgatttaacagttgggaatggcgcagaaattaatgcgacagaaggagctgcaaccttaaccgcaacagggaataccttgactactgaagccggttctagcatcacttcaactaagggtcaggtagacctcttggctcagaatggtagcatcgcaggaagcattaatgctgctaatgtgacattaaatactacaggcaccttaaccaccgtggcaggctcggatattaaagcaaccagcggcaccttggttattaacgcaaaagatgctaagctaaatggtgatgcatcaggtgatagtacagaagtgaatgcagtcaacgactggggatttggtagtgtgactgcggcaacctcaagcagtgtgaatatcactggggatttaaacacagtaaatgggttaaatatcatttcgaaagatggtagaaacactgtgcgcttaagaggcaaggaaattgaggtgaaatatatccagccaggtgtagcaagtgtagaagaagtaattgaagcgaaacgcgtccttgaaaaagtaaaagatttatctgatgaagaaagagaaacattagctaaacttggtgtaagtgctgtacgttttgttgagccaaataatacaattacagtcaatacacaaaatgaatttacaaccagaccgtcaagtcaagtgataatttctgaaggtaaggcgtgtttctcaagtggtaatggcgcacgagtatgtaccaatgttgctgacgatggacagccgtagtcagtaattgacaaggtagatttcatcctgcaatgaagtcattttattttcgtattatttactgtgtgggttaaagttcagtacgggctttacccatcttgtaaaaaattacggagaatacaataaagtatttttaacaggttattattatgaaaaatataaaaagcagattaaaactcagtgcaatatcagtattgcttggcctggcttcttcatcattgtatgcagaagaagcgtttttagtaaaaggctttcagttatctggtgcacttgaaactttaagtgaagacgcccaactgtctgtagcaaaatctttatctaaataccaaggctcgcaaactttaacaaacctaaaaacagcacagcttgaattacaggctgtgctagataagattgagccaaataaatttgatgtgatattgccgcaacaaaccattacggatggcaatatcatgtttgagctagtctcgaaatcagccgcagaaagccaagttttttataaggcgagccagggttatagtgaagaaaatatcgctcgtagcctgccatctttgaaacaaggaaaagtgtatgaagatggtcgtcagtggttcgatttgcgtgaatttaatatggcaaaagaaaacccgcttaaggttacccgtgtacattacgaactaaaccctaaaaacaaaacctctaatttgataattgcgggcttctcgccttttggtaaaacgcgtagctttatttcttatgataatttcggcgcgagagagtttaactaccaacgtgtaagcttgggttttgttaatgccaatttaactggtcatgatgatgtgttaattataccagtatgagttatgctgattctaatgatatcgacggcttaccaagtgcgattaatcgtaaattatcaaaaggtcaatctatctctgcgaatctgaaatggagttattatctcccaacatttaaccttggcatggaagaccaatttaaaattaatttaggctacaactaccgccatattaatcaaacctccgcgttaaatcgcttgggtgaaacgaagaaaaaatttgcagtatcaggcgtaagtgcaggcattgatggacatatccaatttacccctaaaacaatctttaatattgatttaactcatcattattacgcgagtaaattaccaggctcttttggaatggagcgcattggcgaaacatttaatcgcagctatcacattagcacagccagtttagggttgagtcaagagtttgctcaaggttggcattttagcagtcaattatcaggtcaatttactctacaagatattagcagtatagatttattctctgtaacaggtacttatggcgtcagaggctttaaatacggcggtgcaagtggtgagcgcggtcttgtatggcgtaatgaattaagtatgccaaaatacacccgcttccaaatcagcccttatgcgttttatgatgcaggtcagttccgttataatagcgaaaatgctaaaacttacggcgaagatatgcacacggtatcctctgcgggtttaggcattaaaacctctcctacacaaaacttaagcctagatgcttttgttgctcgtcgctttgcaaatgccaatagtgacaatttgaatggcaacaaaaaacgcacaagctcacctacaaccttctgggggagattaacattcagtttctaaccctgaaatttaatcaactggtaagcgttccgcctaccagtttataactatatgctttacccgccaatttacagtctataggcaaccctgtttttacccttatatatcaaataaacaagctaagctgagctaagcaaaccaagcaaactcaagcaagccaagtaatactaaaaaaacaatttatatgataaactaaagtatactccatgccatggcgatacaagggatttaataatatgacaaaagaaaatttgcaaaacgctcctcaagatgcgaccgctttacttgcggaattaagcaacaatcaaactcccctgcgaatatttaaacaaccacgcaagcccagcctattacgcttggaacaacatatcgcaaaaaaagattatgagtttgcttgtcgtgaattaatggtgattctggaaaaaatggacgctaattttggaggcgttcacgatattgaatttgacgcacccgctcagctggcatatctacccgaaaaattactaatttattttgccactcgtctcgctaatgcaattacaacactcttttccgaccccgaattggcaatttctgaagaaggggcgttaaagatgattagcctgcaacgctggttgacgctgatttttgcctcttccccctacgttaacgcagaccatattctcaataaatataatatcaacccagattccgaaggtggctttcatttagcaacagacaactcttctattgctaaattctgtattttttacttacccgaatccaatgtcaatatgagtttagatgcgttatgggcagggaatcaacaactttgtgcttcattgtgttttgcgttgcagtcttcacgttttattggtaccgcatctgcgtttcataaaagagcggtggttttacagtggtttcctaaaaaactcgccgaaattgctaatttagatgaattgcctgcaaatatccttcatgatgtatatatgcactgcagttatgatttagcaaaaaacaagcacgatgttaagcgtccattaaacgaacttgtccgcaagcatatcctcacgcaaggatggcaagaccgctacctttacaccttaggtaaaaaggacggcaaacctgtgatgatggtactgcttgaacattttaattcgggacattcgatttatcgtacacattcaacttcaatgattgctgctcgagaaaaattctatttagtcggcttaggccatgagggcgttgataaaataggtcgagaagtgtttgacgagttctttgaaatcagtagcaataatataatggagagactgttttttatccgtaaacagtgcgaaactttccaacccgcagtgttctatatgccaagcattggcatggatattaccacgatttttgtgagcaacactcggcttgcccctattcaagctgtagccctgggtcatcctgccactacgcattctgaatttattgattatgtcatcgtagaagatgattatgtgggcagtgaagattgtttcagcgaaacccttttacgcttacccaaagatgccctaccttatgtaccttctgcactcgccccacaaaaagtggattatgtactcagggaaaaccctgaagtagtcaatatcggtattgccgctaccacaatgaaattaaaccctgaatttttgctaacattgcaagaaatcagagataaagctaaagtcaaaatacattttcatttcgcacttggacaatcaacaggcttgacacacccttatgtcaaatggtttatcgaaagctatttaggtgacgatgccactgcacatccccacgcaccttatcacgattatctggcaatattgcgtgattgcgatatgctactaaatccgtttcctttcggtaatactaacggcataattgatatggttacattaggtttagttggtgtatgcaaaacgggggatgaagtacatgaacatattgatgaaggtctgtttaaacgcttaggactaccagaatggctgatagccgacacacgagaaacatatattgaatgtgctttgcgtctagcagaaaaccatcaagaacgccttgaactccgtcgttacatcatagaaaacaacggcttacaaaagctttttacaggcgaccctcgtccattgggcaaaatactgcttaagaaaacaaatgaatggaagcggaagcacttgagtaaaaaataacggttttttaaagtaaaagtgcggttaattttcaaagcgttttaaaaacctctcaaaaatcaaccgcacttttatctttataacgatcccgcacgctgacagtttatcagcctcccgccataaaactccgcctttcatggcggagattttagccaaaactggcagaaattaaaggctaaaatcaccaaattgcaccacaaaatcaccaatacccacaaaaaa

SEQ ID NO: 7 - DNA, 4794 bp, Haemophilus influenzae
atgaacaagatatatcgtctcaaattcagcaaacgcctgaatgctttggttgctgtgtctgaattgacacggggttgtgaccattccacagaaaaaggcagtgaaaaacctgttcgtacgaaagtacgccacttggcgttaaagccactttccgctatattgctatctttgggcatggcatccattccgcaatctgttttagcgagcggtttacagggaatgagcgtcgtacacggtacagcaaccatgcaagtagacggcaataaaaccactatccgtaatagcgtcaatgctatcatcaattggaaacaatttaacattgaccaaaatgaaatggtgcagtttttacaagaaagcagcaactctgccgttttcaaccgtgttacatctgaccaaatctcccaattaaaagggattttagattctaacggacaagtctttttaatcaacccaaatggtatcacaataggtaaagacgcaattattaacactaatggctttactgcttctacgctagacatttctaacgaaaacatcaaggcgcgtaatttcacccttgagcaaaccaaggataaagcactcgctgaaatcgtgaatcacggtttaattaccgttggtaaagacggtagcgtaaaccttattggtggcaaagtgaaaaacgagggcgtgattagcgtaaatggcggtagtatttctttacttgcagggcaaaaaatcaccatcagcgatataataaatccaaccatcacttacagcattgctgcacctgaaaacgaagcgatcaatctgggcgatatttttgccaaaggtggtaacattaatgtccgcgctgccactattcgcaataaaggtaaactttctgccgactctgtaagcaaagataaaagtggtaacattgttctctctgccaaagaaggtgaagcggaaattggcggtgtaatttccgctcaaaatcagcaagccaaaggtggtaagttgatgattacaggcgataaagttacattgaaaacgggtgcagttatcgacctttcgggtaaagaagggggagaaacttatcttggcggtgacgagcgtggcgaaggtaaaaacggcattcaattagcaaagaaaaccactttagaaaaaggctcaacaattaatgtgtcaggtaaagaaaaaggtgggcgcgctattgtatggggcgatattgcgttaattgacggcaatattaatgcccaaggtaaagatatcgctaaaactggtggttttgtggagacgtcggggcattacttatccattgatgataacgcaattgttaaaacaaaagaatggctactagacccagagaatgtgactattgaagctccttccgcttctcgcgtcgagctgggtgccgataggaattcccactcggcagaggtgataaaagtgaccctaaaaaaaaataacacctccttgacaacactaaccaatacaaccatttcaaatcttctgaaaagtgcccacgtggtgaacataacggcaaggagaaaacttaccgttaatagctctatcagtatagaaagaggctcccacttaattctccacagtgaaggtcagggcggtcaaggtgttcagattgataaagatattacttctgaaggcggaaatttaaccatttattctggcggatgggttgatgttcataaaaatattacgcttggtagcggctttttaaacatcacaactaaagaaggagatatcgccttcgaagacaagtctggacggaacaacctaaccattacagcccaagggaccatcacctcaggtaatagtaacggctttagatttaacaacgtctctctaaacagccttggcggaaagctgagctttactgacagcagagaggacagaggtagaagaactaagggtaatatctcaaacaaatttgacggaacgttaaacatttccggaactgtagatatctcaatgaaagcacccaaagtcagctggttttacagagacaaaggacgcacctactggaacgtaaccactttaaatgttacctcgggtagtaaatttaacctctccattgacagcacaggaagtggctcaacaggtccaagcatacgcaatgcagaattaaatggcataacatttaataaagccacttttaatatcgcacaaggctcaacagctaactttagcatcaaggcatcaataatgccctttaagagtaacgctaactacgcattatttaatgaagatatttcagtctcaggggggggtagccttaatttcaaacttaacgcctcatctagcaacatacaaacccctggcgtaattataaaatctcaaaactttaatgtctcaggagggtcaactttaaatctcaaggctgaaggttcaacagaaaccgctttttcaatagaaaatgatttaaacttaaacgccaccggtggcaatataacaatcagacaagtcgagggtaccgattcacgcgtcaacaaaggtgtcgcagccaaaaaaaacataacttttaaagggggtaatatcaccttcggctctcaaaaagccacaacagaaatcaaaggcaatgttaccatcaataaaaacactaacgctactctttgtggtgcgaattttgccgaaaacaaatcgcctttaaatatagcaggaaatgttattaataatggcaaccttaccactgccggctccattatcaatatagccggaaatcttactgtttcaaaaggcgctaaccttcaagctataacaaattacacttttaatgtagccggctcatttgacaacaatggcgcttcaaacatttccattgccagaggaggggctaaatttaaagatatcaataacaccagtagcttaaatattaccaccaactctgataccacttaccgcaccattataaaaggcaatatatccaacaaatcaggtgatttgaatattattgataaaaaaagcgacgctgaaatccaaattggcggcaatatctcacaaaaagaaggcaatctcacaatttcttctgataaagtaaatattaccaatcagataacaatcaaagcaggcgttgaaggggggcgttctgattcaagtgaggcagaaaatgctaacctaactattcaaaccaaagagttaaaattggcaggagacctaaatatttcaggctttaataaagcagaaattacagctaaaaatggcagtgatttaactattggcaatgctagcggtggtaatgctgatgctaaaaaagtgacttttgacaaggttaaagattcaaaaatctcgactgacggtcacaatgtaacactaaatagcgaagtgaaaacgtctaatggtagtagcaatgctggtaatgataacagcaccggtttaaccatttccgcaaaagatgtaacggtaaacaataacgttacctcccacaagacaataaatatctctgccgcagcaggaaatgtaacaaccaaagaaggcacaactatcaatgcaaccacaggcagcgtggaagtaactgctcaaaatggtacaattaaaggcaacattacctcgcaaaatgtaacagtgacagcaacagaaaatcttgttaccacagagaatgctgtcattaatgcaaccagcggcacagtaaacattagtacaaaaacaggggatattaaaggtggaattgaatcaacttccggtaatgtaaatattacagcgagcggcaatacacttaaggtaagtaatatcactggtcaagatgtaacagtaacagcggatgcaggagccttgacaactacagcaggctcaaccattagtgcgacaacaggcaatgcaaatattacaaccaaaacaggtgatatcaacggtaaagttgaatccagctccggctctgtaacacttgttgcaactggagcaactcttgctgtaggtaatatttcaggtaacactgttactattactgcggatagcggtaaattaacctccacagtaggttctacaattaatgggactaatagtgtaaccacctcaagccaatcaggcgatattgaaggtacaatttctggtaatacagtaaatgttacagcaagcactggtgatttaactattggaaatagtgcaaaagttgaagcgaaaaatggagctgcaaccttaactgctgaatcaggcaaattaaccacccaaacaggctctagcattacctcaagcaatggtcagacaactcttacagccaaggatagcagtatcgcaggaaacattaatgctgctaatgtgacgttaaataccacaggcactttaactactacaggggattcaaagattaacgcaaccagtggtaccttaacaatcaatgcaaaagatgccaaattagatggtgctgcatcaggtgaccgcacagtagtaaatgcaactaacgcaagtggctctggtaacgtgactgcgaaaacctcaagcagcgtgaatatcaccggggatttaaacacaataaatgggttaaatatcatttcggaaaatggtagaaacactgtgcgcttaagaggcaaggaaattgatgtgaaatatatccaaccaggtgtagcaagcgtagaagaggtaattgaagcgaaacgcgtccttgagaaggtaaaagatttatctgatgaagaaagagaaacactagccaaacttggtgtaagtgctgtacgtttcgttgagccaaataatgccattacggttaatacacaaaacgagtttacaaccaaaccatcaagtcaagtgacaatttctgaaggtaaggcgtgtttctcaagtggtaatggcgcacgagtatgtaccaatgttgctgacgatggacagcag

SEQ ID NO: 8 - DNA, 4803 bp, Haemophilus influenzae
atgaacaagatatatcgtctcaaattcagcaaacgcctgaatgctttggttgctgtgtctgaattgacacggggttgtgaccattccacagaaaaaggcagtgaaaaacctgttcgtacgaaagtacgccacttggcgttaaagccactttccgctatattgctatctttgggcatggcatccattccgcaatctgttttagcgagcggtttacagggaatgagcgtcgtacacggtacagcaaccatgcaagtagacggcaataaaaccactatccgtaatagcgtcaatgctatcatcaattggaaacaatttaacattgaccaaaatgaaatggtgcagtttttacaagaaagcagcaactctgccgttttcaaccgtgttacatctgaccaaatctcccaattaaaagggattttagattctaacggacaagtctttttaatcaacccaaatggtatcacaataggtaaagacgcaattattaacactaatggctttactgcttctacgctagacatttctaacgaaaacatcaaggcgcgtaatttcacccttgagcaaaccaaggataaagcactcgctgaaatcgtgaatcacggtttaattaccgttggtaaagacggtagcgtaaaccttattggtggcaaagtgaaaaacgagggcgtgattagcgtaaatggcggtagtatttctttacttgcagggcaaaaaatcaccatcagcgatataataaatccaaccatcacttacagcattgctgcacctgaaaacgaagcgatcaatctgggcgatatttttgccaaaggtggtaacattaatgtccgcgctgccactattcgcaataaaggtaaactttctgccgactctgtaagcaaagataaaagtggtaacattgttctctctgccaaagaaggtgaagcggaaattggcggtgtaatttccgctcaaaatcagcaagccaaaggtggtaagttgatgattacaggtgataaagtcacattaaaaacaggtgcagttatcgacctttcaggtaaagaagggggagagacttatcttggcggtgatgagcgtggcgaaggtaaaaatggtattcaattagcgaagaaaacctctttagaaaaaggctcgacaattaatgtatcaggcaaagaaaaaggcgggcgcgctattgtatggggcgatattgcattaattaatggtaacattaatgctcaaggtagcgatattgctaaaactggcggctttgtggaaacatcaggacatgacttatccattggtgatgatgtgattgttgacgctaaagagtggttattagacccagatgatgtgtccattgaaactcttacatctggacgcaataataccggcgaaaaccaaggatatacaacaggagatgggactaaagagtcacctaaaggtaatagtatttctaaacctacattaacaaactcaactcttgagcaaatcctaagaagaggttcttatgttaatatcactgctaataatagaatttatgttaatagctccatcaacttatctaatggcagtttaacacttcacactaaacgagatggagttaaaattaacggtgatattacctcaaacgaaaatggtaatttaaccattaaagcaggctcttgggttgatgttcataaaaacatcacgcttggtacgggttttttgaatattgtcgctggggattctgtagcttttgagagagagggcgataaagcacgtaacgcaacagatgctcaaattaccgcacaagggacgataaccgtcaataaagatgataaacaatttagattcaataatgtatctattaacgggacgggcaagggtttaaagtttattgcaaatcaaaataatttcactcataaatttgatggcgaaattaacatatctggaatagtaacaattaaccaaaccacgaaaaaagatgttaaatactggaatgcatcaaaagactcttactggaatgtttcttctcttactttgaatacggtgcaaaaatttacctttataaaattcgttgatagcggctcaaattcccaagatttgaggtcatcacgtagaagttttgcaggcgtacattttaacggcatcggaggcaaaacaaacttcaacatcggagctaacgcaaaagccttatttaaattaaaaccaaacgccgctacagacccaaaaaaagaattacctattacttttaacgccaacattacagctaccggtaacagtgatagctctgtgatgtttgacatacacgccaatcttacctctagagctgccggcataaacatggattcaattaacattaccggcgggcttgacttttccataacatcccataatcgcaatagtaatgcttttgaaatcaaaaaagacttaactataaatgcaactggctcgaattttagtcttaagcaaacgaaagattctttttataatgaatacagcaaacacgccattaactcaagtcataatctaaccattcttggcggcaatgtcactctaggtggggaaaattcaagcagtagcattacgggcaatatcaatatcaccaataaagcaaatgttacattacaagctgacaccagcaacagcaacacaggcttgaagaaaagaactctaactcttggcaatatatctgttgaggggaatttaagcctaactggtgcaaatgcaaacattgtcggcaatctttctattgcagaagattccacatttaaaggagaagccagtgacaacctaaacatcaccggcacctttaccaacaacggtaccgccaacattaatataaaacaaggagtggtaaaactccaaggcgatattatcaataaaggtggtttaaatatcactactaacgcctcaggcactcaaaaaaccattattaacggaaatataactaacgaaaaaggcgacttaaacatcaagaatattaaagccgacgccgaaatccaaattggcggcaatatctcacaaaaagaaggcaatctcacaatttcttctgataaagtaaatattaccaatcagataacaatcaaagcaggcgttgaaggggggcgttctgattcaagtgaggcagaaaatgctaacctaactattcaaaccaaagagttaaaattggcaggagacctaaatatttcaggctttaataaagcagaaattacagctaaaaatggcagtgatttaactattggcaatgctagcggtggtaatgctgatgctaaaaaagtgacttttgacaaggttaaagattcaaaaatctcgactgacggtcacaatgtaacactaaatagcgaagtgaaaacgtctaatggtagtagcaatgctggtaatgataacagcaccggtttaaccatttccgcaaaagatgtaacggtaaacaataacgttacctcccacaagacaataaatatctctgccgcagcaggaaatgtaacaaccaaagaaggcacaactatcaatgcaaccacaggcagcgtggaagtaactgctcaaaatggtacaattaaaggcaacattacctcgcaaaatgtaacagtgacagcaacagaaaatcttgttaccacagagaatgctgtcattaatgcaaccagcggcacagtaaacattagtacaaaaacaggggatattaaaggtggaattgaatcaacttccggtaatgtaaatattacagcgagcggcaatacacttaaggtaagtaatatcactggtcaagatgtaacagtaacagcggatgcaggagccttgacaactacagcaggctcaaccattagtgcgacaacaggcaatgcaaatattacaaccaaaacaggtgatatcaacggtaaagttgaatccagctccggctctgtaacacttgttgcaactggagcaactcttgctgtaggtaatatttcaggtaacactgttactattactgcggatagcggtaaattaacctccacagtaggttctacaattaatgggactaatagtgtaaccacctcaagccaatcaggcgatattgaaggtacaatttctggtaatacagtaaatgttacagcaagcactggtgatttaactattggaaatagtgcaaaagttgaagcgaaaaatggagctgcaaccttaactgctgaatcaggcaaattaaccacccaaacaggctctagcattacctcaagcaatggtcagacaactcttacagccaaggatagcagtatcgcaggaaacattaatgctgctaatgtgacgttaaataccacaggcactttaactactacaggggattcaaagattaacgcaaccagtggtaccttaacaatcaatgcaaaagatgccaaattagatggtgctgcatcaggtgaccgcacagtagtaaatgcaactaacgcaagtggctctggtaacgtgactgcgaaaacctcaagcagcgtgaatatcaccggggatttaaacacaataaatgggttaaatatcatttcggaaaatggtagaaacactgtgcgcttaagaggcaaggaaattgatgtgaaatatatccaaccaggtgtagcaagcgtagaagaggtaattgaagcgaaacgcgtccttgagaaggtaaaagatttatctgatgaagaaagagaaacactagccaaacttggtgtaagtgctgtacgtttcgttgagccaaataatgccattacggttaatacacaaaacgagtttacaaccaaaccatcaagtcaagtgacaatttctgaaggtaaggcgtgtttctcaagtggtaatggcgcacgagtatgtaccaatgttgctgacgatggacagcag

SEQ ID NO: 9 - protein, 1599 aa, Haemophilus influenzae
MNKIYRLKFSKRLNALVAVSELTRGCDHSTEKGSEKPVRTKVRHLALKPLSAILLSLGMASIPQSVLASGLQGMSVVHGTATMQVDGNKTTIRNSVNAIINWKQFNIDQNEMEQFLQESSNSAVFNRVTSDQISQLKGILDSNGQVFLINPNGITIGKDAIINTNGFTASTLDISNENIKARNFTLEQTKDKALAEIVNHGLITVGKDGSVNLIGGKVKNEGVISVNGGSISLLAGQKITISDIINPTITYSIAAPENEAINLGDIFAKGGNINVRAATIRNKGKLSADSVSKDKSGNIVLSAKEGEAEIGGVISAQNQQAKGGKLMITGDKVTLKTGAVIDLSGKEGGETYLGGDERGEGKNGIQLAKKTTLEKGSTINVSGKEKGGRAIVWGDIALIDGNINAQGKDIAKTGGFVETSGHYLSIDDNAIVKTKEWLLDPENVTIEAPSASRVELGADRNSHSAEVIKVTLKKNNTSLTTLTNTTISNLLKSAHVVNITARRKLTVNSSISIERGSHLILHSEGQGGQGVQIDKDITSEGGNLTIYSGGWVDVHKNITLGSGFLNITTKEGDIAFEDKSGRNNLTITAQGTITSGNSNGFRFNNVSLNSLGGKLSFTDSREDRGRRTKGNISNKFDGTLNISGTVDISMKAPKVSWFYRDKGRTYWNVTTLNVTSGSKFNLSIDSTGSGSTGPSIRNAELNGITFNKATFNIAQGSTANFSIKASIMPFKSNANYALFNEDISVSGGGSVNFKLNASSSNIQTPGVIIKSQNFNVSGGSTLNLKAEGSTETAFSIENDLNLNATGGNITIRQVEGTDSRVNKGVAAKKNITFKGGNITFGSQKATTEIKGNVTINKNTNATLRGANFAENKSPLNIAGNVINNGNLTTAGSIINIAGNLTVSKGANLQAITNYTFNVAGSFDNNGASNISIARGGAKFKDINNTSSLNITTNSDTTYRTIIKGNISNKSGDLNIIDKKSDAEIQIGGNISQKEGNLTISSDKVNITNQITIKAGVEGGRSDSSEAENANLTIQTKELKLAGDLNISGFNKAEITAKNGSDLTIGNASGGNADAKKVTFDKVKDSKISTDGHNVTLNSEVKTSNGSSNAGNDNSTGLTISAKDVTVNNNVTSHKTINISAAAGNVTTKEGTTINATTGSVEVTAQNGTIKGNITSQNVTVTATENLVTTENAVINATSGTVNISTKTGDIKGGIESTSGNVNITASGNTLKVSNITGQDVTVTADAGALTTTAGSTISATTGNANITTKTGDINGKVESSSGSVTLVATGATLAVGNISGNTVTITADSGKLTSTVGSTINGTNSVTTSSQSGDIEGTISGNTVNVTASTGDLTIGNSAKVEAKNGAATLTAESGKLTTQTGSSITSSNGQTTLTAKDSSIAGNINAANVTLNTTGTLTTTGDSKINATSGTLTINAKDAKLDGAASGDRTVVNATNASGSGNVTAKTSSSVNITGDLNTINGLNIISENGRNTVRLRGKEIDVKYIQPGVASVEEVIEAKRVLEKVKDLSDEERETLAKLGVSAVRFVEPNNAITVNTQNEFTTKPSSQVTISEGKACFSSGNGARVCTNVADDGQQP

SEQ ID NO: 10 - protein, 1600 aa, Haemophilus influenzae
MNKIYRLKFSKRLNALVAVSELTRGCDHSTEKGSEKPVRTKVRHLALKPLSAILLSLGMASIPQSVLASGLQGMSVVHGTATMQVDGNKTTIRNSVNAIINWKQFNIDQNEMEQFLQESSNSAVFNRVTSDQISQLKGILDSNGQVFLINPNGITIGKDAIINTNGFTASTLDISNENIKARNFTLEQTKDKALAEIVNHGLITVGKDGSVNLIGGKVKNEGVISVNGGSISLLAGQKITISDIINPTITYSIAAPENEAINLGDIFAKGGNINVRAATIRNKGKLSADSVSKDKSGNIVLSAKEGEAEIGGVISAQNQQAKGGKLMITGDKVTLKTGAVIDLSGKEGGETYLGGDERGEGKNGIQLAKKTTLEKGSTINVSGKEKGGRAIVWGDIALIDGNINAQGSDIAKTGGFVETSGHDLSIGDDVIVDAKEWLLDPDDVSIETLTSGRNNTGENQGYTTGDGTKESPKGNSISKPTLTNSTLEQILRRGSYVNITANNRIYVNSSINLSNGSLTLHTKRDGVKINGDITSNENGNLTIKAGSWVDVHKNITLGTGFLNIVAGDSVAFEREGDKARNATDAQITAQGTITVNKDDKQFRFNNVSLNGTGKGLKFIANQNNFTHKFDGEINISGIVTINQTTKKDVKYWNASKDSYWNVSSLTLNTVQKFTFIKFVDSGSNGQDLRSSRRSFAGVHFNGIGGKTNFNIGANAKALFKLKPNAATDPKKELPITFNANITATGNSDSSVMFDIHANLTSRAAGINMDSINITGGLDFSITSHNRNSNAFEIKKDLTINATGSNFSLKQTKDSFYNEYSKHAINSSHNLTILGGNVTLGGENSSSSITGNINITNKANVTLQADTSNSNTGLKKRTLTLGNISVEGNLSLTGANANIVGNLSIAEDSTFKGEASDNLNITGTFTNNGTANINIKGVVKLGDINNKGGLNITTNASGTQKTIINGNITNEKGDLNIKNIKADAEIQIGGNISQKEGNLTISSDKVNITNQITIKAGVEGGRSDSSEAENANLTIQTKELKLAGDLNISGFNKAEITAKNGSDLTIGNASGGNADAKKVTFDKVKDSKISTDGHNVTLNSEVKTSNGSSNAGNDNSTGLTISAKDVTVNNNVTSHKTINISAAAGNVTTKEGTTINATTGSVEVTAQNGTIKGNITSQNVTVTATENLVTTENAVINATSGTVNISTKTGDIKGGIESTSGNVNITASGNTLKVSNITGQDVTVTADAGALTTTAGSTISATTGNANITTKTGDINGKVESSSGSVTLVATGATLAVGNISGNTVTITADSGKLTSTVGSTINGTNSVTTSSQSGDIEGTISGNTVNVTASTGDLTIGNSAKVEAKNGAATLTAESGKLTTQTGSSITSSNGQTTLTAKDSSIAGNINAANVTLNTTGTLTTTGDSKINATSGTLTINAKDAKLDGAASGDRTVVNATNASGSGNVTAKTSSSVNITGDLNTINGLNIISENGRNTVRLRGKEIDVKYIQPGVASVEEVIEAKRVLEKVKDLSDEERETLAKLGVSAVRFVEPNNAITVNTQNEFTTKPSSQVTISEGKACFSSGNGARVCTNVADDGQQP

SEQ ID NO: 11 - protein, 29 aa, Haemophilus influenzae
VDEVIEAKRILEKVKDLSDEEREALAKLG

(19) United States
(12) Patent Application Publication
(10) Pub. No.: US 2005/0053618 A1
(43) Pub. Date: Mar. 10, 2005

(54) High molecular weight surface proteins of non-typeable Haemophilus

(76) Inventor: Stephen J. Barenkamp, Webster Grove, MO (US)

Correspondence Address: SHOEMAKER AND MATTARE, LTD, 10 POST OFFICE ROAD - SUITE 110, SILVER SPRING, MD, 20910, US

(21) Appl. No.: 10/681,171

(22) Filed: Oct. 9, 2003

Related U.S. Application Data
(63) Continuation of application No. 10/092,880, filed on Apr. 29, 2002, now abandoned.
Application No. 10/092,880 is a continuation of application No. 09/155,614, filed on Sep. 30, 1998, now abandoned.

(30) Foreign Application Priority Data
Mar. 16, 1992 (GB) 9205704.1

(51) Int. Cl. G01N 33/554; G01N 33/569; C07H 21/04; A61K 39/02; C07K 14/195

(52) U.S. Cl. 424/190.1; 435/7.32; 435/69.3; 435/320.1; 435/252.3; 530/350; 536/23.7

(57) ABSTRACT

High molecular weight surface proteins of non-typeable Haemophilus influenzae which exhibit immunogenic properties and genes encoding the same are described. Specifically, genes coding for two immunodominant high molecular weight proteins, HMW1 and HMW2, have been cloned, expressed and sequenced, while genes coding for high molecular proteins HMW3 and HMW4 have also been cloned, expressed and sequenced.

### FIELD OF INVENTION

This invention relates to high molecular weight proteins of non-typeable haemophilus.

### BACKGROUND TO THE INVENTION

Non-typeable Haemophilus influenzae are non-encapsulated organisms that are defined by their lack of reactivity with antisera against known H. influenzae capsular antigens.

These organisms commonly inhabit the upper respiratory tract of humans and are frequently responsible for a variety of common mucosal surface infections, such as otitis media, sinusitis, conjunctivitis, chronic bronchitis and pneumonia. Otitis media remains an important health problem for children and most children have had at least one episode of otitis by their third birthday and approximately one-third of children have had three or more episodes. Non-typeable Haemophilus influenzae generally accounts for about 20 to 25% of acute otitis media and for a larger percentage of cases of chronic otitis media with effusion.

A critical first step in the pathogenesis of these infections is colonization of the respiratory tract mucosa. Bacterial surface molecules which mediate adherence, therefore, are of particular interest as possible vaccine candidates.

Since the non-typeable organisms do not have a polysaccharide capsule, they are not controlled by the present Haemophilus influenzae type b (Hib) vaccines, which are directed towards Hib bacterial capsular polysaccharides. The non-typeable strains, however, do produce surface antigens that can elicit bactericidal antibodies. Two of the major outer membrane proteins, P2 and P6, have been identified as targets of human serum bactericidal activity. However, it has been shown that the P2 protein sequence is variable, in particular in the non-typeable Haemophilus strains. Thus, a P2-based vaccine would not protect against all strains of the organism.

There have previously been identified by Barenkamp et al (Pediatr. Infect. Dis. J., 9:333-339, 1990) a group of high-molecular-weight (HMW) proteins of non-typeable Haemophilus influenzae that appeared to be major targets of antibodies present in human convalescent sera. Examination of a series of middle ear isolates revealed the presence of one or two such proteins in most strains. However, prior to the present invention, the structures of these proteins and their encoding nucleic acid sequences were unknown as were pure isolates of such proteins. In addition, the identification of surface accessible epitopes of such proteins was unknown.

### SUMMARY OF INVENTION

The inventor, in an effort to further characterize the high molecular weight (HMW) non-typeable Haemophilus proteins, has cloned, expressed and sequenced the genes coding for two immunodominant HMW proteins (designated HMW1 and HMW2) from a prototype non-typeable Haemophilus strain and has cloned, expressed and sequenced the genes coding for two additional immunodominant HMW proteins (designated HMW3 and HMW4) from another non-typeable Haemophilus strain.

In accordance with one aspect of the present invention, therefore, there is provided an isolated and purified nucleic acid molecule coding for a high molecular weight protein of a non-typeable Haemophilus strain, particularly a nucleic acid molecule coding for protein HMW1, HMW2, HMW3 or HMW4, as well as any variant or fragment of such protein which retains the immunological ability to protect against disease caused by a non-typeable Haemophilus strain.

The nucleic acid molecule may have a DNA sequence shown in FIG. 1 (SEQ ID No: 1) and encoding HMW1 for strain 12 having the derived amino acid sequence of FIG. 2 (SEQ ID No: 2). The nucleic acid molecule may have the DNA sequence shown in FIG. 3 (SEQ ID No: 3) and encoding protein HMW2 for strain 12 having the derived amino acid sequence of FIG. 4 (SEQ ID No: 4). The nucleic acid molecule may have the DNA sequence shown in FIG. 8 (SEQ ID No: 7) and encoding HMW3 for strain 5 having the derived amino acid sequence of FIG. 10 (SEQ ID No: 9). The nucleic acid molecule may have a DNA sequence shown in FIG. 9 (SEQ ID No: 8) and encoding protein HMW4 for strain 5 having the derived amino acid sequence of FIG. 10 (SEQ ID No: 10).

In another aspect of the invention, there is provided an isolated and purified nucleic acid molecule encoding a high molecular weight protein of a non-typeable Haemophilus strain, which is selected from the group consisting of:
(a) a DNA sequence as shown in any one of FIGS. 1, 3, 8 and 9 (SEQ ID Nos: 1, 3, 7 and 8);
(b) a DNA sequence encoding an amino acid sequence as shown in any one of FIGS. 2, 4 and 10 (SEQ ID Nos: 2, 4, 9 and 10); and
(c) a DNA sequence which hybridizes under stringent conditions to any one of the sequences of (a) and (b).
   A DNA sequence according to (c) may be one having at least about 90% identity of sequence to the DNA sequences (a) or (b).

The inventor has further found correct processing of the HMW protein requires the presence of additional downstream nucleic acid sequences. Accordingly, a further aspect of the present invention provides an isolated and purified gene cluster comprising a first nucleotide sequence encoding a high molecular weight protein of a non-typeable Haemophilus strain and at least one downstream nucleotide sequence for effecting expression of a gene product of the first nucleotide sequence fully encoded by the structural gene.

The gene cluster may comprise a DNA sequence encoding-high molecular weight protein HMW1 or HMW2 and two downstream accessory genes. The gene cluster may have the DNA sequence shown in FIG. 6 (SEQ ID No: 5) or FIG. 7 (SEQ ID No. 6).

In an additional aspect, the present invention includes a vector adapted for transformation of a host, comprising a nucleic acid molecule as provided herein, particularly the gene cluster provided herein. The vector may be an expression vector or a plasmid adapted for expression of the encoded high molecular weight protein, fragments or analogs thereof, in a heterologous or homologous host and comprising expression means operatively coupled to the nucleic acid molecule. The expression means may include a nucleic acid portion encoding a leader sequence for secretion from the host of the high molecular weight protein. The expression means may include a nucleic acid portion encoding a lipidation signal for expression from the host of a lipidated form of the high molecular weight protein. The host may be selected from, for example, E. coli, Bacillus, Haemophilus, fungi, yeast, baculovirus and Semliki Forest Virus expression systems. The invention further includes a recombinant high molecular weight protein of non-typeable Haemophilus or fragment or analog thereof producible by the transformed host.

In another aspect, the invention provides an isolated and purified high molecular weight protein of non-typeable Haemophilus influenzae which is encoded by a nucleic acid molecule as provided herein. Such high molecular weight proteins may be produced recombinantly to be devoid of non-high molecular weight proteins of non-typeable Haemophilus influenzae or from natural sources.

Such protein may be characterized by at least one surface-exposed B-cell epitope which is recognized by monoclonal antibody AD6 (ATCC ______). Such protein may be HMW1 encoded by the DNA sequence shown in FIG. 1 (SEQ ID No: 1) and having the derived amino acid sequence of FIG. 2 (SEQ ID No: 2) and having an apparent molecular weight of 125 kDa. Such protein may be HMW2 encoded by the DNA sequence shown in FIG. 3 (SEQ ID No: 3) and having the derived amino acid sequence of FIG. 4 (SEQ ID No: 4) and having an apparent molecular weight of 120 kDA. Such protein may be HMW3 encoded by the DNA sequence shown in FIG. 8 (SEQ ID No: 7) and having the derived amino acid sequence of FIG. 10 (SEQ ID No: 9) and having an apparent molecular weight of 125 kDa. Such protein may be HMW4 encoded by the DNA sequence shown in FIG. 9 (SEQ ID No: 8) and having the derived amino acid sequence shown in FIG. 10 (SEQ ID No: 10) and having the apparent molecular weight of 123 kDa.

A further aspect of the invention provides an isolated and purified high molecular weight protein of non-typeable Haemophilus influenzae which is antigenically related to the filamentous hemagglutinin surface protein of Bordetella pertussis, particularly HMW1, HMW2, HMW3 or HMW4.

The novel high molecular weight proteins of non-typeable Haemophilus may be used as carrier molecules by linking to an antigen, hapten or polysaccharide for eliciting an immune response to the antigen, hapten or polysaccharide. An example of such polysaccharide is a protective polysaccharide against Haemophilus influenzae type b.

In a further aspect of the invention, there is provided a synthetic peptide having an amino acid sequence containing at least six amino acids and no more than 150 amino acids and corresponding to at least one protective epitope of a high molecular weight protein of non-typeable Haemophilus influenzae, specifically HMW1, HMW2, HMW3 or HMW4. The epitope may be one recognized by at least one of the monoclonal antibodies AD6 (ATCC ______) and 10C5 (ATCC ______). Specifically, the epitope may be located within 75 amino acids of the carboxy terminus of the HMW1 or HMW2 protein and recognized by the monoclonal antibody AD6.

The present invention also provides an immunogenic composition comprising an immunoeffective amount of an active component, which may be the novel high molecular weight protein or synthetic peptide provided herein, which may be formulated along with a pharmaceutically acceptable carrier therefor. The immunogenic composition may be formulated as a vaccine for in vivo administration to a host.

The immunogenic composition may be formulated as a microparticle, capsule, ISCOM or liposome preparation. The immunogenic composition may be used in combination with a targeting molecule for delivery to specific cells of the immune system or to mucosal surfaces. Some targeting molecules include vitamin B12 and fragments of bacterial toxins, as described in WO 92/17167 (Biotech Australia Pty. Ltd.), and monoclonal antibodies, as described in U.S. Pat. No. 5,194,254 (Barber et al). The immunogenic compositions of the invention (including vaccines) may further comprise at least one other immunogenic or immunostimulating material and the immunostimulating material may be at least one adjuvant.

Suitable adjuvants for use in the present invention include, (but are not limited to) aluminum phosphate, aluminum hydroxide, QS21, Quil A, derivatives and components thereof, ISCOM matrix, calcium phosphate, calcium hydroxide, zinc hydroxide, a glycolipid analog, an octadecyl ester of an amino acid, a muramyl dipeptide polyphosphazare, ISCOPRP, DC-chol, DDBA and a lipoprotein and other adjuvants to induce a Th1 response. Advantageous combinations of adjuvants are described in copending U.S. patent application Ser. No. 08/261,194 filed Jun. 16, 1994, assigned to Connaught Laboratories Limited and the disclosure of which is incorporated herein by reference.

In a further aspect of the invention, there is provided a method of generating an immune response in a host, comprising administering thereto an immuno-effective amount of the immunogenic composition as provided herein. The immune response may be a humoral or a cell-mediated immune response. Hosts in which protection against disease may be conferred include primates including humans.

The present invention additionally provides a method of producing antibodies specific for a high molecular weight protein of non-typeable Haemophilus influenzae, comprising:
(a) administering the high molecular weight protein or epitope containing peptide provided herein to at least one mouse to produce at least one immunized mouse;
(b) removing B-lymphocytes from the at least one immunized mouse;
(c) fusing the B-lymphocytes from the at least one immunized mouse with myeloma cells, thereby producing hybridomas;
(d) cloning the hybridomas;
(e) selecting clones which produce anti-high molecular weight protein antibody;
(f) culturing the anti-high molecular weight protein antibody-producing clones; and then (g) isolating anti-high molecular weight protein antibodies from the cultures.

Additional aspects of the present invention include monoclonal antibody AD6 and monoclonal antibody 10C5.

The present invention provides, in an additional aspect thereof, a method for producing an immunogenic composition, comprising administering the immunogenic composition provided herein to a first test host to determine an amount and a frequency of administration thereof to elicit a selected immune response against a high molecular weight protein of non-typeable Haemophilus influenzae; and formulating the immunogenic composition in a form suitable for administration to a second host in accordance with the determined amount and frequency of administration. The second host may be a human.

The novel envelope protein provided herein is useful in diagnostic procedures and kits for detecting antibodies to high molecular weight proteins of non-typeable Haemophilus influenzae. Further monoclonal antibodies specific for the high molecular protein or epitopes thereof are useful in diagnostic procedure and kits for detecting the presence of the high molecular weight protein.

Accordingly, a further aspect of the invention provides a method of determining the presence in a sample, of antibodies specifically reactive with a high molecular weight protein of Haemophilus influenzae comprising the steps of:
(a) contacting the sample with the high molecular weight protein or epitope-containing peptide as provided herein to produce complexes comprising the protein and any said antibodies present in the sample specifically reactive therewith; and
(b) determining production of the complexes.

In a further aspect of the invention, there is provided a method of determining the presence, in a sample, of a high molecular weight protein of Haemophilus influenzae or an epitope-containing peptide, comprising the steps of:
(a) immunizing a host with the protein or peptide as provided herein, to produce antibodies specific for the protein or peptide;
(b) contacting the sample with the antibodies to produce complexes comprising any high molecular weight protein or epitope-containing peptide present in the sample and said specific antibodies; and
(c) determining production of the complexes.

A further aspect of the invention provides a diagnostic kit for determining the presence of antibodies in a sample specifically reactive with a high molecular weight protein of non-typeable Haemophilus influenzae or epitope-containing peptide, comprising:
(a) the high molecular weight protein or epitope-containing peptide as provided herein;
(b) means for contacting the protein or peptide with the sample to produce complexes comprising the protein or peptide and any said antibodies present in the sample; and
(c) means for determining production of the complexes.

The invention also provides a diagnostic kit for detecting the presence, in a sample, of a high molecular weight protein of Haemophilus influenzae or epitope-containing peptide, comprising:
(a) an antibody specific for the novel envelope protein as provided herein;
(b) means for contacting the antibody with the sample to produce a complex comprising the protein or peptide and protein-specific antibody; and
(c) means for determining production of the complex.

In this application, the term “high molecular weight protein” is used to define a family of high molecular weight proteins of Haemophilus influenzae, generally having an apparent molecular weight of from about 120 to about 130 kDa and includes proteins having variations in their amino acid sequences. In this application, a first protein or peptide is a “functional analog” of a second protein or peptide if the first protein or peptide is immunologically related to and/or has the same function as the second protein or peptide. The functional analog may be, for example, a fragment of the protein or a substitution, addition or deletion mutant thereof. The invention also extends to such functional analogs.

Advantages of the present invention include:
an isolated and purified envelope high molecular weight protein of Haemophilus influenzae produced recombinantly to be devoid of non-high molecular weight proteins of Haemophilus influenzae or from natural sources as well as nucleic acid molecules encoding the same;
high molecular weight protein specific human monoclonal antibodies which recognize conserved epitopes in such protein; and
diagnostic kits and immunological reagents for specific identification of hosts infected by Haemophilus influenzae.

### BRIEF DESCRIPTION OF DRAWINGS

FIGS. 1A to 1G contain the DNA sequence of a gene coding for protein HMW1 (SEQ ID No: 1). The hmw1A open reading frame extends from nucleotides 351 to 4958;

FIGS. 2A and 2B contain the derived amino acid sequence of protein HMW1 (SEQ ID No: 2);

FIGS. 3A to 3G contain the DNA sequence of a gene coding for protein HMW2 (SEQ ID No: 3). The open hmw2A open reading frame extends from nucleotides 382 to 4782;

FIGS. 4A and 4B contain the derived amino acid sequence of HMW2 (SEQ ID No: 4);

FIG. 5A shows restriction maps of representative recombinant phages which contained the HMW1 or HMW2 structural genes and of HMW1 plasmid subclones. The shaded boxes indicate the location of the structural genes. In the recombinant phage, transcription proceeds from left to right for the HMW1 gene and from right to left for the HMW2 gene;

FIG. 5B shows the restriction map of the T7 expression vector pT7-7. This vector contains the T7 RNA polymerase promoter Φ10, a ribosomal binding site (rbs) and the translational start site for the T7 gene 10 protein upstream from a multiple cloning site;

FIGS. 6A to 6L contain the DNA sequence of a gene cluster for the hmw1 gene (SEQ ID NO: 5), comprising nucleotides 351 to 4958 (ORF a) (as in FIG. 1), as well as two additional downstream genes in the 3′ flanking region, comprising ORFs b, nucleotides 5114 to 6748 and c nucleotides 7062 to 9011;

FIGS. 7A to 7L contain the DNA sequence of a gene cluster for the hmw2 gene (SEQ ID NO: 6), comprising nucleotides 792 to 5222 (ORF a) (as in FIG. 3), as well as two additional downstream genes in the 3′ flanking region, comprising ORFs b, nucleotides 5375 to 7009, and c, nucleotides 7249 to 9198;

FIGS. 8A and 8B contain the DNA sequence of a gene coding for protein HMW3 (SEQ ID NO: 7);

FIGS. 9A and 9B contain the DNA sequence of a gene coding for protein HMW4 (SEQ ID NO: 8);

FIGS. 10A to 10L contain a comparison table for the derived amino acid sequence for proteins HMW1 (SEQ ID No: 2), HMW2 (SEQ ID No: 4), HMW3 (SEQ ID No: 9) and HMW4 (SEQ ID No: 10);

FIG. 11 illustrates a Western immunoblot assay of phage lysates containing either the HMW1 or HMW2 recombinant proteins. Lysates were probed with an E. coli-absorbed adult serum sample with high-titer antibody against high molecular weight proteins. The arrows indicate the major immunoreactive bands of 125 and 120 kDa in the HMW1 and HMW2 lysates respectively;

FIG. 12 is a Western immunoblot assay of cell sonicates prepared from E. coli transformed with plasmid pT7-7 (lanes 1 and 2), pHMW1-2 (lanes 3 and 4), pHMW1-4 (lanes 5 and 6) or pHMW1-14 (lanes 7 and 8). The sonicates were probed with an E. coli-absorbed adult serum sample with high-titer antibody against high-molecular weight proteins. Lanes labelled U and I sequence sonicates prepared before and after indication of the growing samples with IPTG, respectively. The arrows indicate protein bands of interest as discussed below;

FIG. 13 is a graphical illustration of an ELISA with rHMW1 antiserum assayed against purified filamentous haemagglutinin of B. pertussis. Ab=antibody;

FIG. 14 is a Western immunoblot assay of cell sonicates from a panel of epidemiologically unrelated non-typeable H. influenzae strains. The sonicates were probed with rabbit antiserum prepared against HMW1-4 recombinant protein. The strain designations are indicated by the numbers below each line;

FIG. 15 is a Western immunoblot assay of cell sonicates from a panel of epidemiologically unrelated non-typeable H. influenzae strains. The sonicates were probed with monoclonal antibody X3C, a murine IgG antibody which recognizes the filamentous hemagglutinin of B. pertussis. The strain designations are indicated by the numbers below each line;

FIG. 16 shows an immunoblot assay of cell sonicates of non-typeable H. influenzae strain 12 derivatives. The sonicates were probed with rabbit antiserum prepared against HMW-1 recombinant protein. Lanes: 1, wild-type strain; 2, HMW2⁻ mutant; 3, HMW1⁻ mutant; 4. HMW1⁻ HMW2⁻ double mutant;

FIG. 17 shows middle ear bacterial counts in PBS-immunized control animals (left panel) and HMW1/HMW2-immunized animals (right panel) seven days after middle ear inoculation with non-typeable Haemophilus influenzae strain 12. Data are log-transformed and the horizontal lanes indicate the means and standard deviations of middle ear fluid bacterial counts for only the infected animals in each group;

FIG. 18 is a schematic diagram of pGEMEX®-hmw1 recombinant plasmids. The restriction enzymes are B-BamHI, E-EcoRI, C-ClaI, RV-EcoRV, Bst-BstEII and H-HindIII;

FIG. 19 is a schematic diagram of pGEMEX®-hmw2 recombinant plasmids. The restriction enzymes are E-EcoRI, H-HindIII, Hc-HincII, M-MluI and X-XhoI;

FIG. 20 is an immunoelectron micrograph of representative non-typeable Haemophilus influenzae strains after incubation with monoclonal antibody AD6 followed by incubation with goat anti-mouse IgG conjugated with 10-nm colloidal gold particles. Strains are: upper left panel-strain 12; upper right panel-strain 12 mutant deficient in expression of the high molecular weight proteins; lower left panel-strain 5; lower right panel-strain 15;

FIG. 21 is a Western immunoblot assay with Mab AD6 and HMW1 or HMW2 recombinant proteins. The upper left panel indicates the segments of hmw1A or hmw2A structural genes which are being expressed in the recombinant proteins. The lane numbers correspond to the indicated segments;

FIG. 22 is a Western immunoblot assay with MAb 10C5 and HMW1 or HMW2 recombinant proteins. The upper panel indicates the segments of the hmw1A or hmw2A structural genes which are being expressed in the recombinant proteins. The lane numbers correspond to the indicated segments; and

FIG. 23 is a Western immunoblot assay with MAb AD6 and a panel of unrelated non-typeable Haemophilus influenzae strains which express HMW1/HMW-2 like protein. Cell sonicates were prepared from freshly grown samples of each strain prior to analysis in the Western blot.

### GENERAL DESCRIPTION OF INVENTION

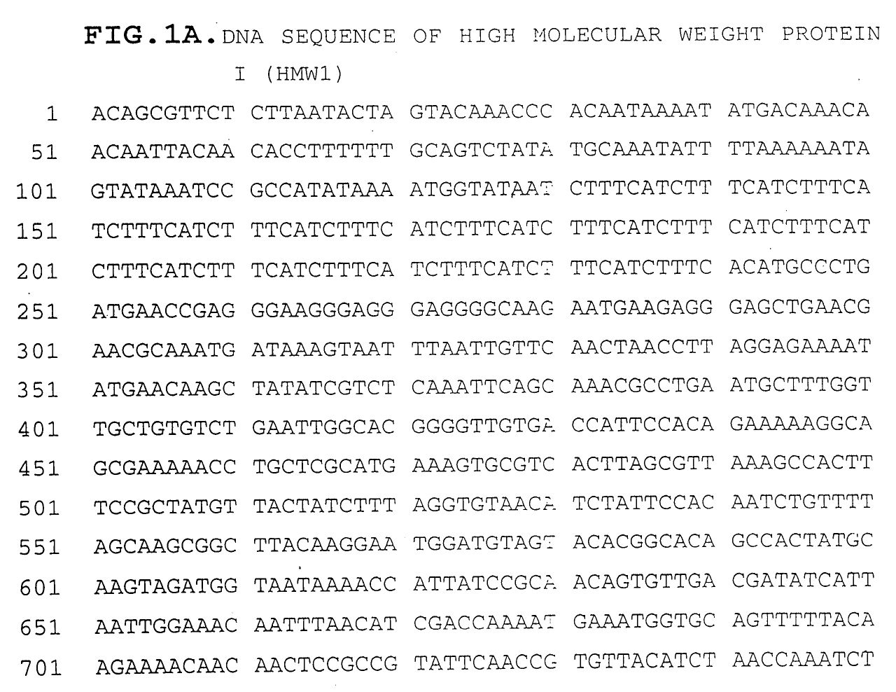

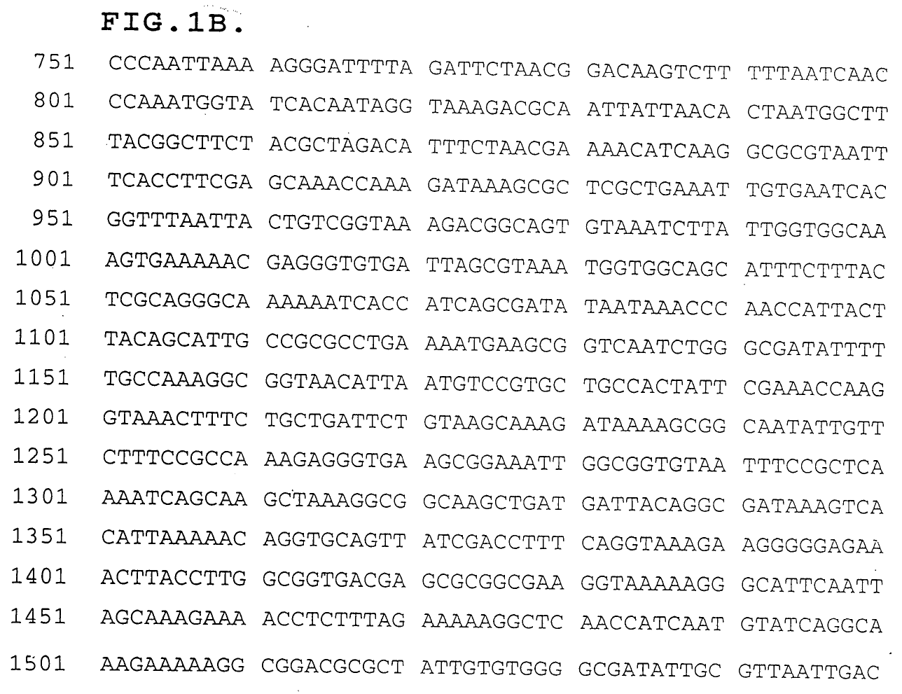

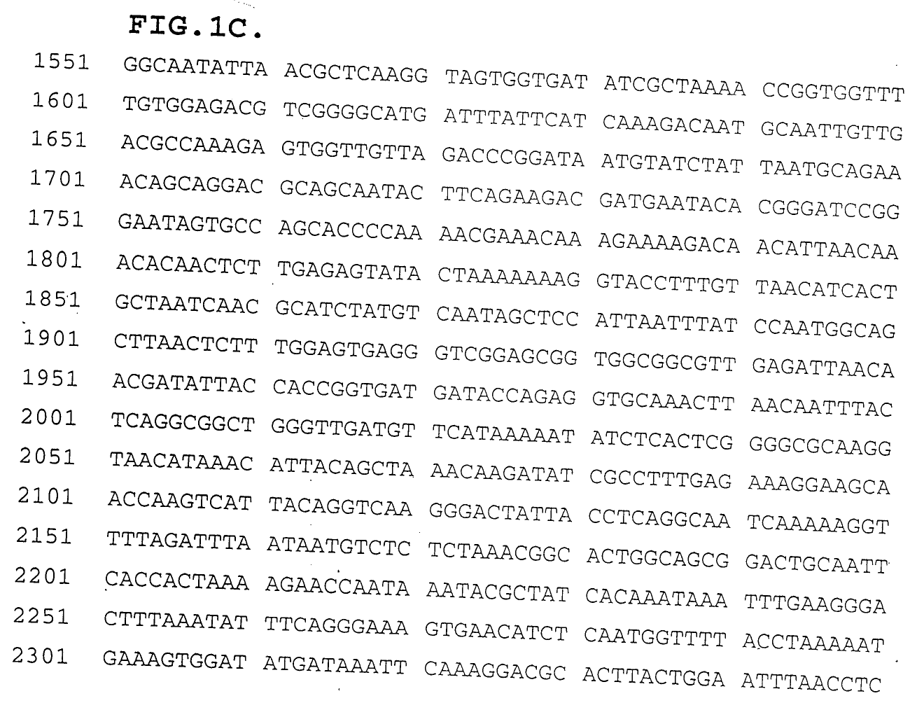

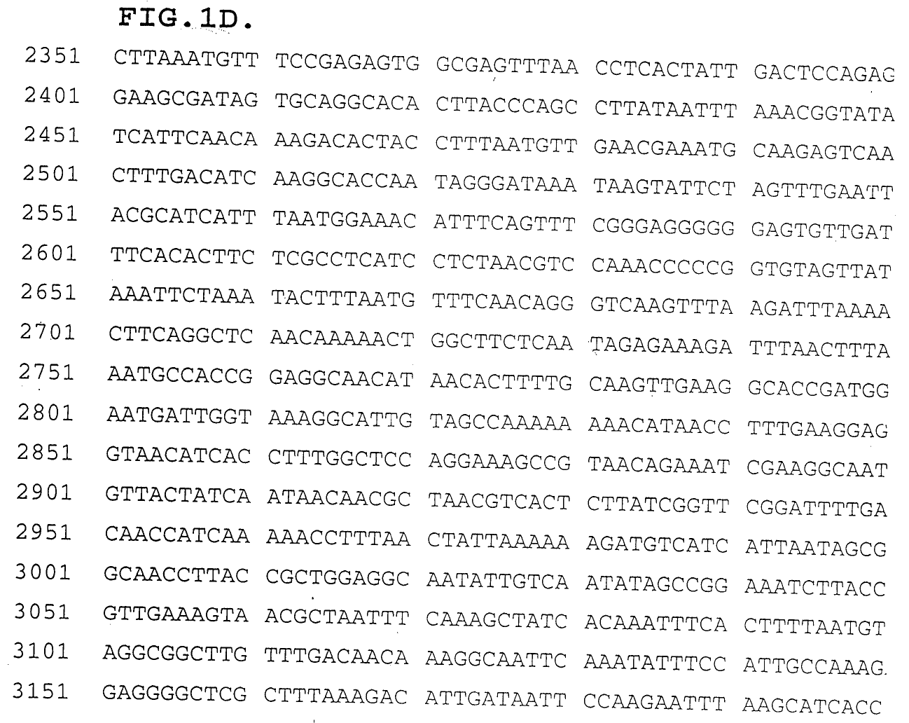

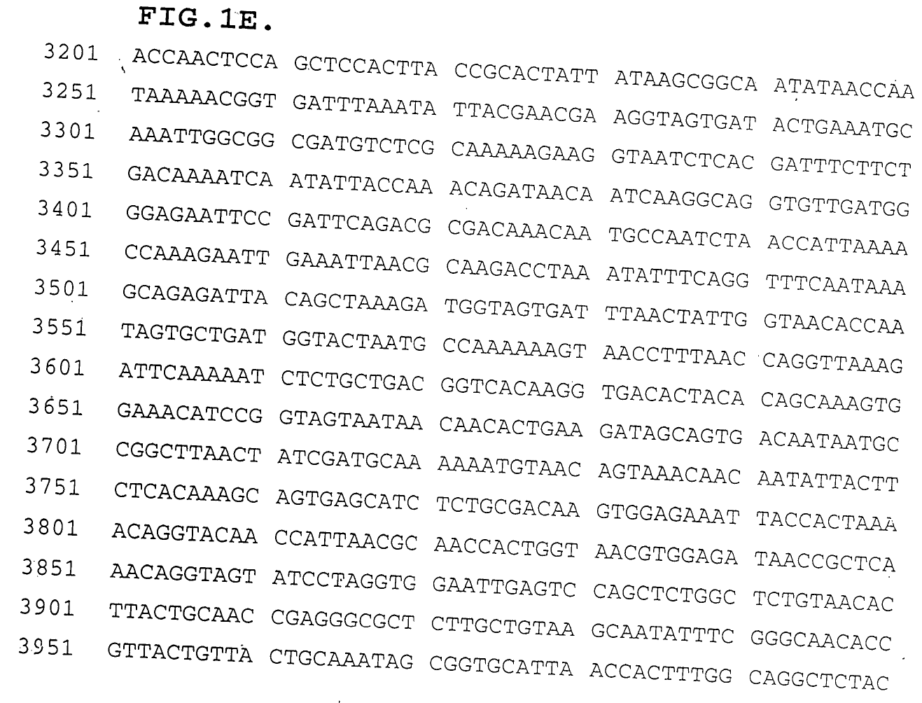

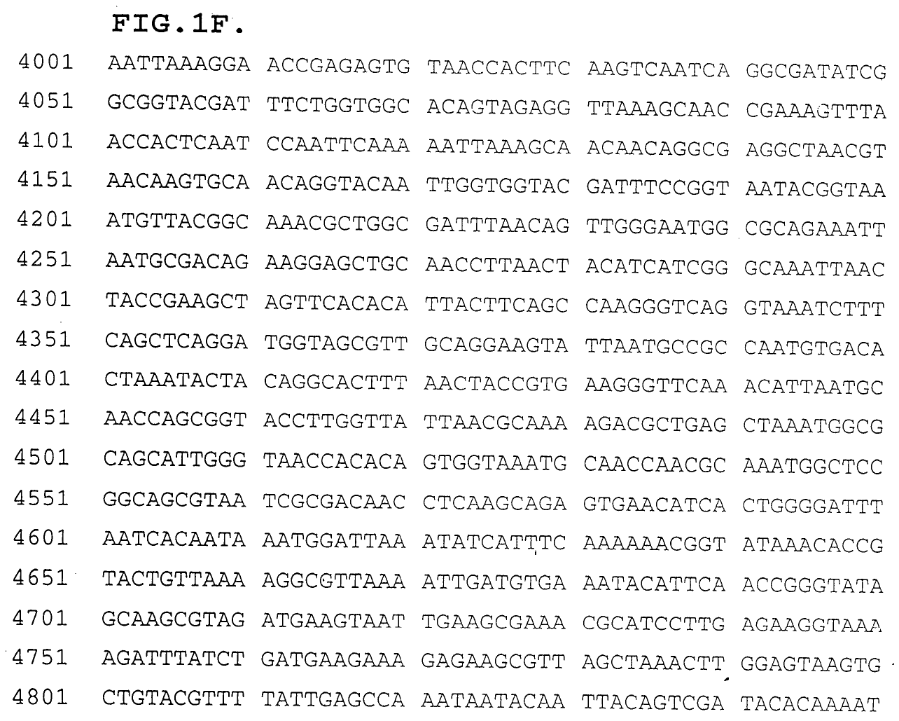

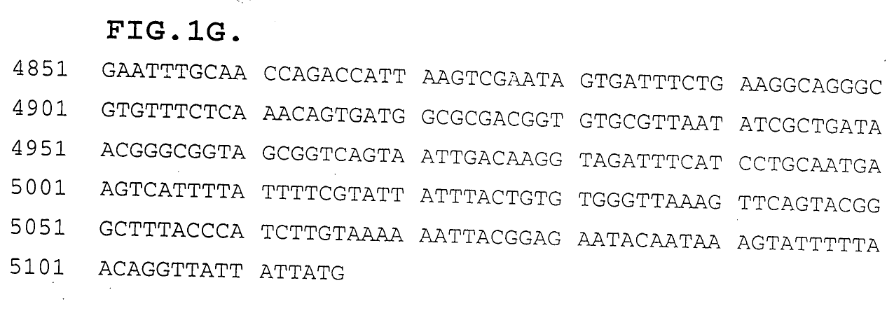

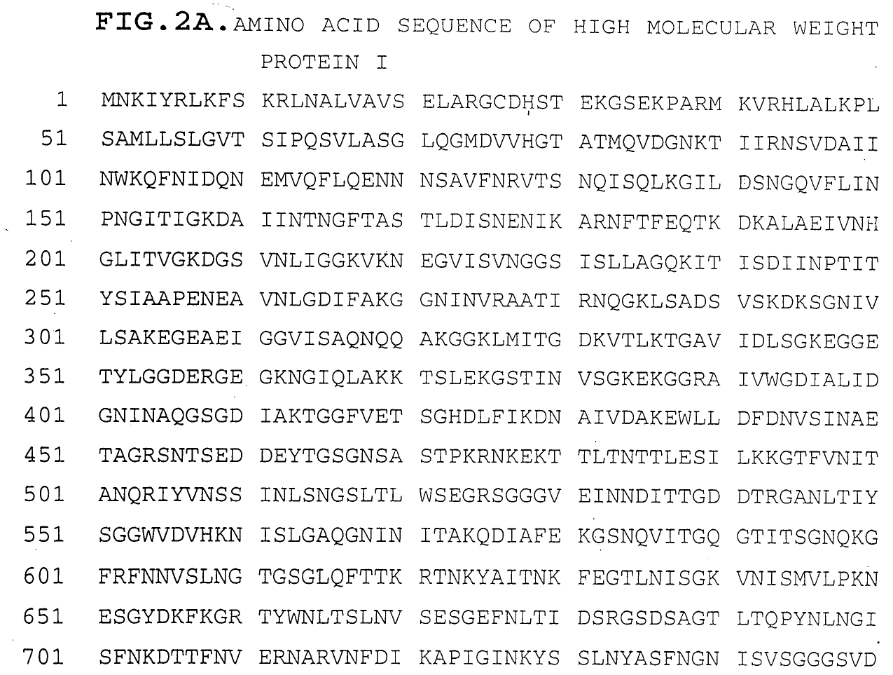

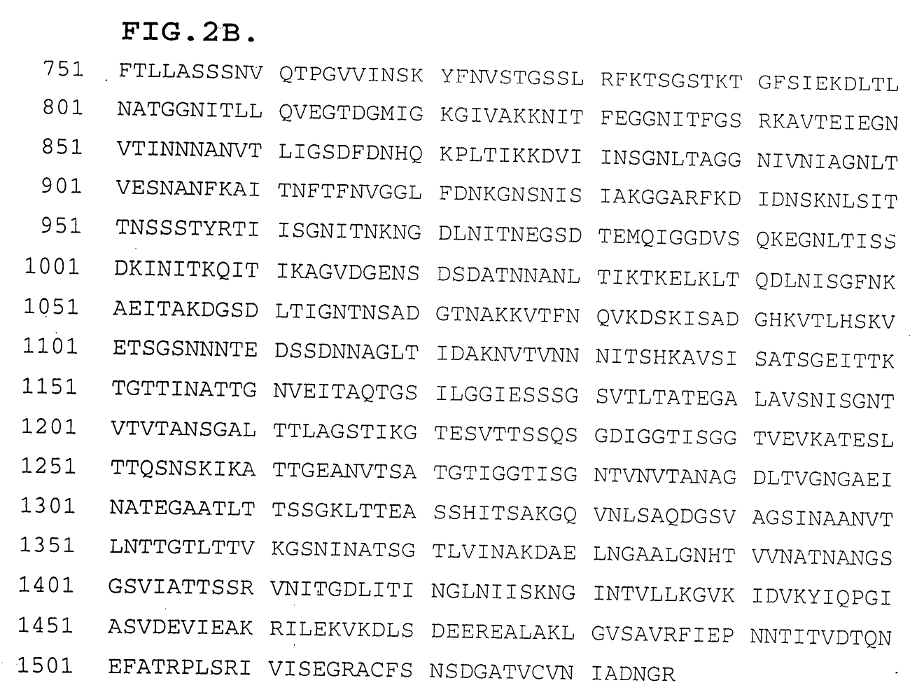

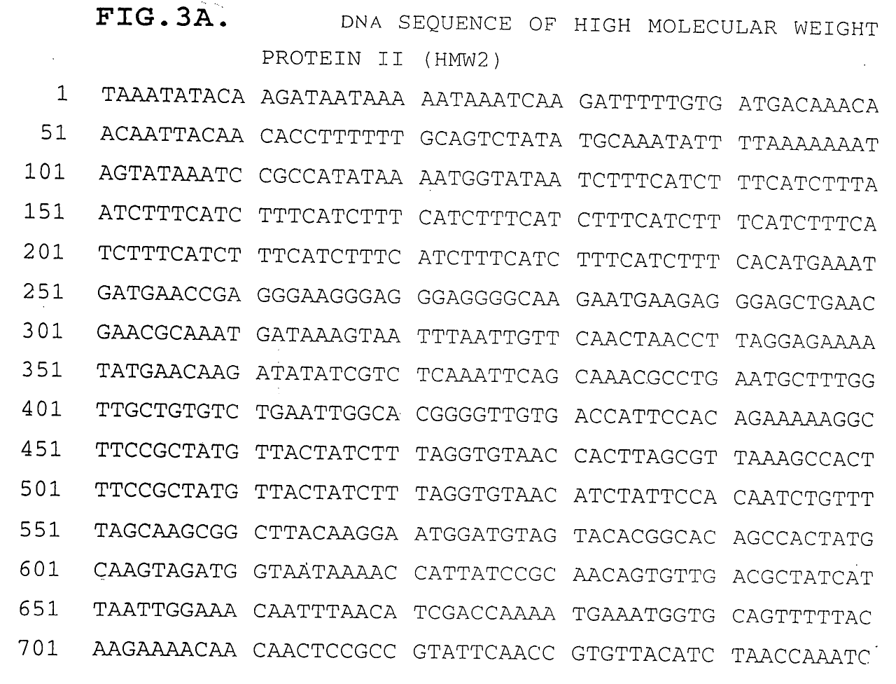

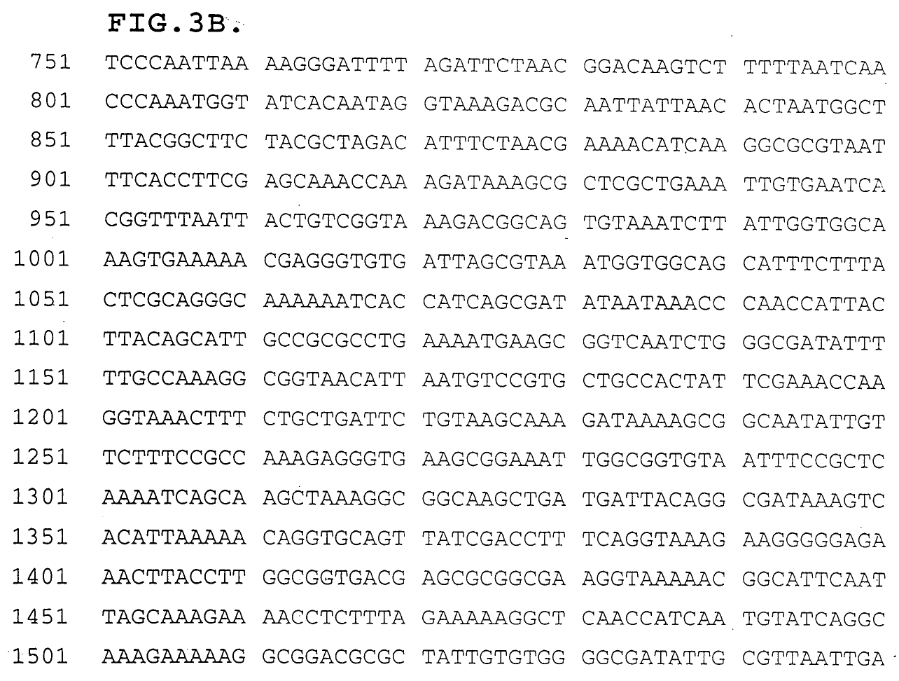

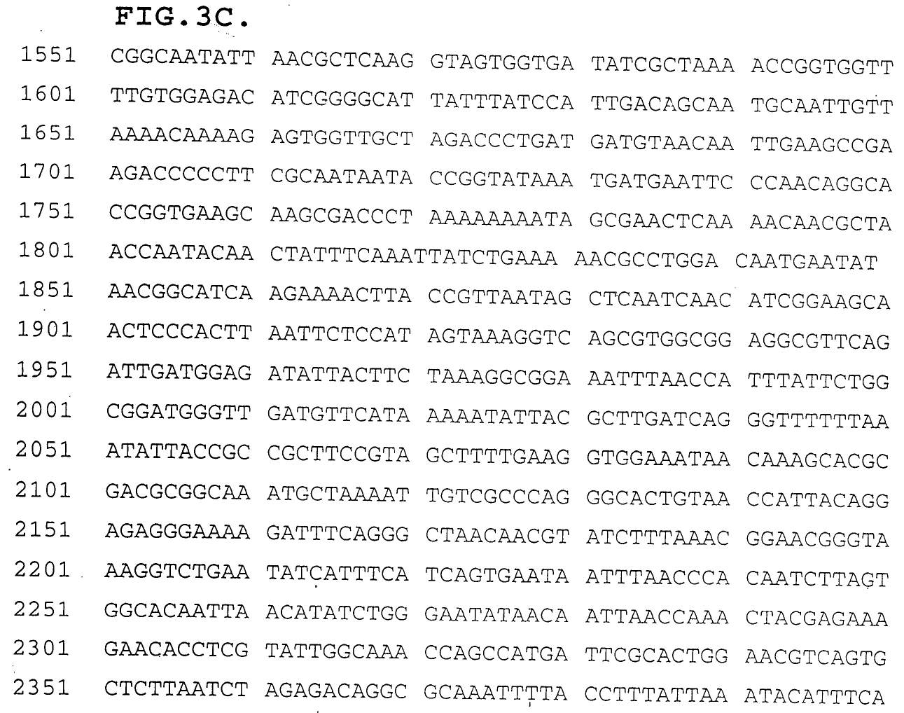

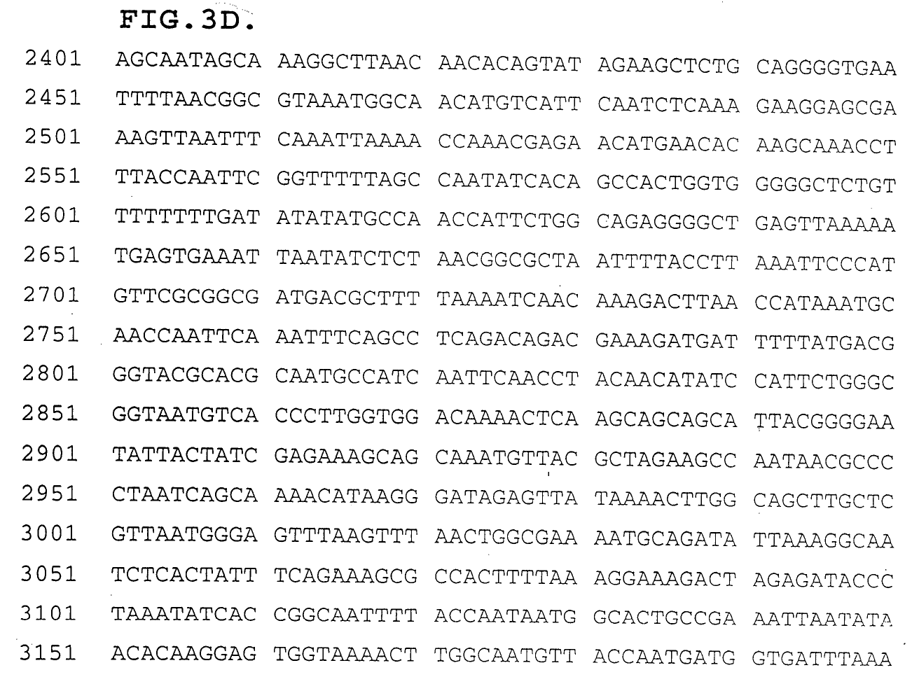

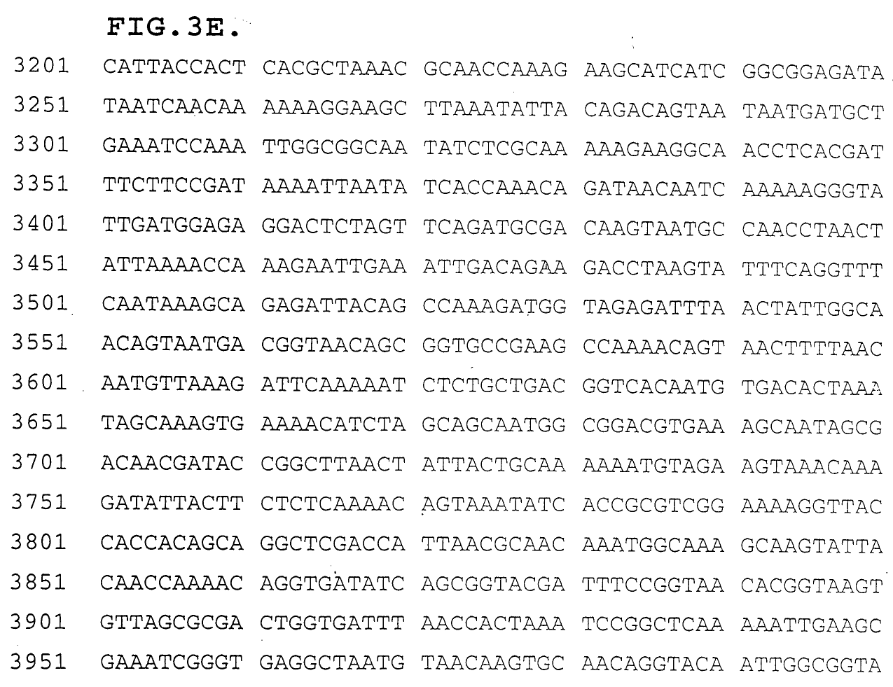

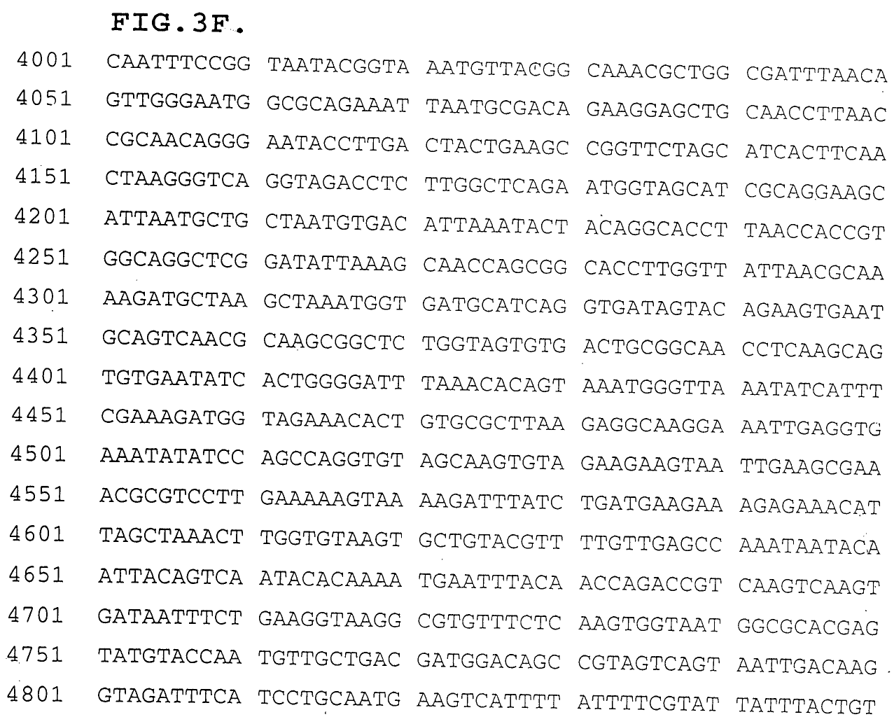

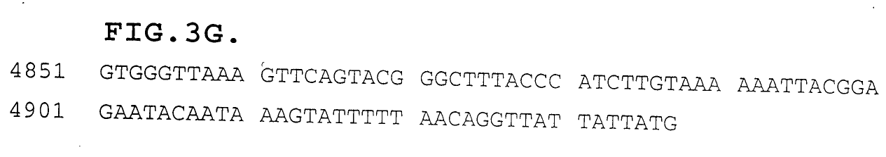

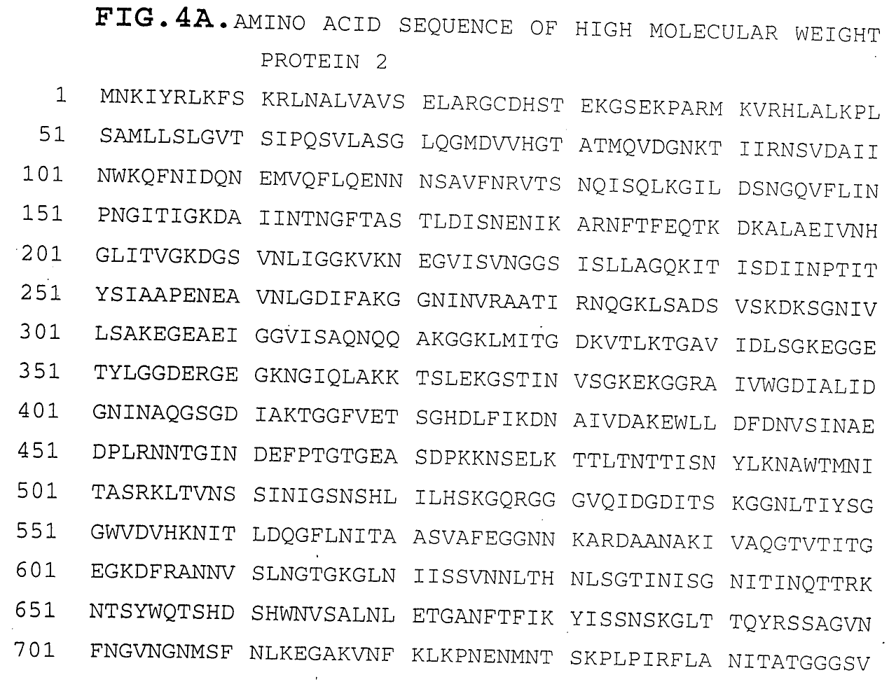

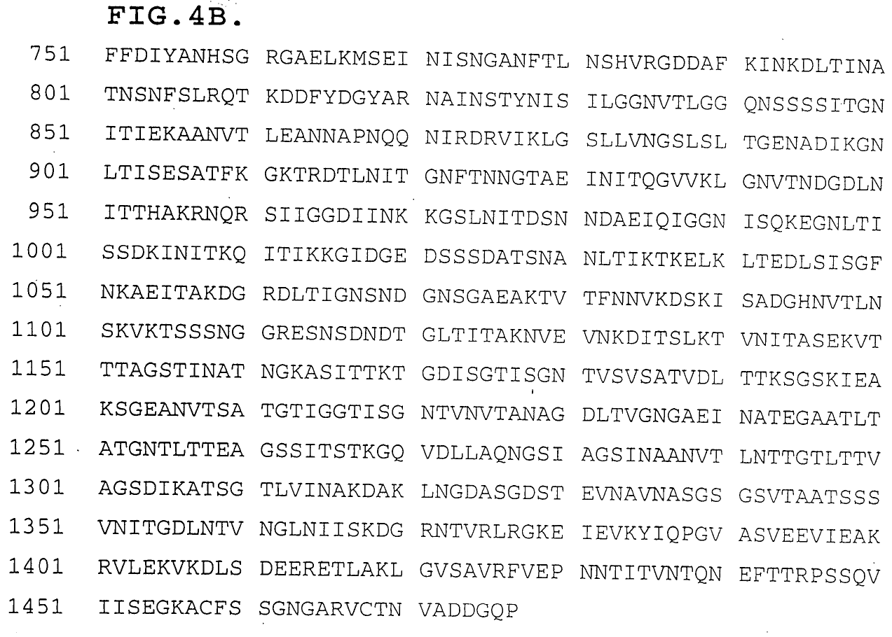

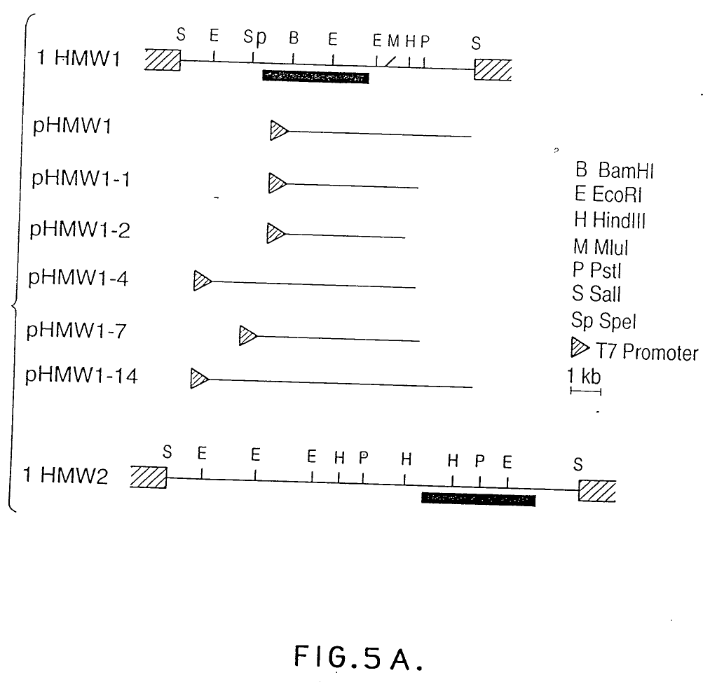

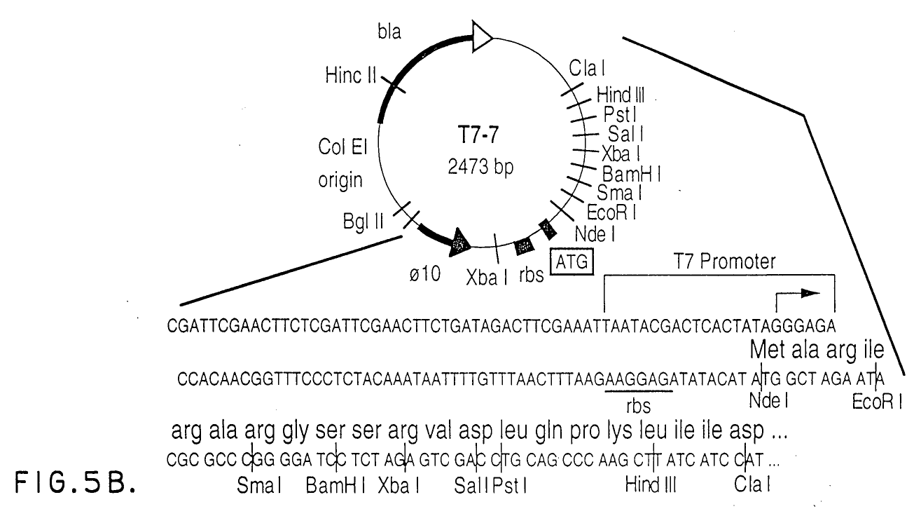

The DNA sequences of the genes coding for the HMW1 and HMW2 proteins of non-typeable Haemophilus influenzae strain 12, shown in FIGS. 1 and 3 respectively, were shown to be about 80% identical, with the first 1259 base pairs of the genes being identical. The open reading frame extend from nucleotides 351 to 4958 and from nucleotide 382 to 4782 respectively. The derived amino acid sequences of the two HMW proteins, shown in FIGS. 2 and 4 respectively, are about 70% identical. Furthermore, the encoded proteins are antigenically related to the filamentous hemagglutinin surface protein of Bordetella pertussis. A monoclonal antibody prepared against filamentous hemagglutinin (FHA) of Bordetella pertussis was found to recognize both of the high molecular weight proteins. This data suggests that the HMW and FRA proteins may serve similar biological functions. The derived amino acid sequences of the HMW1 and HMW2 proteins show sequence similarity to that for the FHA protein. It has further been shown that these antigenically-related proteins are produced by the majority of the non-typeable strains of Haemophilus. Antisera raised against the protein expressed by the HMW1 gene recognizes both the HMW2 protein and the B. pertussis FHA. The present invention includes an isolated and purified high molecular weight protein of non-typeable haemophilus which is antigenically related to the B. pertussis FHA and which may be obtained from natural sources or produced recombinantly.

A phage genomic library of a known strain of non-typeable Haemophilus was prepared by standard methods and the library was screened for clones expressing high molecular weight proteins, using a high titre antiserum against HMW's. A number of strongly reactive DNA clones were plaque-purified and sub-cloned into a T7 expression plasmid. It was found that they all expressed either one or the other of the two high-molecular-weight proteins designated HMW1 and HMW2, with apparent molecular weights of 125 and 120 kDa, respectively, encoded by open reading frames of 4.6 kb and 4.4 kb, respectively.

Representative clones expressing either HMW1 or HMW2 were further characterized and the genes isolated, purified and sequenced. The DNA sequence of HMW1 is shown in FIG. 1 and the corresponding derived amino acid sequence in FIG. 2. Similarly, the DNA sequence of HMW2 is shown in FIG. 3 and the corresponding derived amino acid sequence in FIG. 4. Partial purification of the isolated proteins and N-terminal sequence analysis indicated that the expressed proteins are truncated since their sequence starts at residue number 442 of both full length HMW1 and HMW2 gene products.

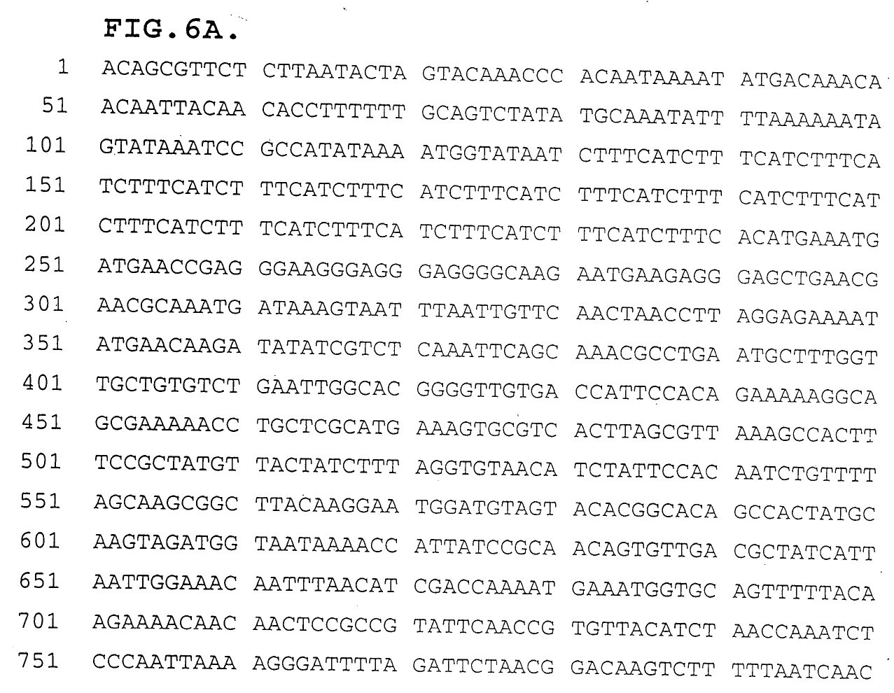

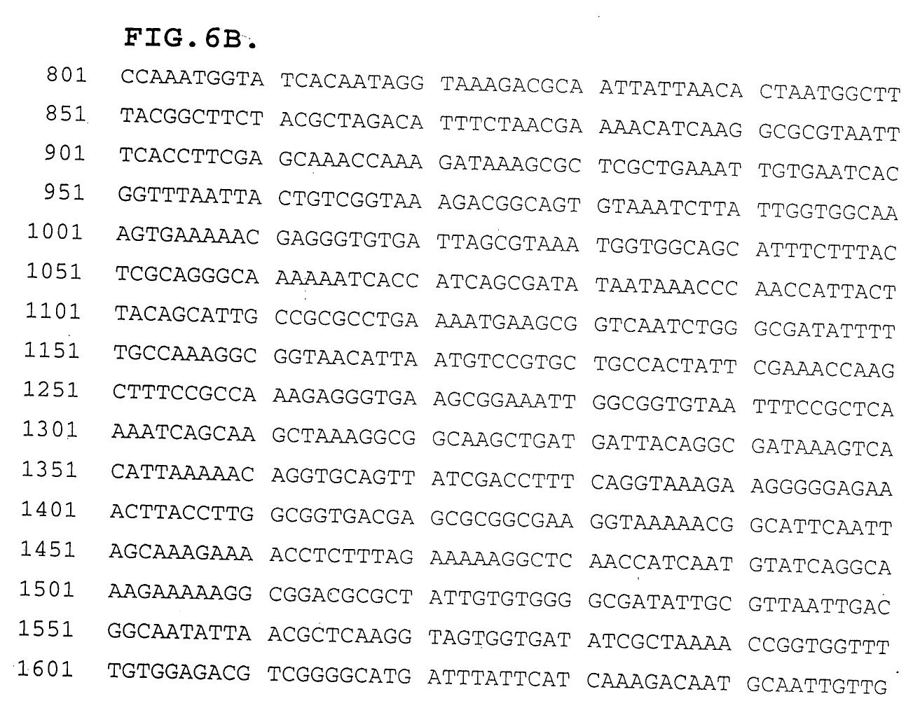

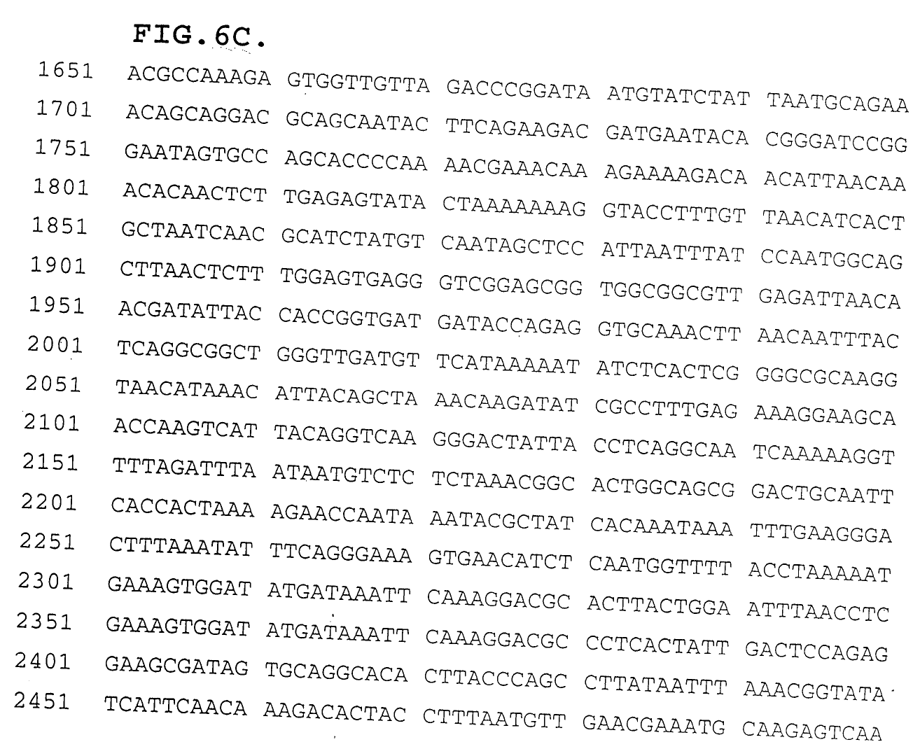

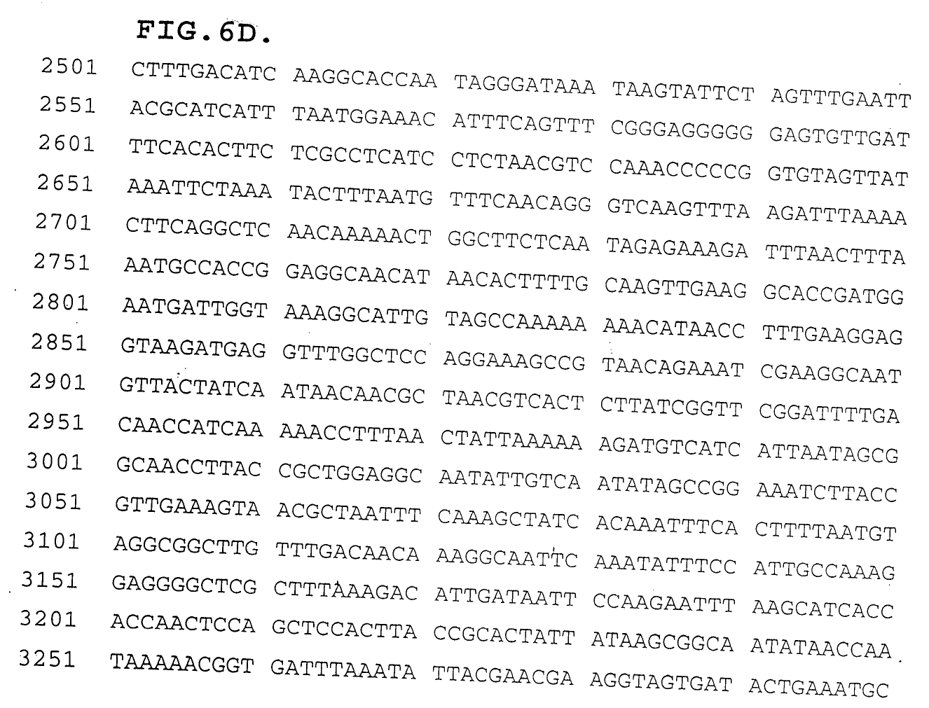

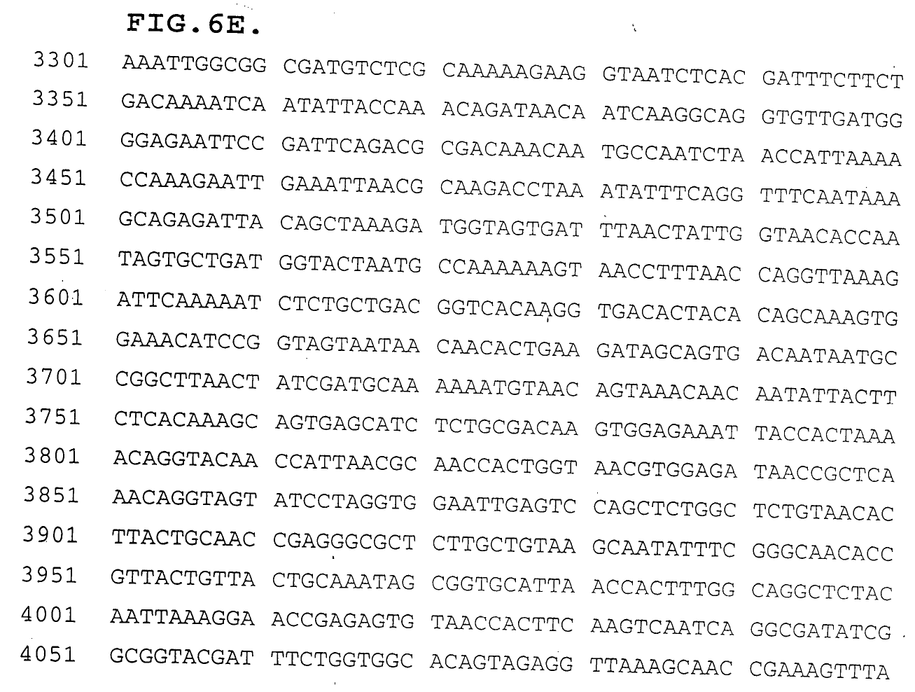

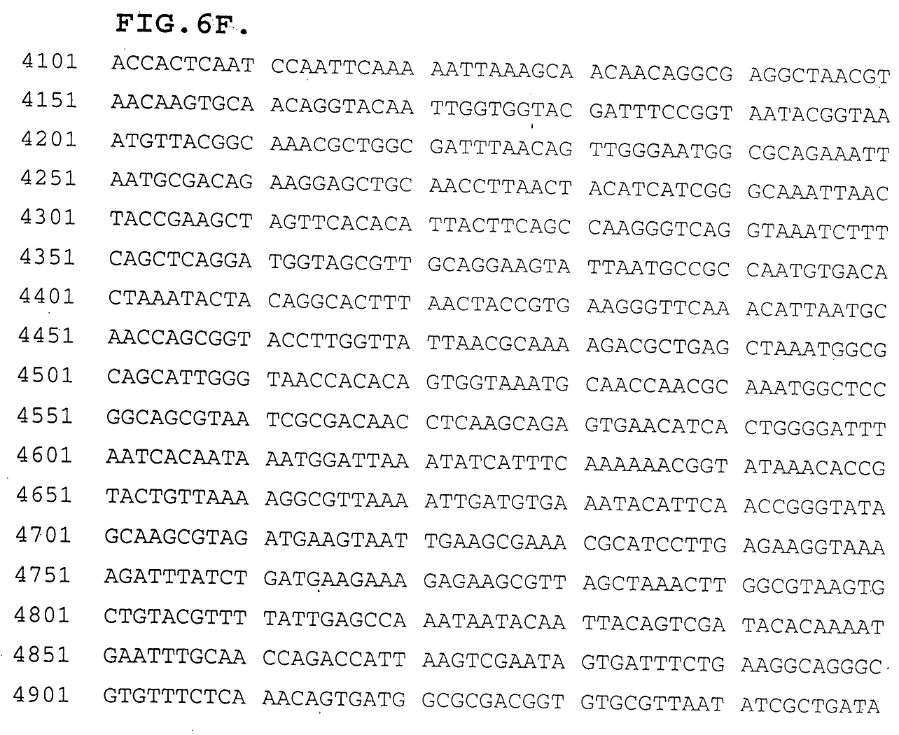

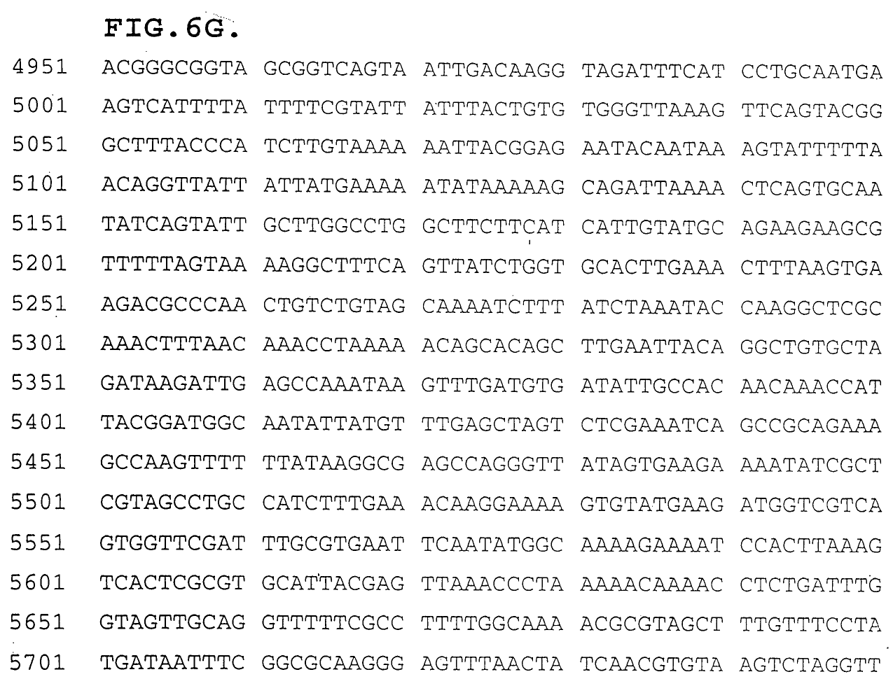

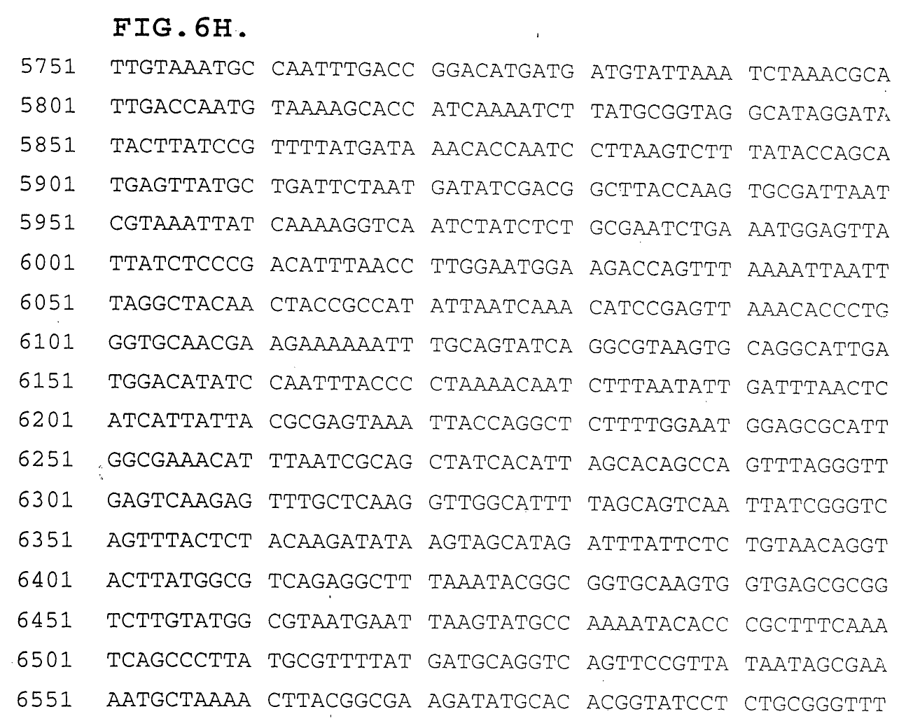

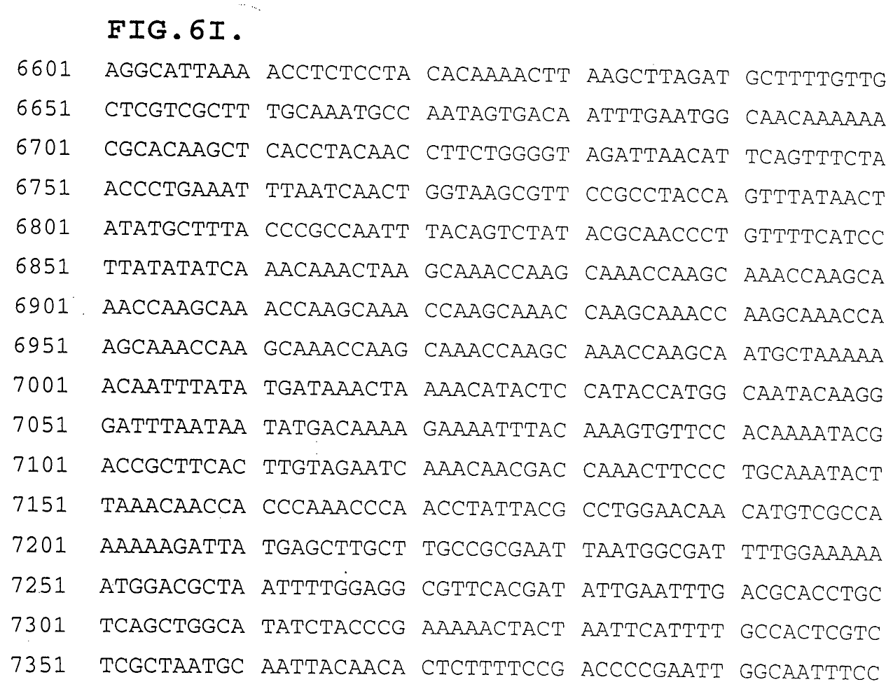

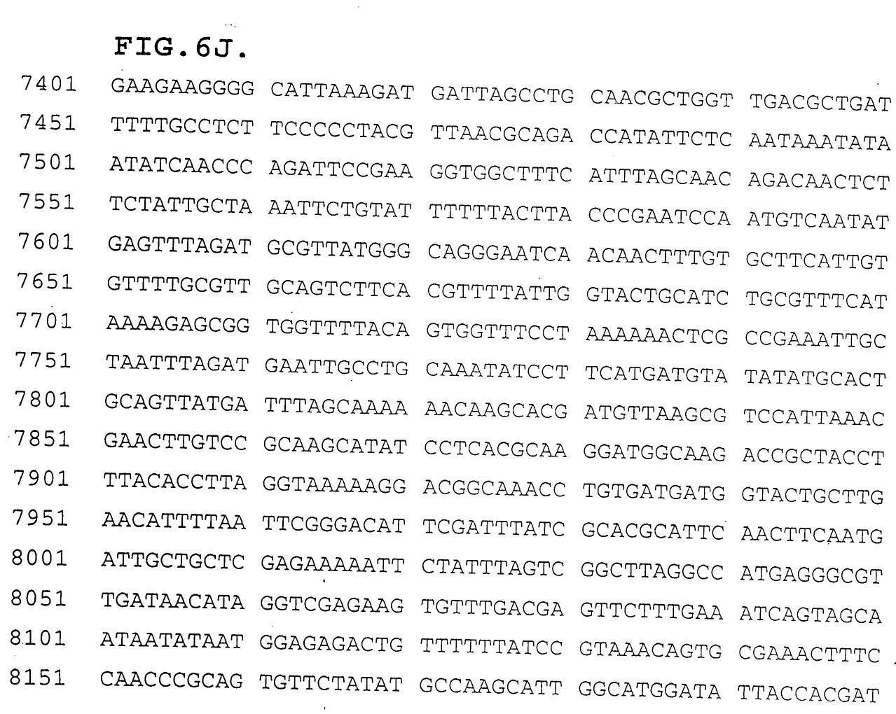

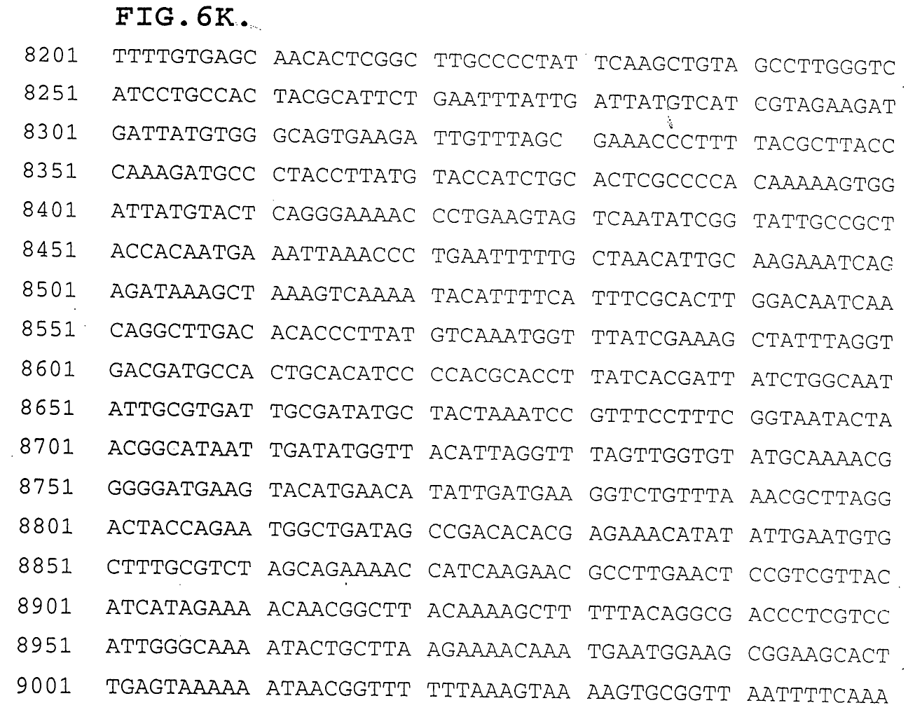

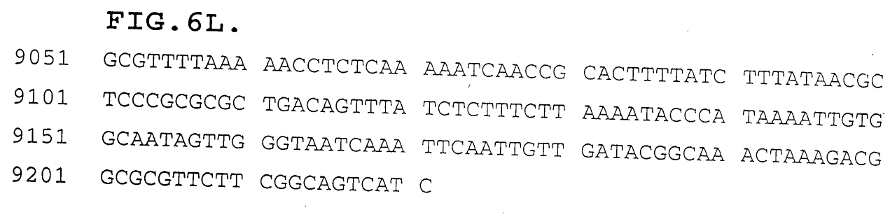

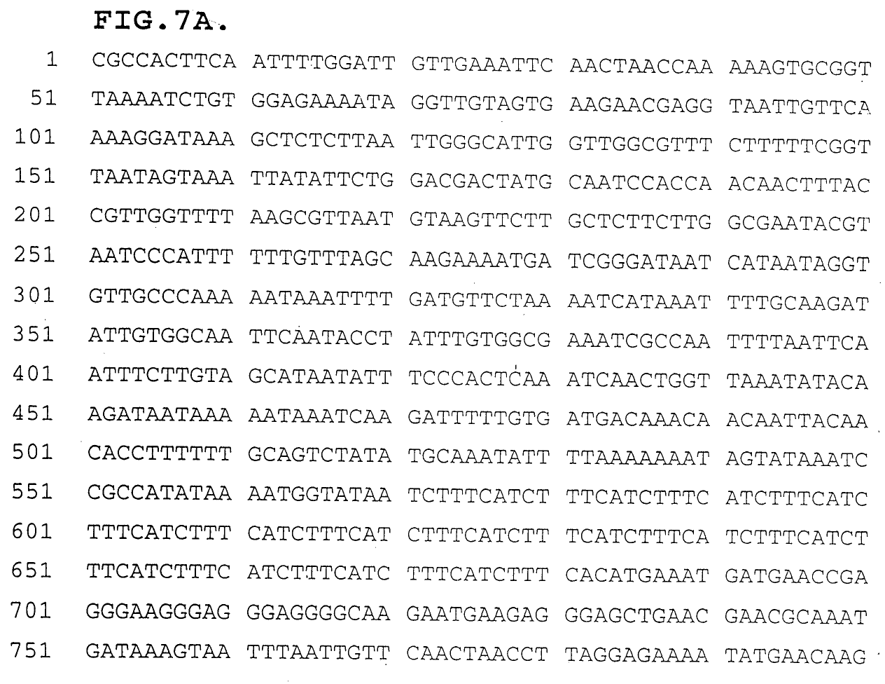

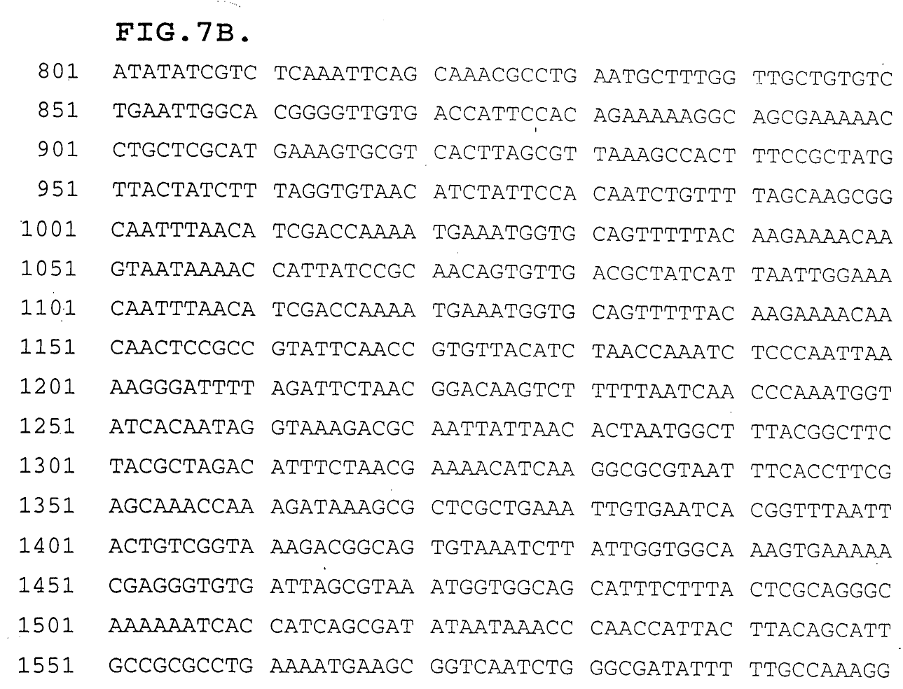

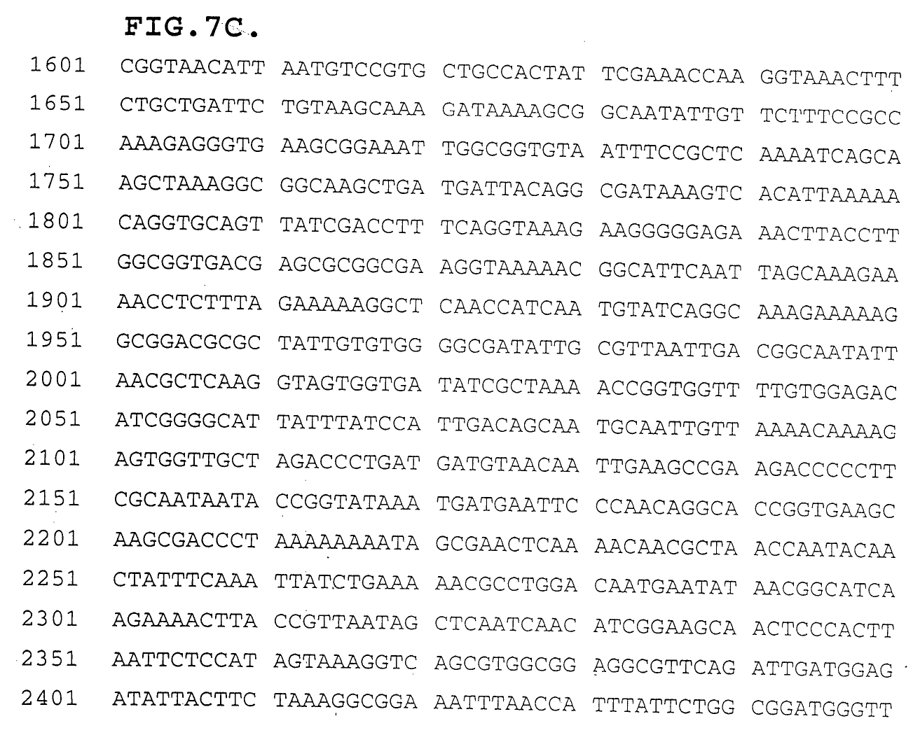

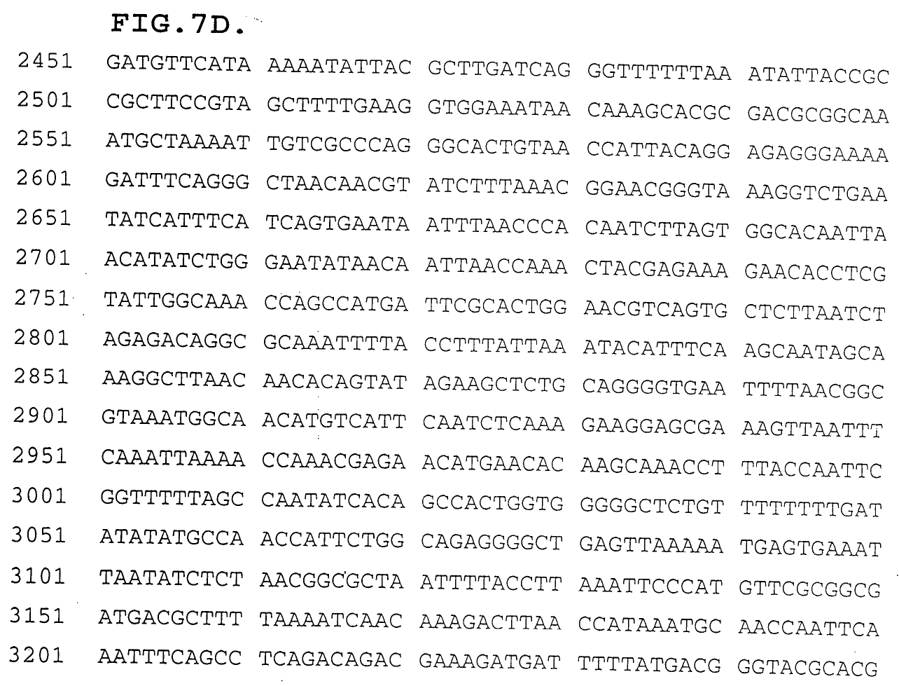

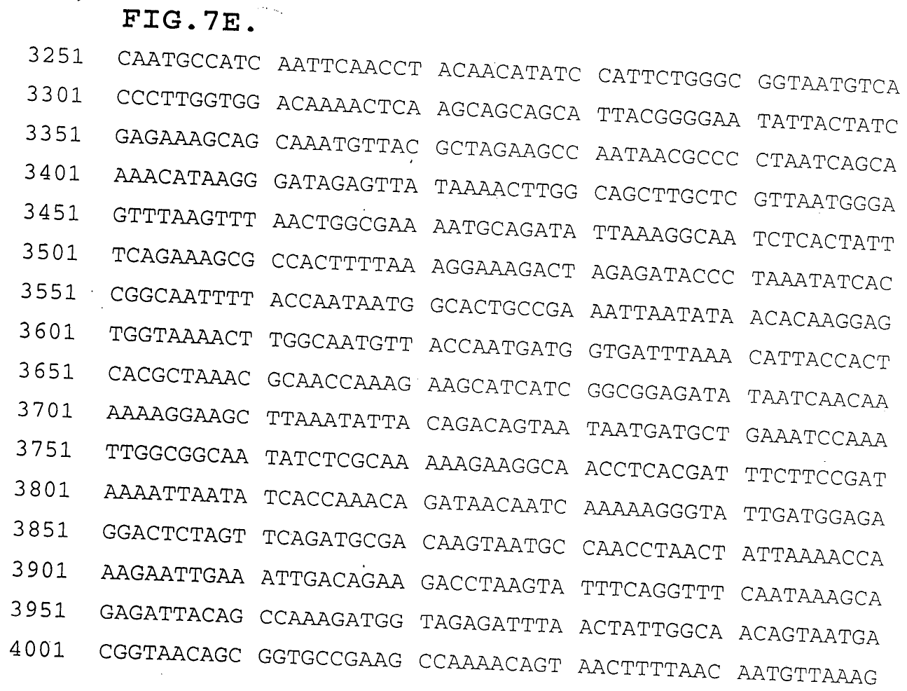

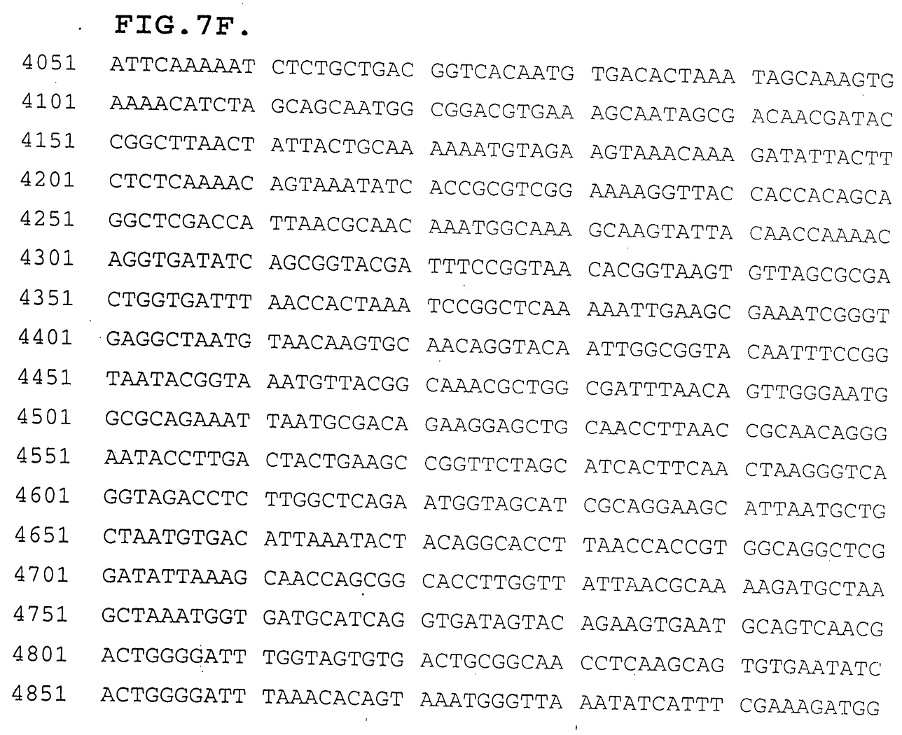

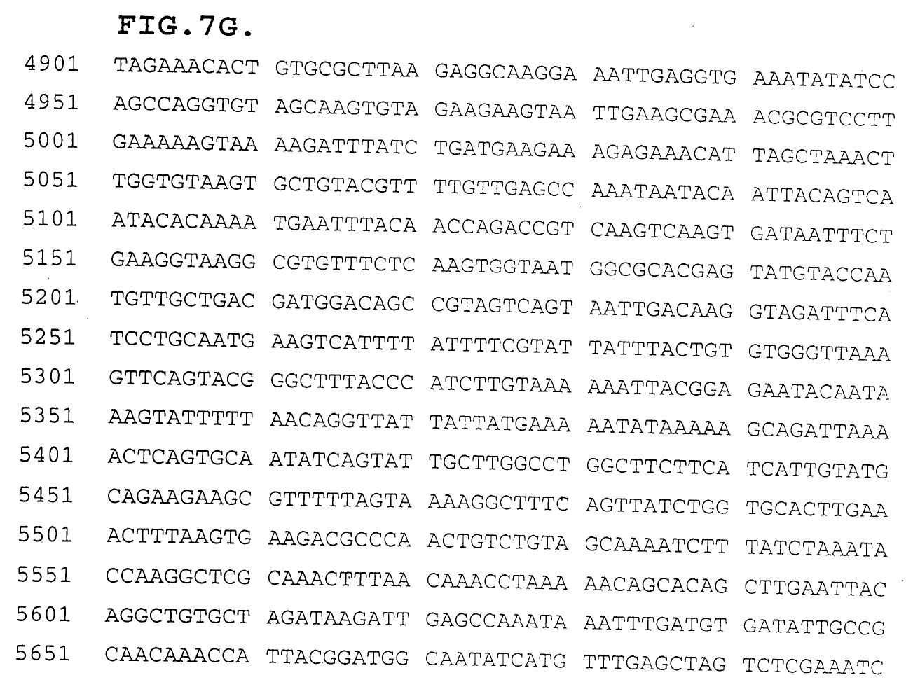

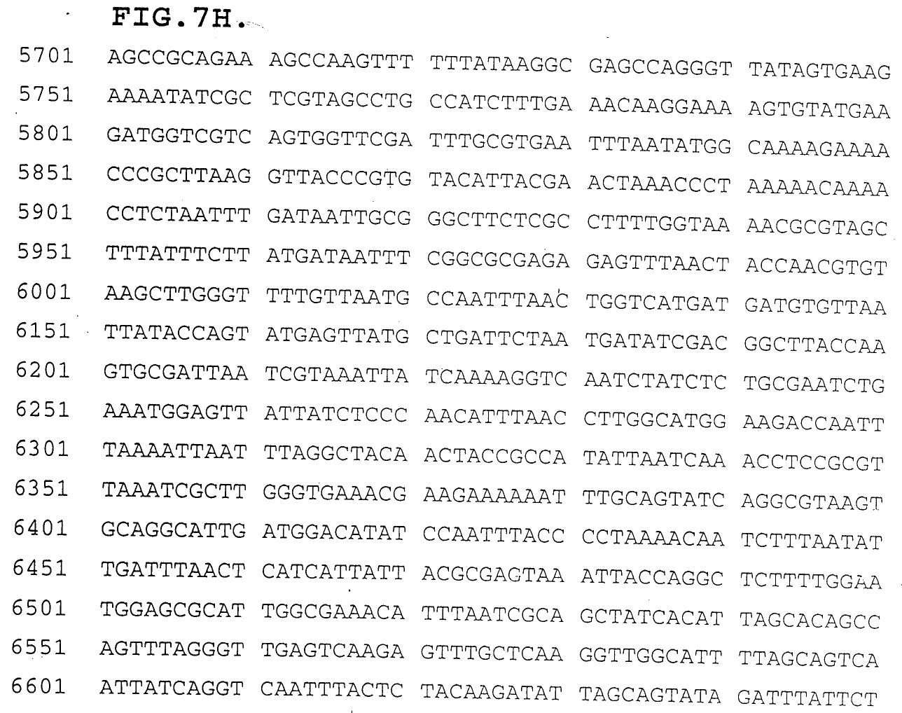

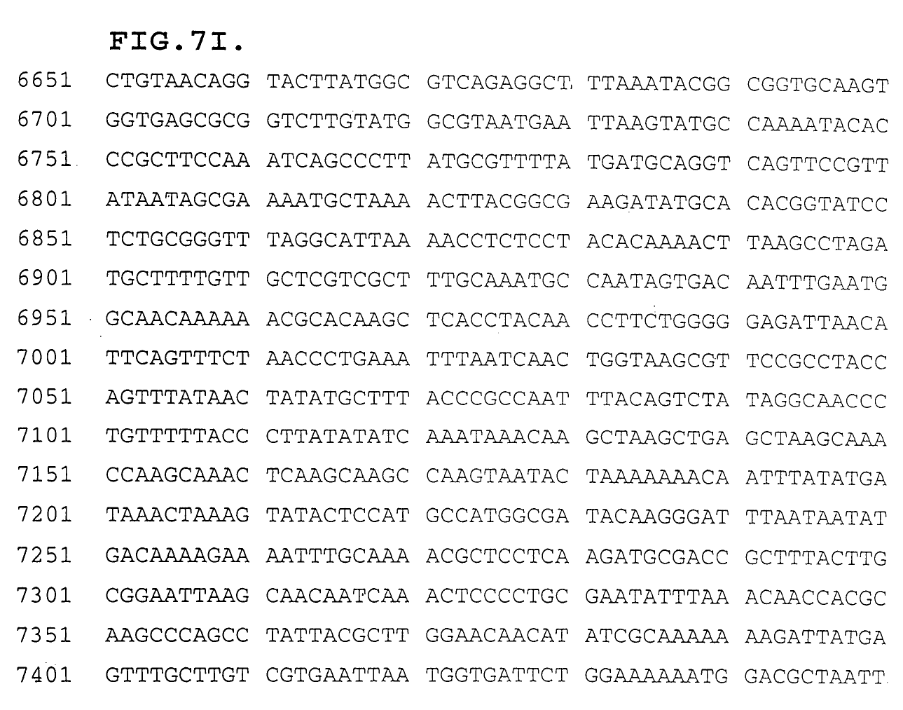

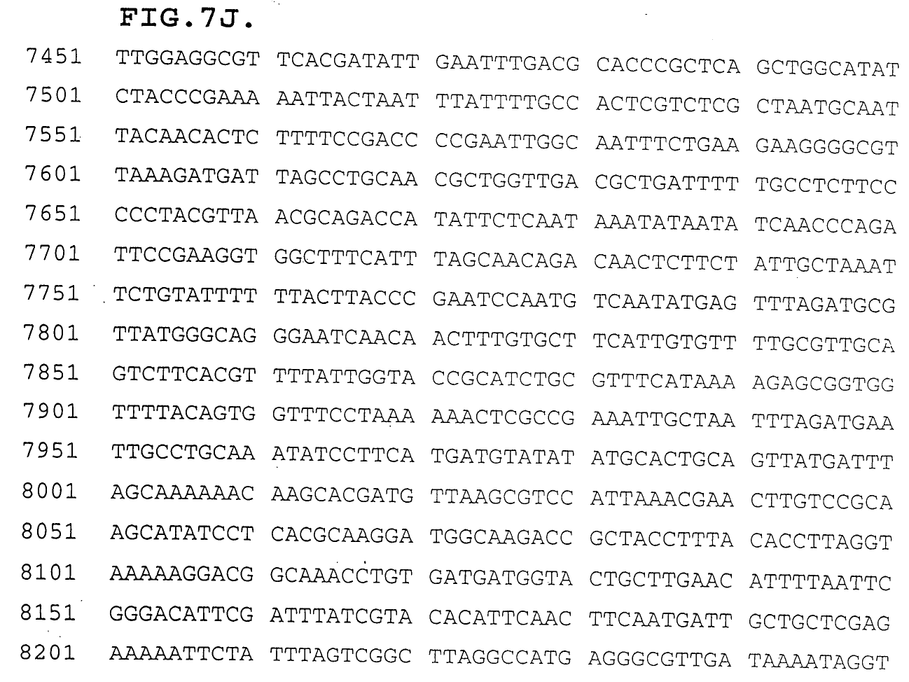

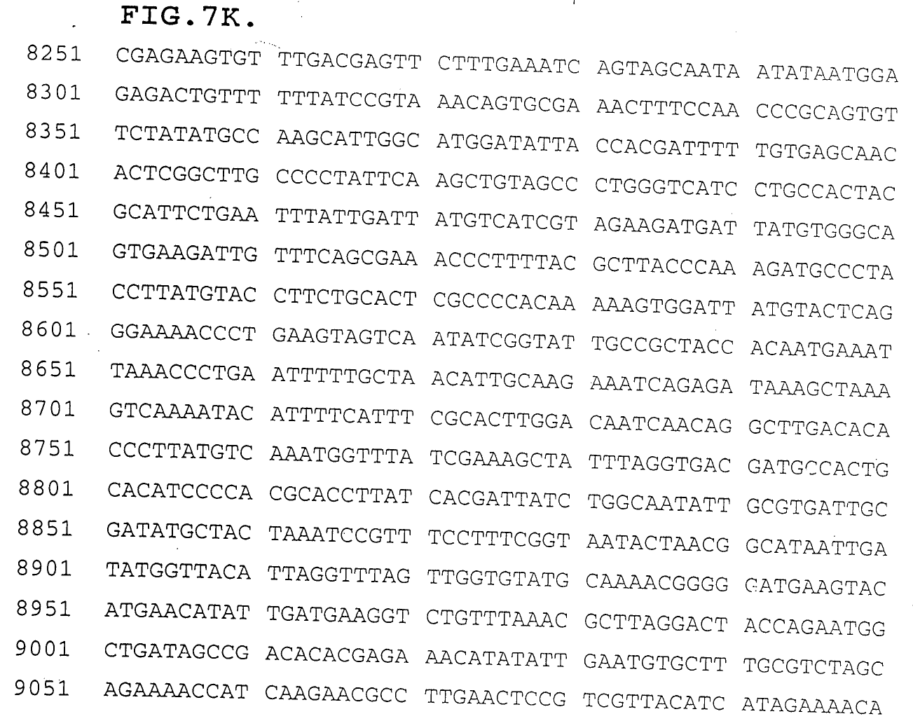

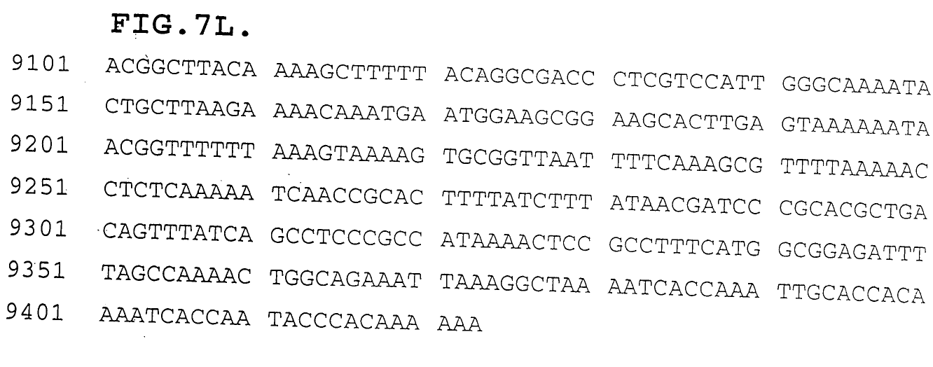

Subcloning studies with respect to the hmw1 and hmw2 genes indicated that correct processing of the HMW proteins required the products of additional downstream genes. It has been found that both the hmw1 and hmw2 genes are flanked by two additional downstream open reading frames (ORFs), designated b and c, respectively, (see FIGS. 6 and 7).

The b ORFs are 1635 bp in length, extending from nucleotides 5114 to 6748 in the case of hmw1 and nucleotides 5375 to 7009 in the case of hmw2, with their derived amino acid sequences being 99% identical. The derived amino acid sequences demonstrate similarity with the derived amino acid sequences of two genes which encode proteins required for secretion and activation of hemolysins of P. mirabilis and S. marcescens.

The c ORFs are 1950 bp in length, extending from nucleotides 7062 to 9011 in the case of hmw1 and nucleotides 7249 to 9198 in the case of hmw2, with their derived amino acid sequences 96% identical. The hmw1 c ORF is preceded by a series of 9 bp direct tandem repeats. In plasmid subclones, interruption of the hmw1 b or c ORF results in defective processing and secretion of the hmw1 structural gene product.

The two high molecular weight proteins HMW1 and HMW2 have been isolated and purified by the procedures described below in the Examples and shown to be protective against otitis media in chinchillas and to function as adhesins. These results indicate the potential for use of such high molecular proteins and structurally-related proteins of other non-typeable strains of Haemophilus influenzae as components in immunogenic compositions for protecting a susceptible host, such as a human infant, against disease caused by infection with non-typeable Haemophilus influenzae.

Since the proteins provided herein are good cross-reactive antigens and are present in the majority of non-typeable Haemophilus strains, it is evident that these HMW proteins may become integral constituents of a universal Haemophilus vaccine. Indeed, these proteins may be used not only as protective antigens against otitis, sinusitis and bronchitis caused by the non-typeable Haemophilus strains, but also may be used as carriers for the protective Hib polysaccharides in a conjugate vaccine against meningitis. The proteins also may be used as carriers for other antigens, haptens and polysaccharides from other organisms, so as to induce immunity to such antigens, haptens and polysaccharides.

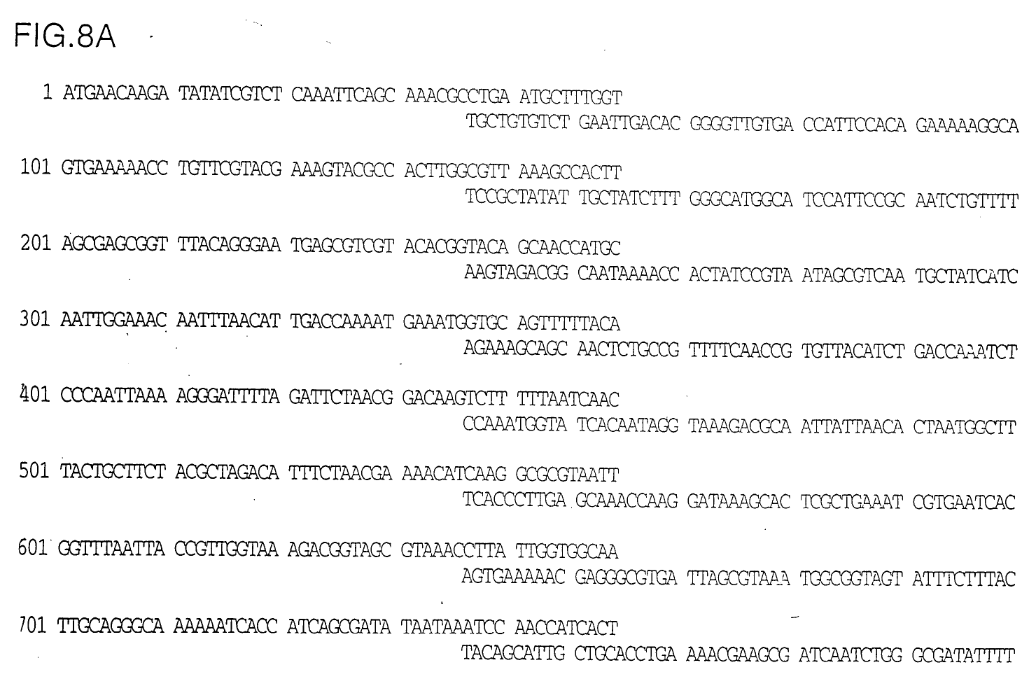

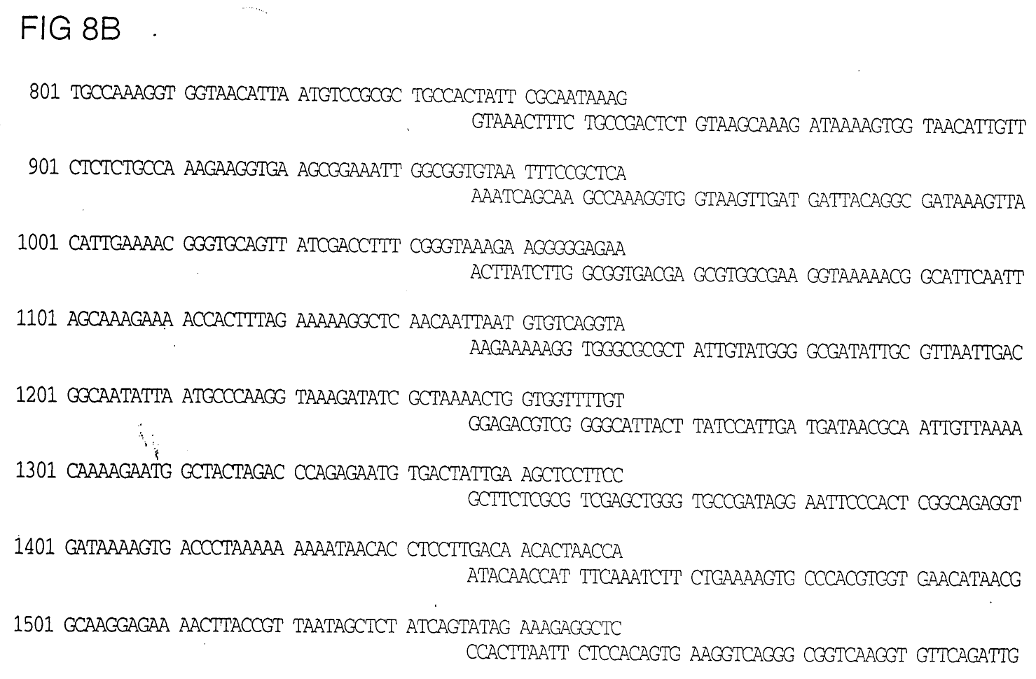

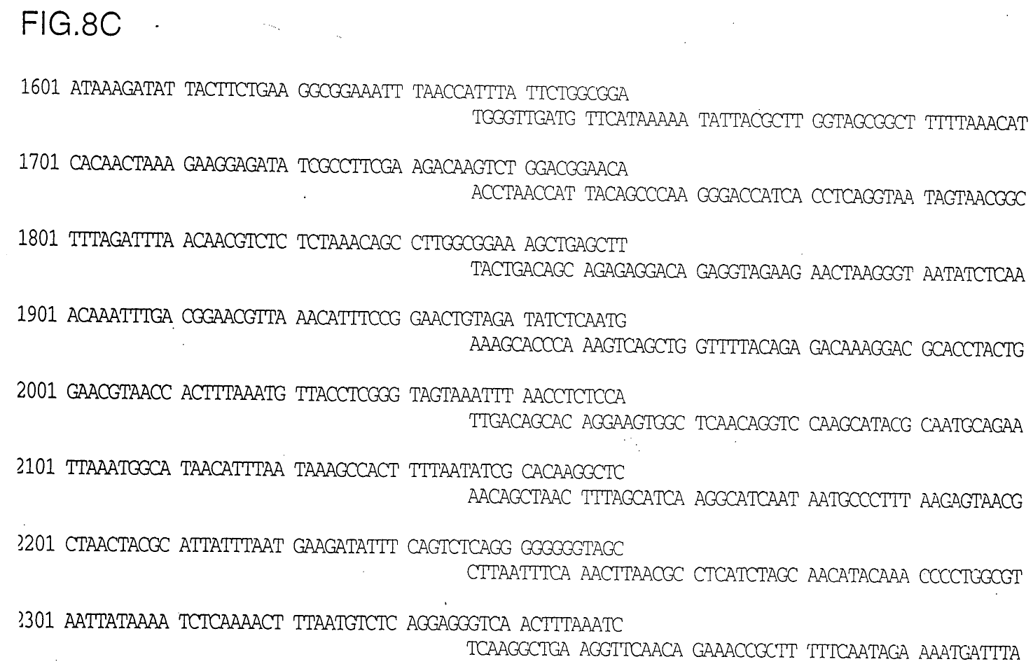

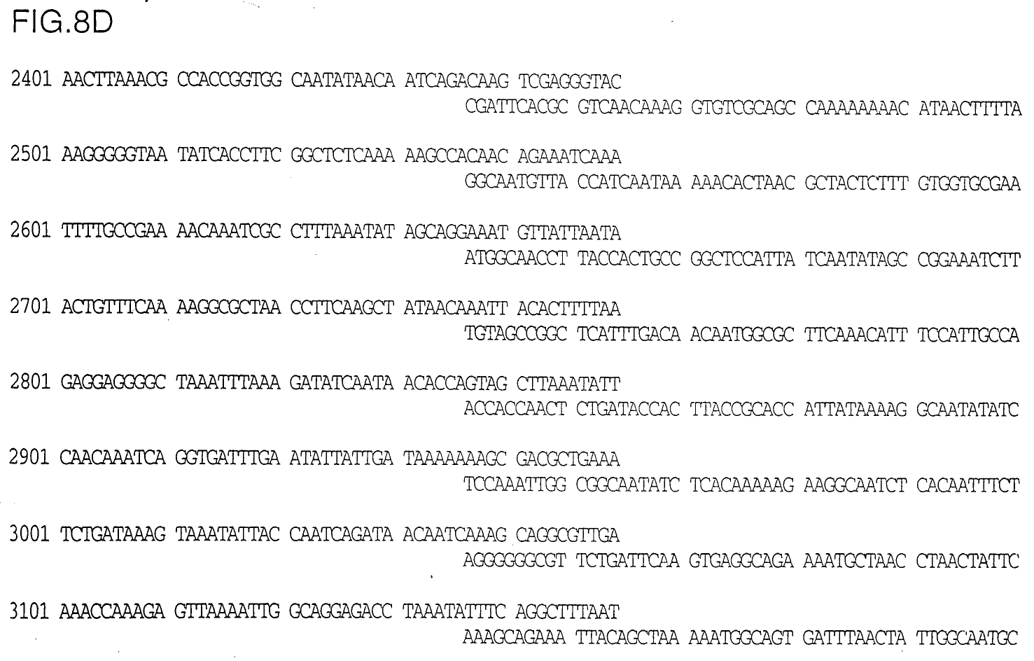

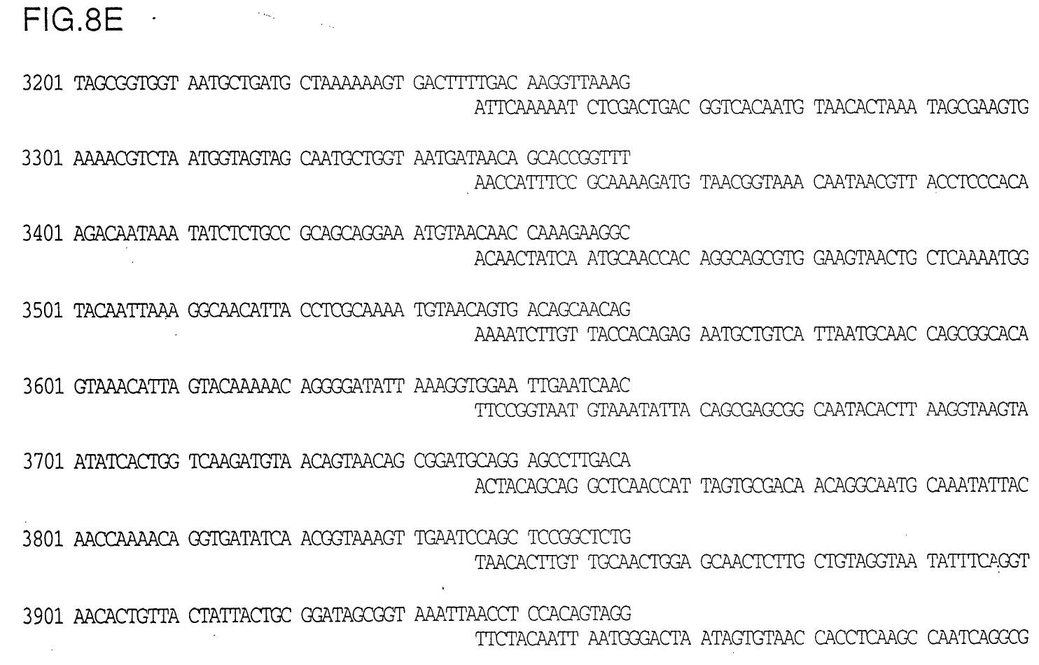

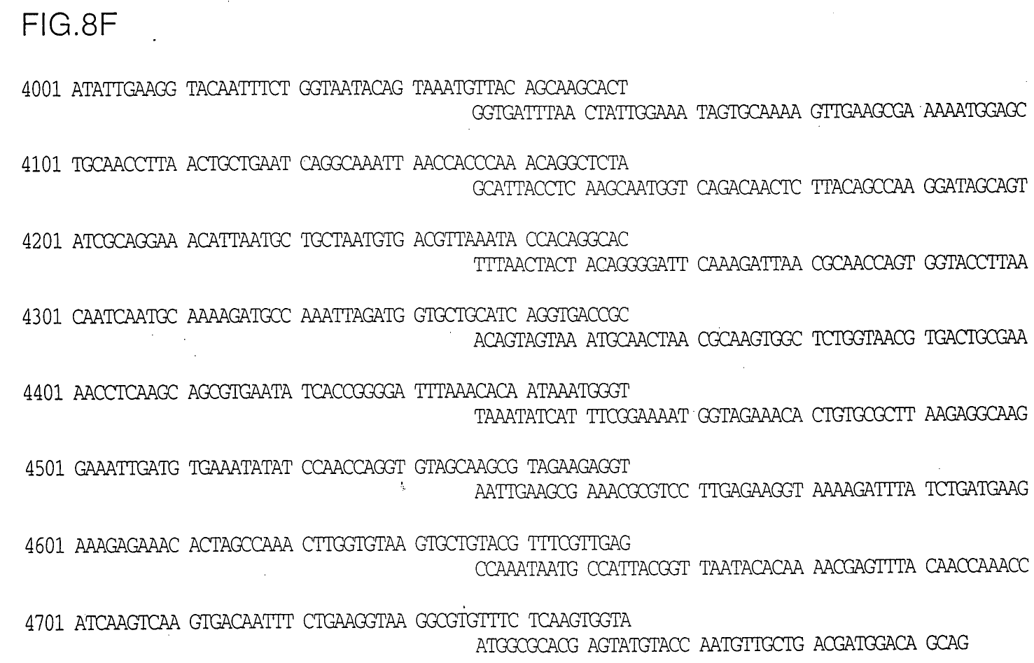

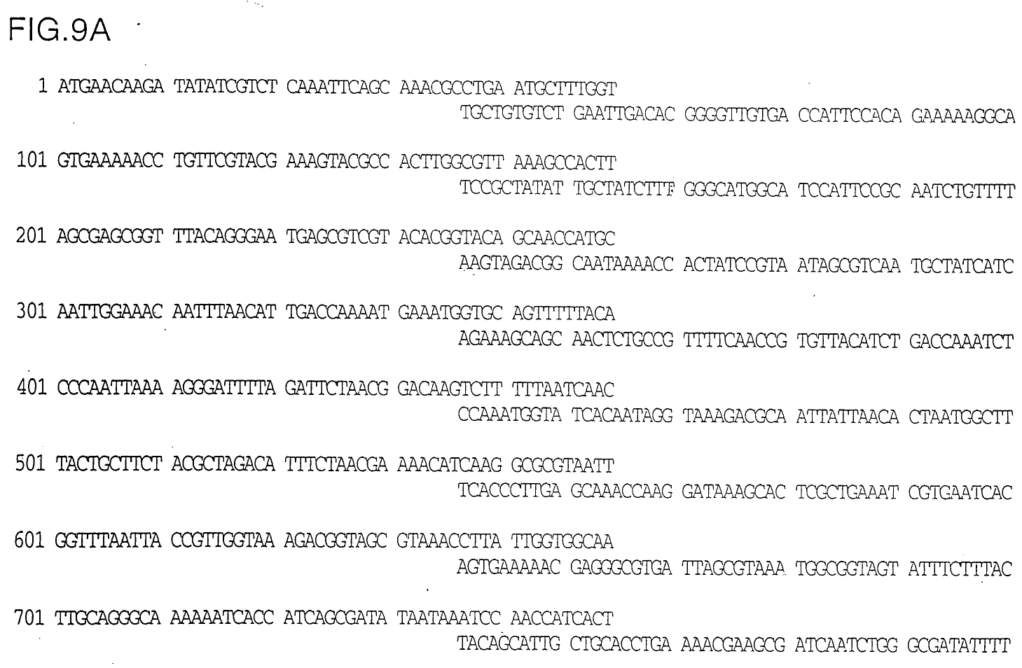

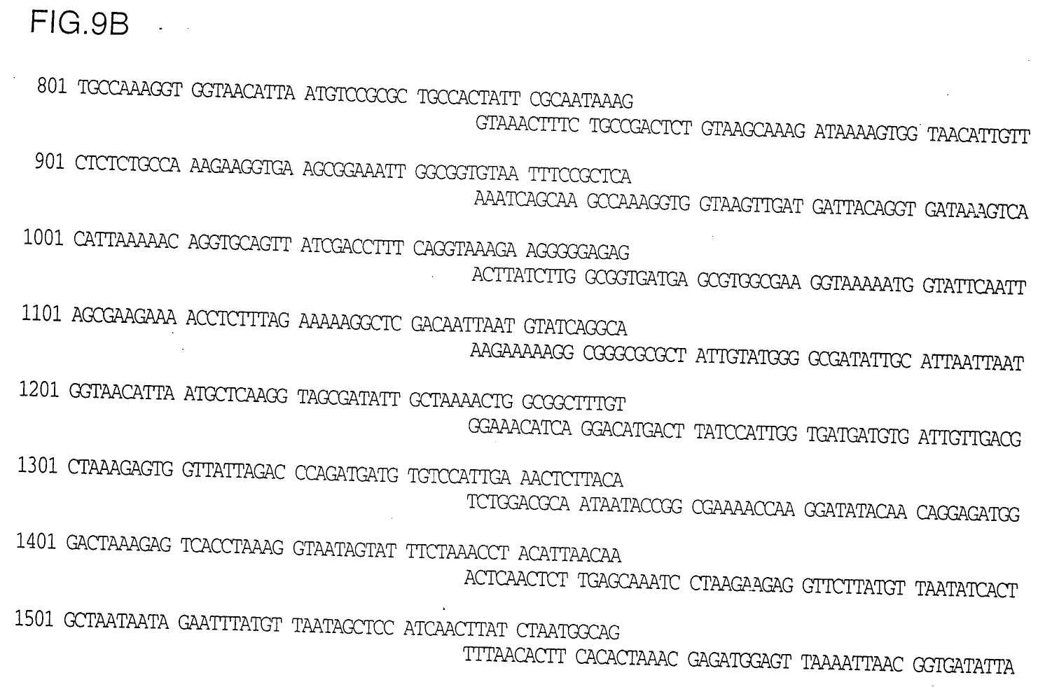

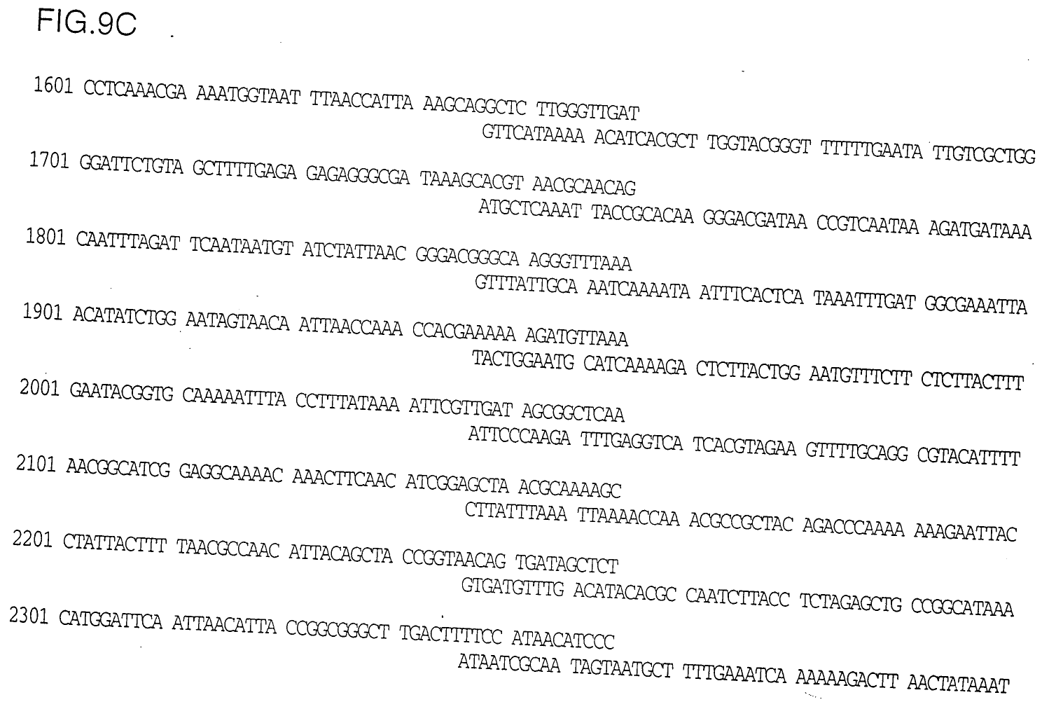

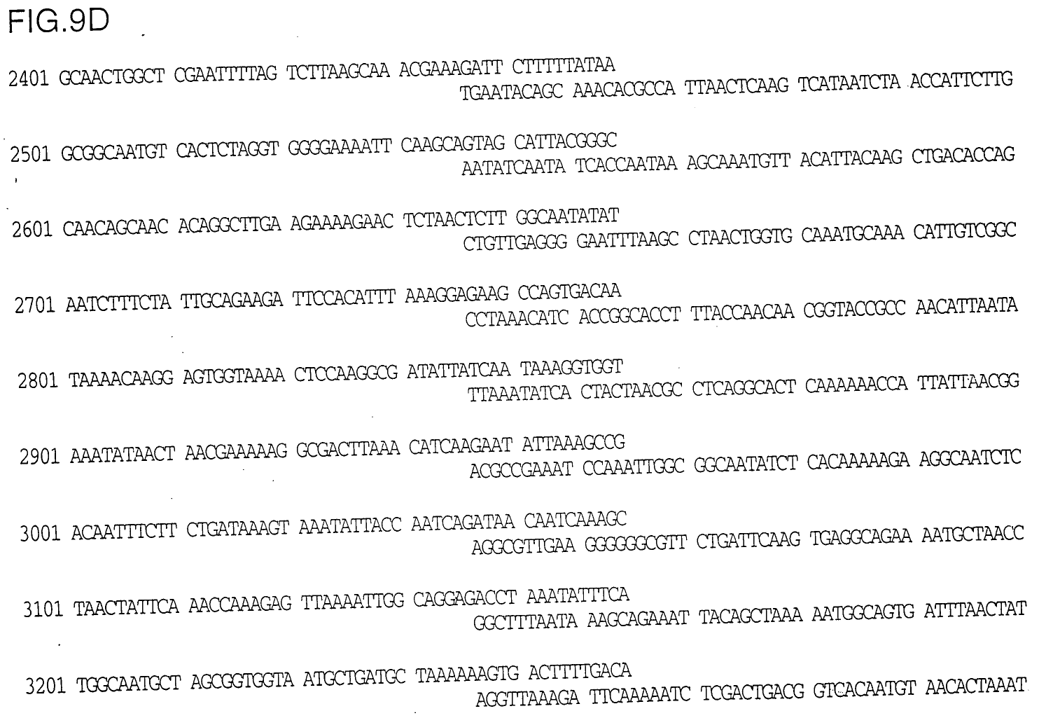

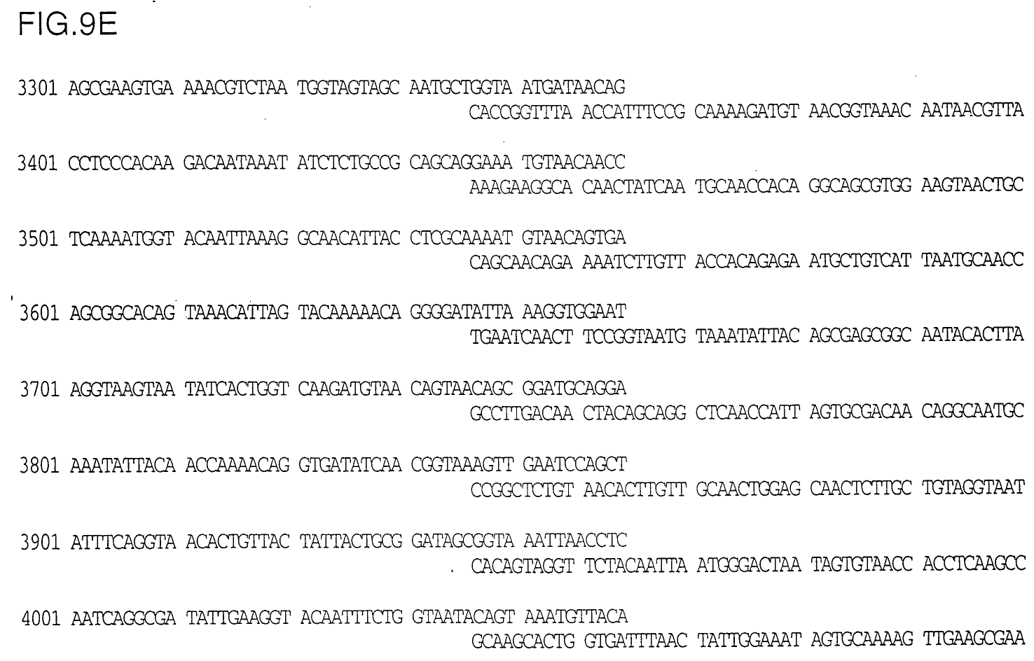

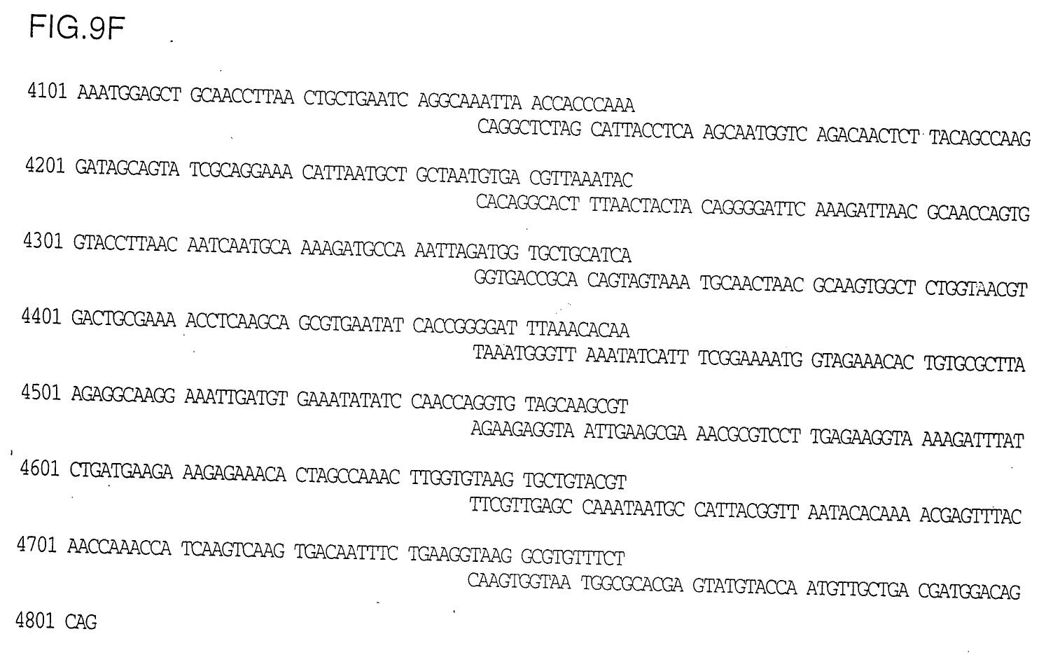

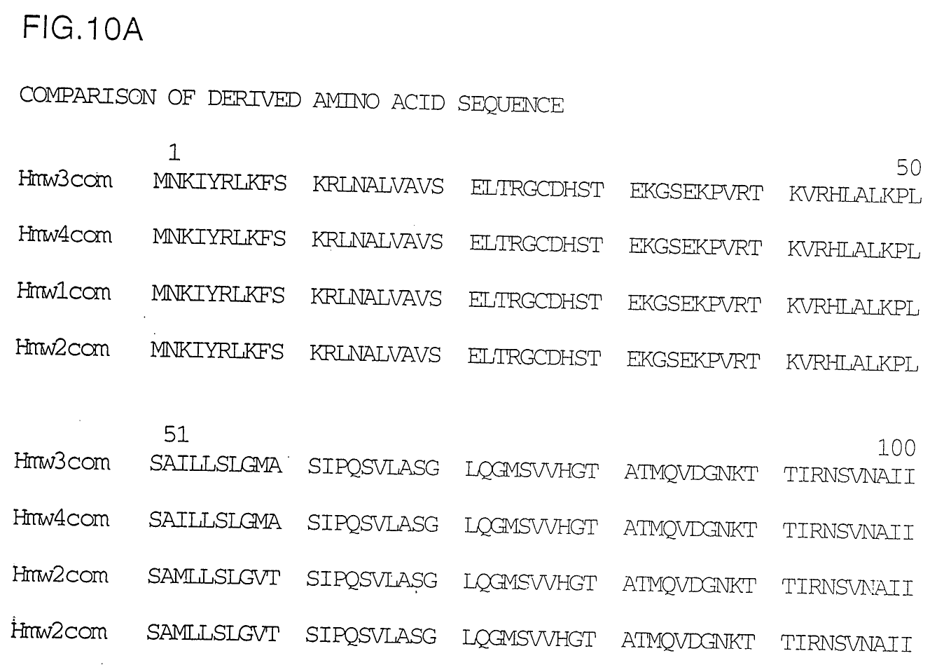

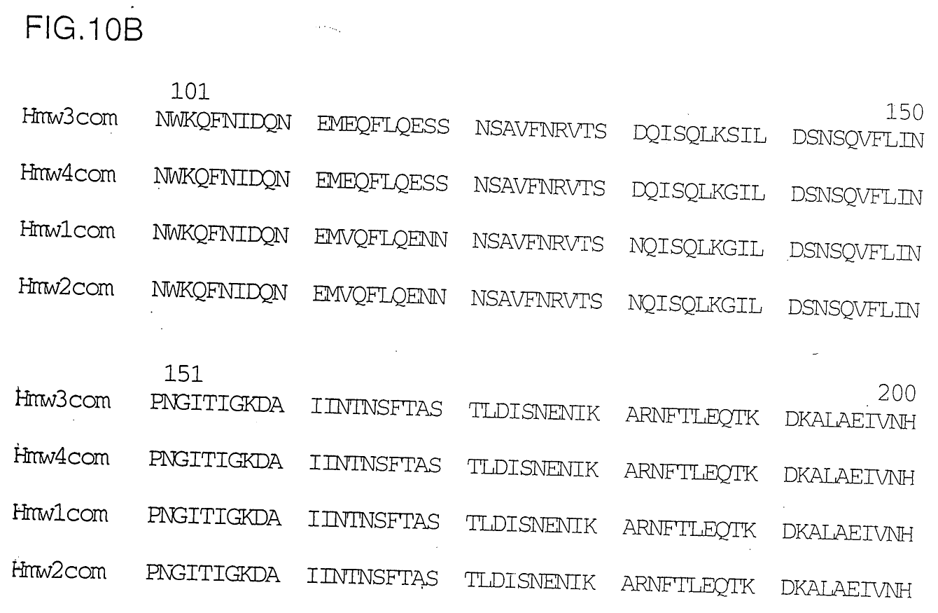

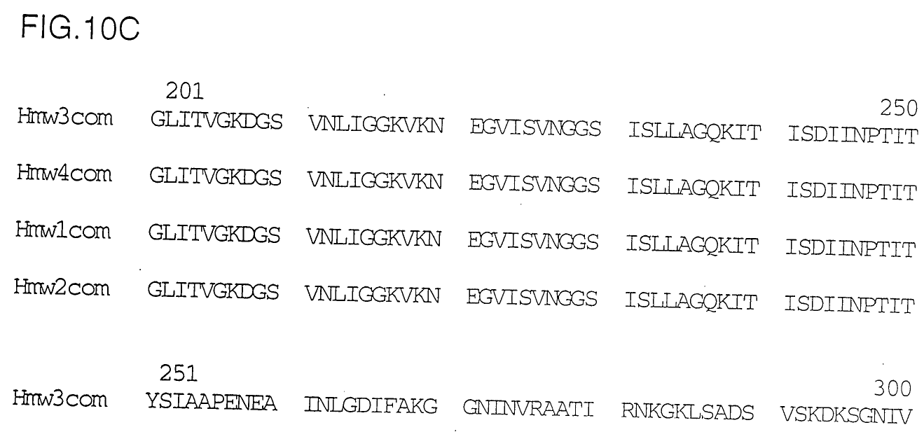

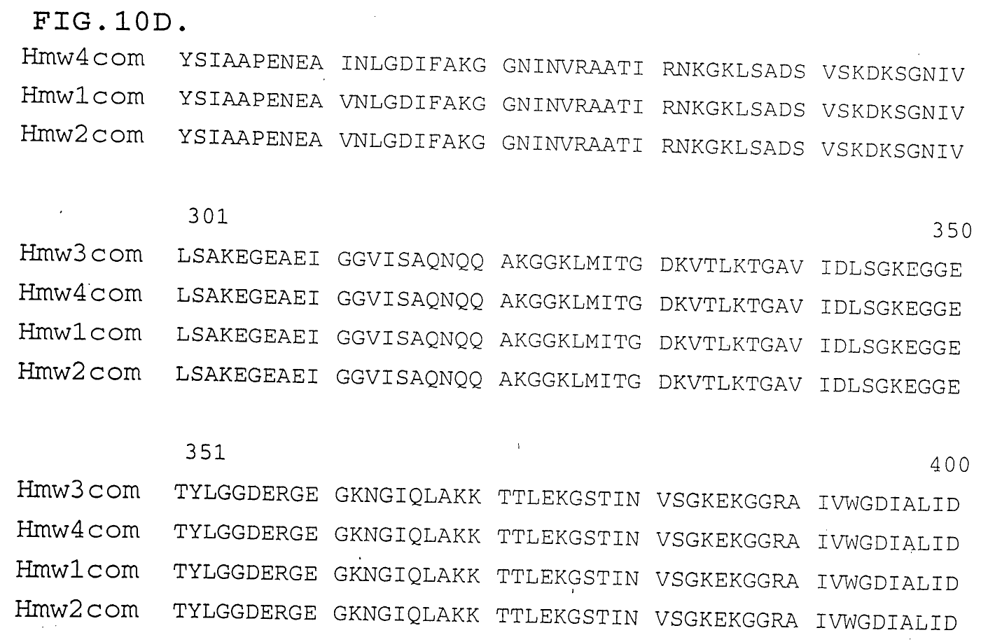

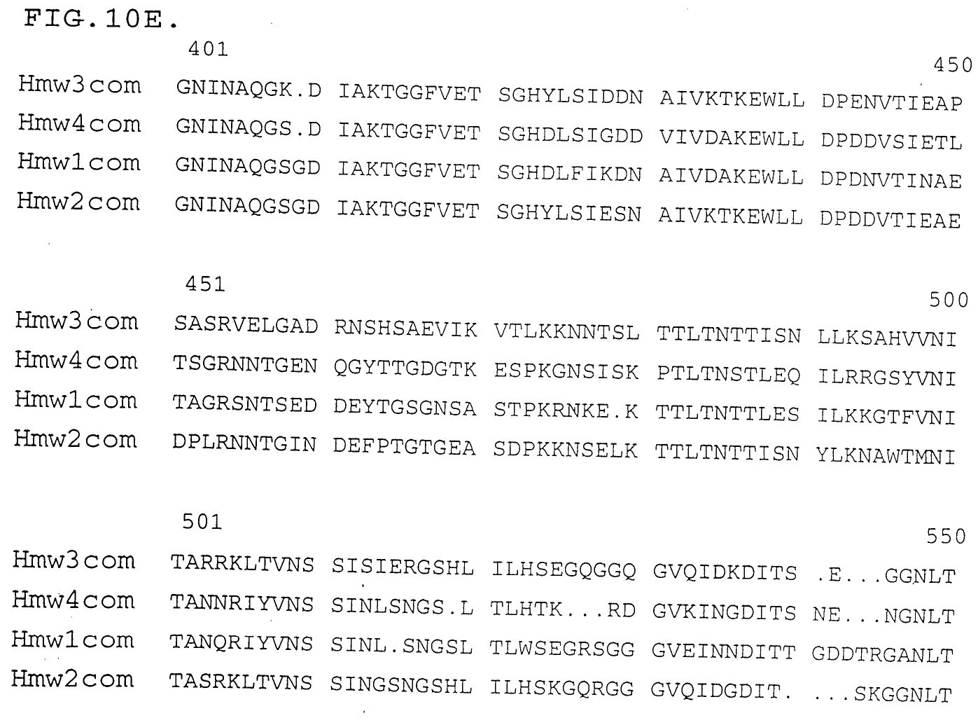

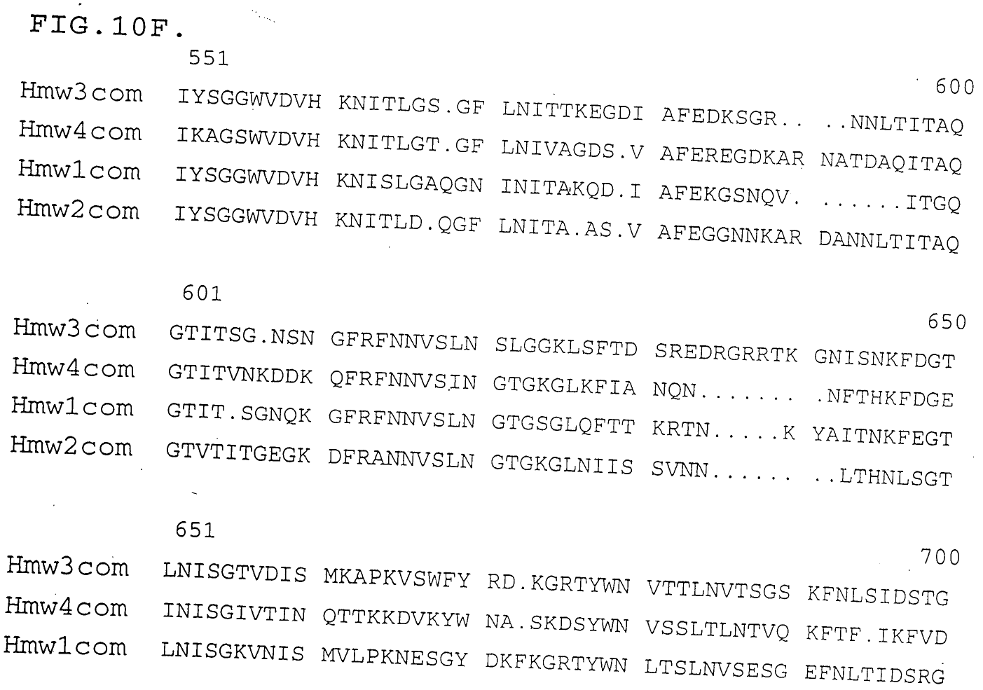

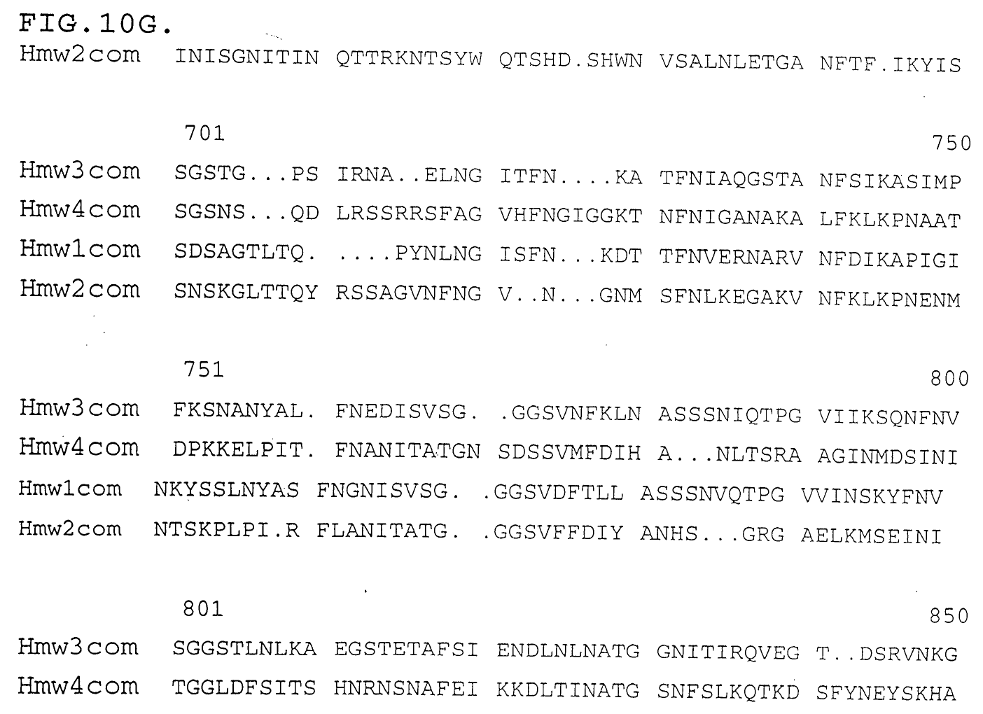

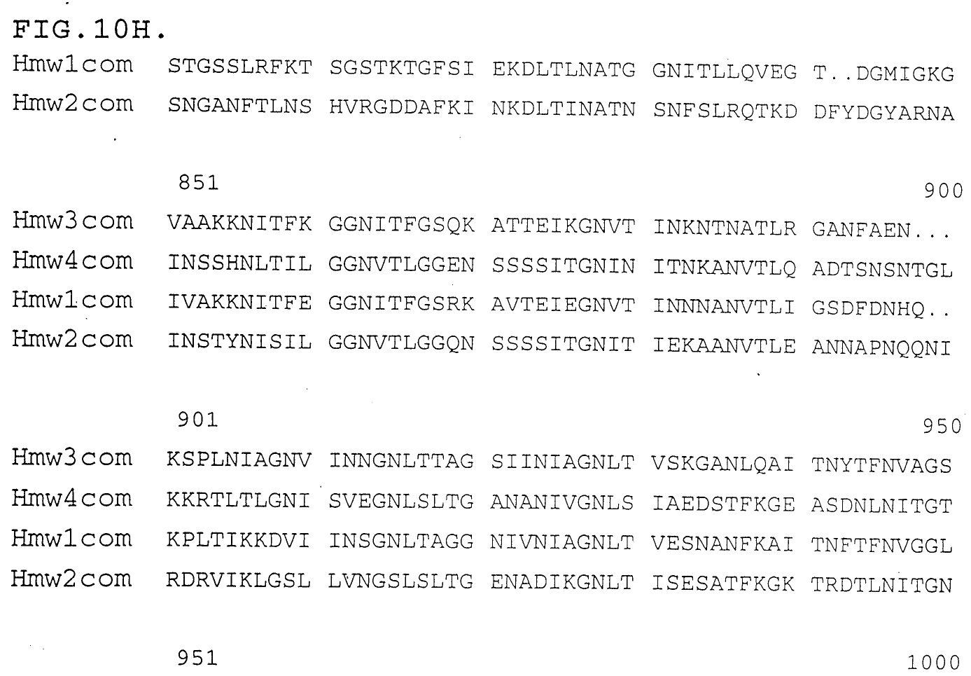

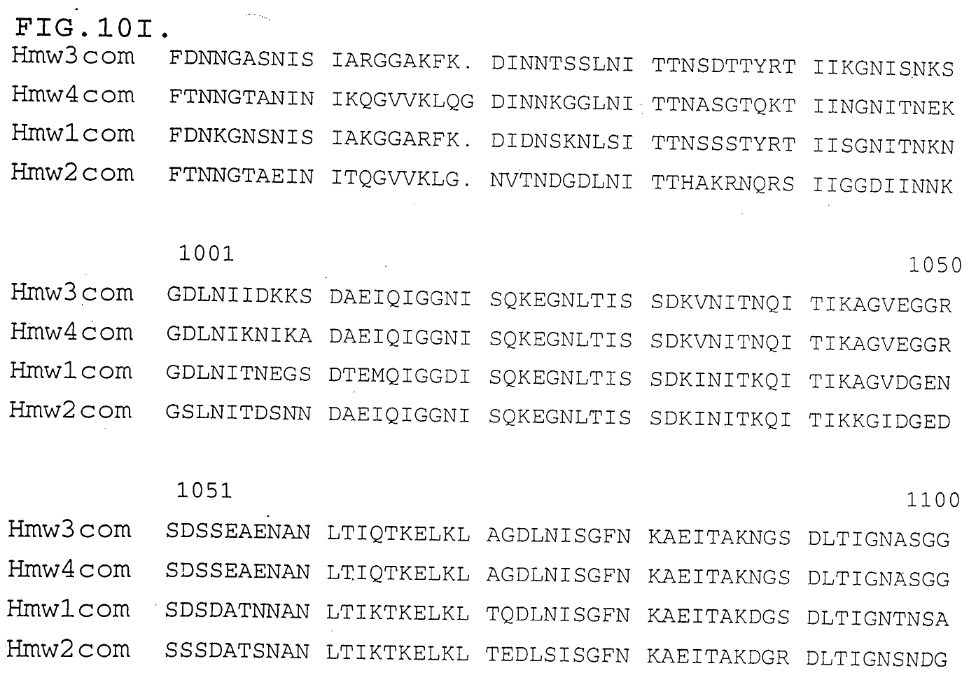

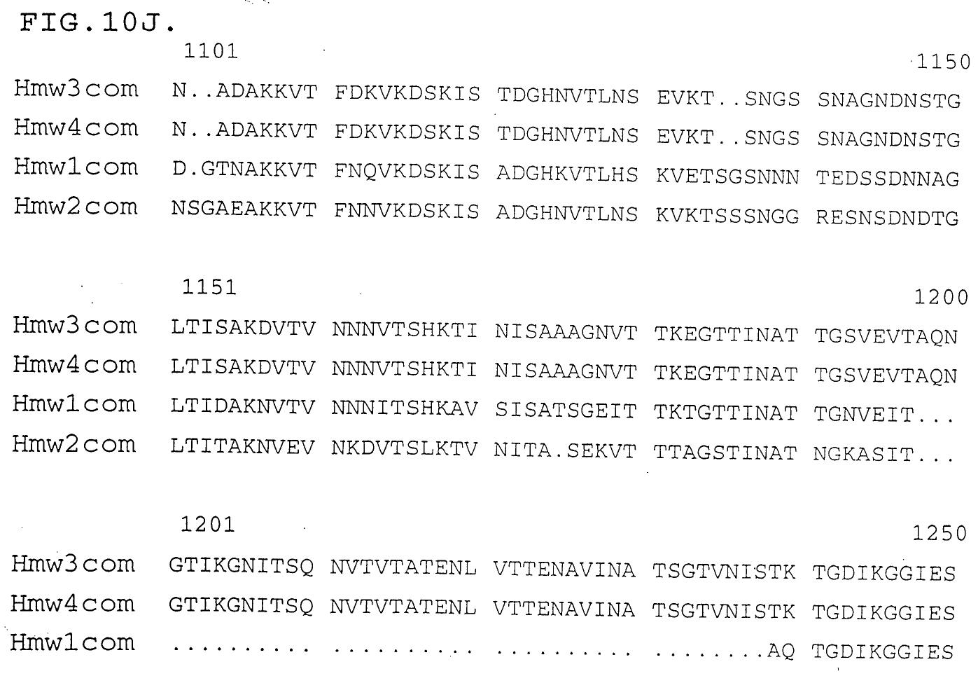

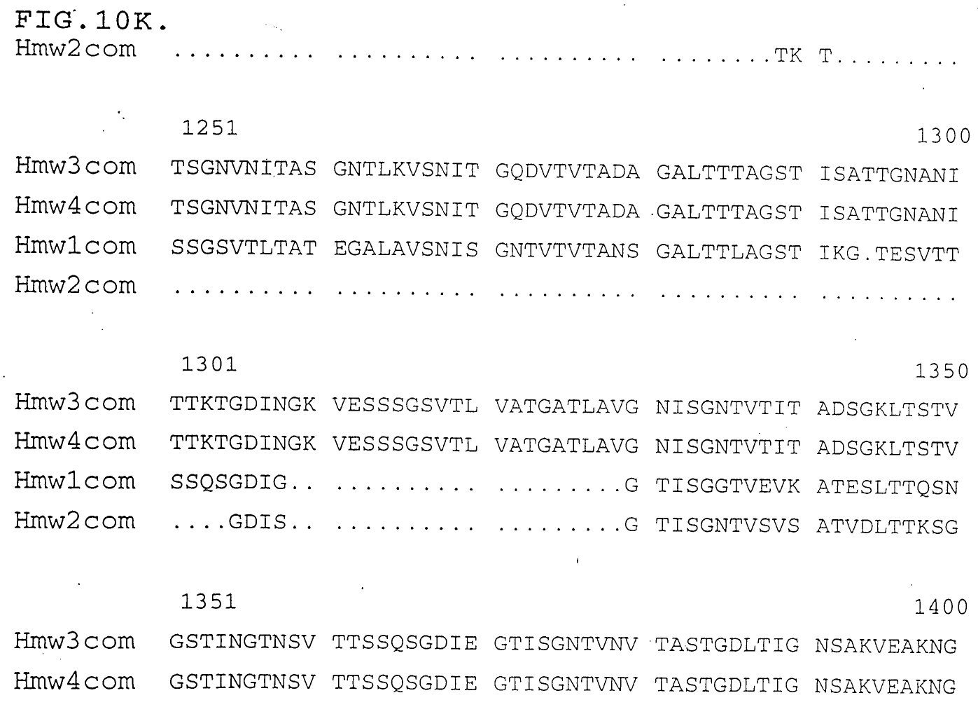

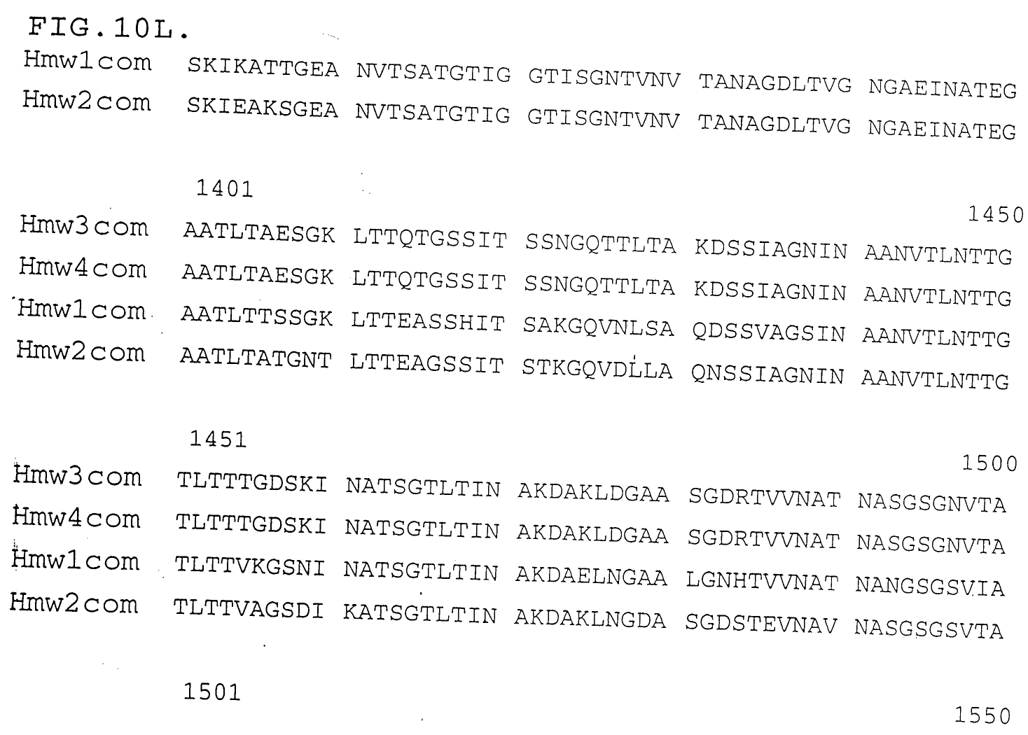

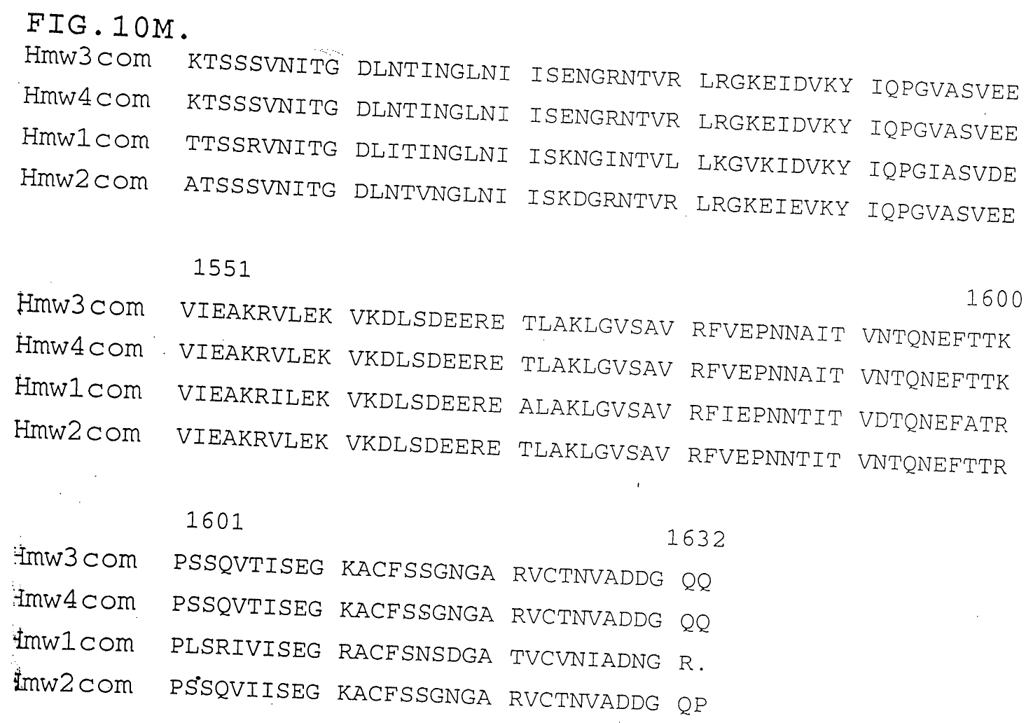

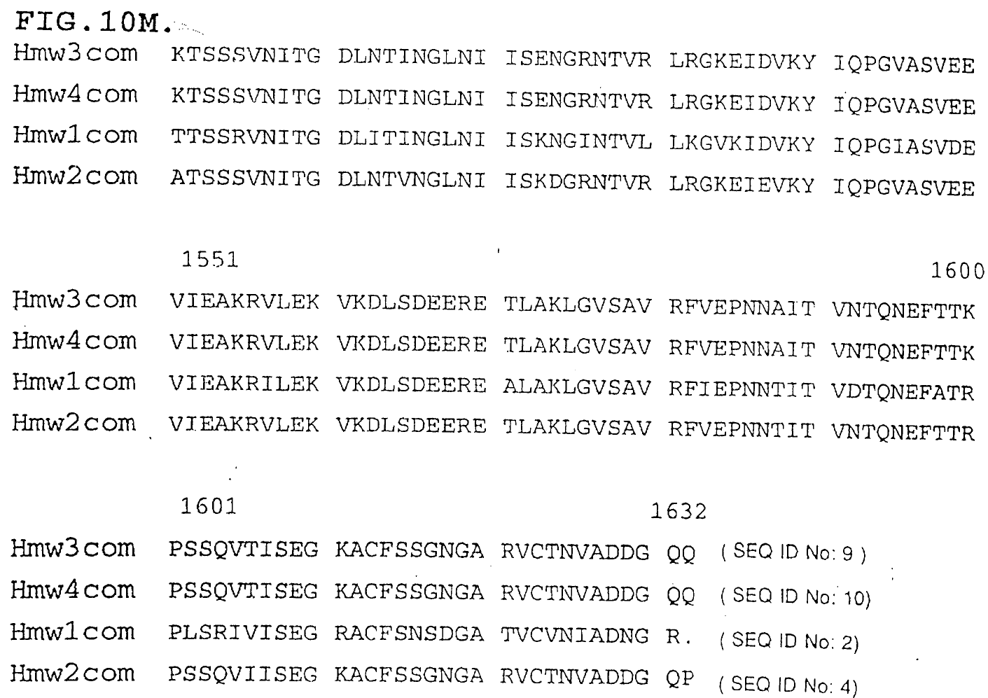

The nucleotide sequences encoding two high molecular weight proteins of a different non-typeable Haemophilus strain (designated HMW3 and HMW4), namely strain 5 have been elucidated, and are presented in FIGS. 8 and 9 (SEQ ID Nos: 7 and 8). HMW3 has an apparent molecular weight of 125 kDa while HMW4 has an apparent molecular weight of 123 kDa. These high molecular weight proteins are antigenically related to the HMW1 and HMW2 proteins and to FHA. FIG. 10 contains a multiple sequence comparison of the derived amino acid sequences for the four high molecular weight proteins identified herein (HMW1, SEQ ID No: 2; HMW2, SEQ ID No: 4; HMW3, SEQ ID No: 9; HMW4, SEQ ID No. 10). As may be seen from this comparison, stretches of identical amino acid sequence may be found throughout the length of the comparison, with HMW3 more closely resembling HMW1 and HMW4 more closely resembling HMW2. This information is highly suggestive of a considerable sequence homology between high molecular weight proteins from various non-typeable Haemophilus strains. This information is also suggestive that the HMW3 and HMW4 proteins will have the same immunological properties as the HMW1 and HMW2 proteins and that corresponding HMW proteins from other non-typeable Haemophilus strains will have the same immunological properties as the HMW1 and HMW2 proteins.

In addition, mutants of non-typeable H. influenzae strains that are deficient in expression of HMW1 or HMW2 or both have been constructed and examined for their capacity to adhere to cultured human epithelial cells. The hmw1 and hmw2 gene clusters have been expressed in E. coli and have been examined for in vitro adherence. The results of such experimentation, described below, demonstrate that both HMW1 and HMW2 mediate attachment and hence are adhesins and that this function is present even in the absence of other H. influenzae surface structures. The ability of a bacterial surface protein to function as an adhesin provides strong in vitro evidence for its potential role as a protective antigen. In view of the considerable sequence homology between the HMW3 and HMW4 proteins and the HMW1 and HMW2 proteins, these results indicate that HMW3 and HMW4 also are likely to function as adhesins and that other HMW proteins of other strains of non-typeable Haemophilus influenzae similarly are likely to function as adhesins. This expectation is borne out by the results described in the Examples below.

With the isolation and purification of the high molecular weight proteins, the inventor is able to determine the major protective epitopes of the proteins by conventional epitope mapping and synthesizing peptides corresponding to these determinants for incorporation into fully synthetic or recombinant vaccines. Accordingly, the invention also comprises a synthetic peptide having at least six and no more than 150 amino acids and having an amino acid sequence corresponding to at least one protective epitope of a high molecular weight protein of a non-typeable Haemophilus influenzae. Such peptides are of varying length that constitute portions of the high molecular weight proteins, that can be used to induce immunity, either directly or as part of a conjugate, against the respective organisms and thus constitute active components of immunogenic compositions for protection against the corresponding diseases.

In particular, the applicant has sought to identify regions of the high molecular weight proteins which are demonstrated experimentally to be surface-exposed B-cell epitopes and which are common to all or at least a large number of non-typeable strains of Haemophilus influenzae. The strategy which has been adopted by the inventor has been to:
(a) generate a panel of monoclonal antibodies reactive with the high molecular weight proteins;
(b) screen those monoclonal antibodies for reactivity with surface epitopes of intact bacteria using immunoelectron microscopy or other suitable screening technique;
(c) map the epitopes recognized by the monoclonal antibody by determining the reactivity of the monoclonals with a panel of recombinant fusion proteins; and
(d) determining the reactivity of the monoclonal antibodies with heterologous non-typable Haemophilus influenzae strains using standard Western blot assays.
   Using this approach, the inventor has identified one monoclonal antibody, designated AD6 (ATCC ______), which recognized a surface-exposed B-cell epitope common to all non-typeable H. influenzae which express the HMW1 and HMW2 proteins. The epitope recognized by this antibody was mapped to a 75 amino acid sequence at the carboxy termini of both HMW1 and HMW2 proteins. The ability to identify shared surface-exposed epitopes on the high molecular weight adhesion proteins suggests that it would be possible to develop recombinant or synthetic peptide based vaccines which would be protective against disease caused by the majority of non-typeable Haemophilus influenzae.

The present invention also provides any variant or fragment of the proteins that retains the potential immunological ability to protect against disease caused by non-typeable Haemophilus strains. The variants may be constructed by partial deletions or mutations of the genes and expression of the resulting modified genes to give the protein variants.

It is clearly apparent to one skilled in the art, that the various embodiments of the present invention have many applications in the fields of vaccination, diagnosis, treatment of bacterial infections and the generation of immunological reagents. A further non-limiting discussion of such uses is further presented below.

1. Vaccine Preparation and Use

Immunogenic compositions, suitable to be used as vaccines, may be prepared from the high molecular weight proteins of Haemophilus influenzae, as well as analogs and fragments thereof, and synthetic peptides containing epitopes of the protein, as disclosed herein. The immunogenic composition elicits an immune response which produces antibodies, including anti-high molecular weight protein antibodies and antibodies that are opsonizing or bactericidal.

Immunogenic compositions, including vaccines, may be prepared as injectables, as liquid solutions or emulsions. The active component may be mixed with pharmaceutically acceptable excipients which are compatible therewith. Such excipients may include, water, saline, dextrose, glycerol, ethanol, and combinations thereof. The immunogenic compositions and vaccines may further contain auxiliary substances, such as wetting or emulsifying agents, pH buffering agents, or adjuvants to enhance the effectiveness thereof. Immunogenic compositions and vaccines may be administered parenterally, by injection subcutaneously or intramuscularly. Alternatively, the immunogenic compositions formed according to the present invention, may be formulated and delivered in a manner to evoke an immune response at mucosal surfaces. Thus, the immunogenic composition may be administered to mucosal surfaces by, for example, the nasal or oral (intragastric) routes. Alternatively, other modes of administration including suppositories and oral formulations may be desirable. For suppositories, binders and carriers may include, for example, polyalkalene glycols or triglycerides. Oral formulations may include normally employed incipients such as, for example, pharmaceutical grades of saccharine, cellulose and magnesium carbonate. These compositions can take the form of solutions, suspensions, tablets, pills, capsules, sustained release formulations or powders and contain about 1 to 95% of the active component. The immunogenic preparations and vaccines are administered in a manner compatible with the dosage formulation, and in such amount as will be therapeutically effective, protective and immunogenic. The quantity to be administered depends on the subject to be treated, including, for example, the capacity of the individual's immune system to synthesize antibodies, and if needed, to produce a cell-mediated immune response. Precise amounts of active ingredient required to be administered depend on the judgment of the practitioner. However, suitable dosage ranges are readily determinable by one skilled in the art and may be of the order of micrograms of the HMW proteins. Suitable regimes for initial administration and booster doses are also variable, but may include an initial administration followed by subsequent administrations. The dosage may also depend on the route of administration and will vary according to the size of the host.

The concentration of the active component in an immunogenic composition according to the invention is in general about 1 to 95%. A vaccine which contains antigenic material of only one pathogen is a monovalent vaccine. Vaccines which contain antigenic material of several pathogens are combined vaccines and also belong to the present invention. Such combined vaccines contain, for example, material from various pathogens or from various strains of the same pathogen, or from combinations of various pathogens.

Immunogenicity can be significantly improved if the antigens are co-administered with adjuvants, commonly used as 0.05 to 0.1 percent solution in phosphate-buffered saline. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses.

Immunostimulatory agents or adjuvants have been used for many years to improve the host immune responses to, for example, vaccines. Intrinsic adjuvants, such as lipopolysaccharides, normally are the components of the killed or attenuated bacteria used as vaccines. Extrinsic adjuvants are immunomodulators which are typically non-covalently linked to antigens and are formulated to enhance the host immune responses. Thus, adjuvants have been identified that enhance the immune response to antigens delivered parenterally. Some of these adjuvants are toxic, however, and can cause undesirable side-effects, making them unsuitable for use in humans and many animals. Indeed, only aluminum hydroxide and aluminum phosphate (collectively commonly referred to as alum) are routinely used as adjuvants in human and veterinary vaccines. The efficacy of alum in increasing antibody responses to diphtheria and tetanus toxoids is well established and a HBsAg vaccine has been adjuvanted with alum. While the usefulness of alum is well established for some applications, it has limitations. For example, alum is ineffective for influenza vaccination and inconsistently elicits a cell mediated immune response. The antibodies elicited by alum-adjuvanted antigens are mainly of the IgG1 isotype in the mouse, which may not be optimal for protection by some vaccinal agents.

A wide range of extrinsic adjuvants can provoke potent immune responses to antigens. These include saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, and liposomes.

To efficiently induce humoral immune responses (HIR) and cell-mediated immunity (CMI), immunogens are often emulsified in adjuvants. Many adjuvants are toxic, inducing granulomas, acute and chronic inflammations (Freund's complete adjuvant, FCA), cytolysis (saponins and Pluronic polymers) and pyrogenicity, arthritis and anterior uveitis (LPS and MDP). Although FCA is an excellent adjuvant and widely used in research, it is not licensed for use in human or veterinary vaccines because of its toxicity.

Desirable characteristics of ideal adjuvants include:
(1) lack of toxicity;
(2) ability to stimulate a long-lasting immune response;
(3) simplicity of manufacture and stability in long-term storage;
(4) ability to elicit both CMI and HIR to antigens administered by various routes, if required;
(5) synergy with other adjuvants;
(6) capability of selectively interacting with populations of antigen presenting cells (APC);
(7) ability to specifically elicit appropriate T_{H}1 or T_{H}2 cell-specific immune responses; and
(8) ability to selectively increase appropriate antibody isotype levels (for example, IgA) against antigens.

U.S. Pat. No. 4,855,283 granted to Lockhoff et al on Aug. 8, 1989 which is incorporated herein by reference thereto teaches glycolipid analogues including N-glycosylamides, N-glycosylureas and N-glycosylcarbamates, each of which is substituted in the sugar residue by an amino acid, as immuno-modulators or adjuvants. Thus, Lockhoff et al. (U.S. Pat. No. 4,855,283 and ref. 29) reported that N-glycolipid analogs displaying structural similarities to the naturally-occurring glycolipids, such as glycosphingolipids and glycoglycerolipids, are capable of eliciting strong immune responses in both herpes simplex virus vaccine and pseudorabies virus vaccine. Some glycolipids have been synthesized from long chain-alkylamines and fatty acids that are linked directly with the sugars through the anomeric carbon atom, to mimic the functions of the naturally occurring lipid residues.

U.S. Pat. No. 4,258,029 granted to Moloney, incorporated herein by reference thereto, teaches that octadecyl tyrosine hydrochloride (OTH) functioned as an adjuvant when complexed with tetanus toxoid and formalin inactivated type I, II and III poliomyelitis virus vaccine. Also, Nixon-George et al. (ref. 30), reported that octadecyl esters of aromatic amino acids complexed with a recombinant hepatitis B surface antigen, enhanced the host immune responses against hepatitis B virus. Lipidation of synthetic peptides has also been used to increase their immunogenicity. Thus, Wiesmuller 1989, describes a peptide with a sequence homologous to a foot-and-mouth disease viral protein coupled to an adjuvant tripalmityl-s-glyceryl-cysteinylserylserine, being a synthetic analogue of the N-terminal part of the lipoprotein from Gram negative bacteria. Furthermore, Deres et al. 1989, reported in vivo priming of virus-specific cytotoxic T lymphocytes with synthetic lipopeptide vaccine which comprised of modified synthetic peptides derived from influenza virus nucleoprotein by linkage to a lipopeptide, N-palmityl-s-[2,3-bis(palmitylxy)-(2RS)-propyl-[R]-cysteine (TPC).

2. Immunoassays

The high molecular weight protein of Haemophilus influenzae of the present invention is useful as an immunogen for the generation of anti-protein antibodies, as an antigen in immunoassays including enzyme-linked immunosorbent assays (ELISA), RIAs and other non-enzyme linked antibody binding assays or procedures known in the art for the detection of antibodies. In ELISA assays, the protein is immobilized onto a selected surface, for example, a surface capable of binding proteins, such as the wells of a polystyrene microtiter plate. After washing to remove incompletely adsorbed protein, a nonspecific protein, such as a solution of bovine serum albumin (BSA) that is known to be antigenically neutral with regard to the test sample, may be bound to the selected surface. This allows for blocking of nonspecific adsorption sites on the immobilizing surface and thus reduces the background caused by nonspecific bindings of antisera onto the surface. The immobilizing surface is then contacted with a sample, such as clinical or biological materials, to be tested in a manner conducive to immune complex (antigen/antibody) formation. This may include diluting the sample with diluents, such as solutions of BSA, bovine gamma globulin (BGG) and/or phosphate buffered-saline (PBS)/Tween. The sample is then allowed to incubate for from about 2 to 4 hours, at temperatures such as of the order of about 25° to 37° C. Following incubation, the sample-contacted surface is washed to remove non-immunocomplexed material. The washing procedure may include washing with a solution, such as PBS/Tween or a borate buffer. Following formation of specific immunocomplexes between the test sample and the bound protein, and subsequent washing, the occurrence, and even amount, of immunocomplex formation may be determined by subjecting the immunocomplex to a second antibody having specificity for the first antibody. If the test sample is of human origin, the second antibody is an antibody having specificity for human immunoglobulins and in general IgG. To provide detecting means, the second antibody may have an associated activity such as an enzymatic activity that will generate, for example, a colour development upon incubating with an appropriate chromogenic substrate. Quantification may then be achieved by measuring the degree of colour generation using, for example, a visible spectra spectrophotometer.

3. Use of Sequences as Hybridization Probes

The nucleotide sequences of the present invention, comprising the sequences of the genes encoding the high molecular weight proteins of specific strains of non-typeable Haemophilus influenzae, now allow for the identification and cloning of the genes from any species of non-typeable Haemophilus and other strains of non-typeable Haemophilus influenzae.

The nucleotide sequences comprising the sequences of the genes of the present invention are useful for their ability to selectively form duplex molecules with complementary stretches of other genes of high molecular weight proteins of non-typeable Haemophilus. Depending on the application, a variety of hybridization conditions may be employed to achieve varying degrees of selectivity of the probe toward the other genes. For a high degree of selectivity, relatively stringent conditions are used to form the duplexes, such as low salt and/or high temperature conditions, such as provided by 0.02 M to 0.15 M NaCl at temperatures of between about 50° C. to 70° C. For some applications, less stringent hybridization conditions are required such as 0.15 M to 0.9 M salt, at temperatures ranging from between about 20° C. to 55° C. Hybridization conditions can also be rendered more stringent by the addition of increasing amounts of formamide, to destabilize the hybrid duplex. Thus, particular hybridization conditions can be readily manipulated, and will generally be a method of choice depending on the desired results. In general, convenient hybridization temperatures in the presence of 50% formamide are: 42° C. for a probe which is 95 to 100% homologous to the target fragment, 37° C. for 90 to 95% homology and 32° C. for 85 to 90% homology.

In a clinical diagnostic embodiment, the nucleic acid sequences of the genes of the present invention may be used in combination with an appropriate means, such as a label, for determining hybridization. A wide variety of appropriate indicator means are known in the art, including radioactive, enzymatic or other ligands, such as avidin/biotin, which are capable of providing a detectable signal. In some diagnostic embodiments, an enzyme tag such as urease, alkaline phosphatase or peroxidase, instead of a radioactive tag may be used. In the case of enzyme tags, colorimetric indicator substrates are known which can be employed to provide a means visible to the human eye or spectrophotometrically, to identify specific hybridization with samples containing gene sequences encoding high molecular weight proteins of non-typeable Haemophilus.

The nucleic acid sequences of genes of the present invention are useful as hybridization probes in solution hybridizations and in embodiments employing solid-phase procedures. In embodiments involving solid-phase procedures, the test DNA (or RNA) from samples, such as clinical samples, including exudates, body fluids (e.g., serum, amniotic fluid, middle ear effusion, sputum, bronchoalveolar lavage fluid) or even tissues, is adsorbed or otherwise affixed to a selected matrix or surface. The fixed, single-stranded nucleic acid is then subjected to specific hybridization with selected probes comprising the nucleic acid sequences of the genes or fragments thereof of the present invention under desired conditions. The selected conditions will depend on the particular circumstances based on the particular criteria required depending on, for example, the G+C contents, type of target nucleic acid, source of nucleic acid, size of hybridization probe etc. Following washing of the hybridization surface so as to remove non-specifically bound probe molecules, specific hybridization is detected, or even quantified, by means of the label. As with the selection of peptides, it is preferred to select nucleic acid sequence portions which are conserved among species of non-typeable Haemophilus. The selected probe may be at least about 18 bp and may be in the range of about 30 bp to about 90 bp long.

4. Expression of the High Molecular Weight Protein Genes

Plasmid vectors containing replicon and control sequences which are derived from species compatible with the host cell may be used for the expression of the genes encoding high molecular weight proteins of non-typeable Haemophilus in expression systems. The vector ordinarily carries a replication site, as well as marking sequences which are capable of providing phenotypic selection in transformed cells. For example, E. coli may be transformed using pBR322 which contains genes for ampicillin and tetracycline resistance and thus provides easy means for identifying transformed cells. The pBR322 plasmid, or other microbial plasmid or phage must also contain, or be modified to contain, promoters which can be used by the host cell for expression of its own proteins.

In addition, phage vectors containing replicon and control sequences that are compatible with the host can be used as a transforming vector in connection with these hosts. For example, the phage in lambda GEM™-11 may be utilized in making recombinant phage vectors which can be used to transform host cells, such as E. coli LE392.

Promoters commonly used in recombinant DNA construction include the β-lactamase (penicillinase) and lactose promoter systems (Chang et al., 1978: Itakura et al., 1977 Goeddel et al., 1979; Goeddel et al., 1980) and other microbial promoters such as the T7 promoter system (U.S. Pat. No. 4,952,496). Details concerning the nucleotide sequences of promoters are known, enabling a skilled worker to ligate them functionally with genes. The particular promoter used will generally be a matter of choice depending upon the desired results. Hosts that are appropriate for expression of the genes encoding the high molecular weight proteins, fragment analogs or variants thereof, include E. coli, Bacillus species, Haemophilus, fungi, yeast or the baculovirus expression system may be used.

In accordance with this invention, it is preferred to make the high molecular weight proteins by recombinant methods, particularly since the naturally occurring high molecular weight protein as purified from a culture of a species of non-typeable Haemophilus may include trace amounts of toxic materials or other contaminants. This problem can be avoided by using recombinantly produced proteins in heterologous systems which can be isolated from the host in a manner to minimize comtaminants in the purified material. Particularly desirable hosts for expression in this regard include Gram positive bacteria which do not have LPS and are, therefore, endotoxin free. Such hosts include species of Bacillus and may be particularly useful for the production of non-pyrogenic high molecular weight protein, fragments or analogs thereof. Furthermore, recombinant methods of production permit the manufacture of HMW1, HMW2, HMW3 or HMW4, and corresponding HMW proteins from other non-typeable Haemophilus influenzae strains, or fragments thereof, separate from one another and devoid of non-HMW protein of non-typeable Haemophilus influenzae.

Biological Deposits

Certain hybridomas producing monoclonal antibodies specific for high molecular weight protein of Haemophilus influenzae according to aspects of the present invention that are described and referred to herein have been deposited with the American Type Culture Collection (ATCC) located at 12301 Parklawn Drive, Rockville, Md., USA, 20852, pursuant to the Budapest Treaty and prior to the filing of this application. Samples of the deposited hybridomas will become available to the public upon grant of a patent based upon this United States patent application. The invention described and claimed herein is not to be limited in scope by the hybridomas deposited, since the deposited embodiment is intended only as an illustration of the invention. Any equivalent or similar hybridomas that produce similar or equivalent antibodies as described in this application are within the scope of the invention.

Deposit Summary

| Hybridomas | ATCC Designation | Date Deposited |
|---|---|---|
| AD6 | | |
| 10C5 | | |

### EXAMPLES

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations.

Methods of molecular genetics, protein biochemistry, and immunology used but not explicitly described in this disclosure and these Examples are amply reported in the scientific literature and are well within the ability of those skilled in the art.

### Example 1

This Example describes the isolation of DNA encoding HMW1 and HMW2 proteins, cloning and expression of such proteins, and sequencing and sequence analysis of the DNA molecules encoding the HMW1 and HMW2 proteins.

Non-typeable H. influenzae strains 5 and 12 were isolated in pure culture from the middle ear fluid of children with acute otitis media. Chromosomal DNA from strain 12, providing genes encoding proteins HMW1 and HMW2, was prepared by preparing Sau3A partial restriction digests of chromosomal DNA and fractionating on sucrose gradients. Fractions containing DNA fragments in the 9 to 20 kbp range were pooled and a library was prepared by ligation into λEMBL3 arms. Ligation mixtures were packaged in vitro and plate-amplified in a P2 lysogen of E. coli LE392.

For plasmid subcloning studies, DNA from a representative recombinant phage was subcloned into the T7 expression plasmid pT7-7, containing the T7 RNA polymerase promoter Φ10, a ribosome-binding site and the translational start site for the T7 gene 10 protein upstream from a multiple cloning site (see FIG. 5B).

DNA sequence analysis was performed by the dideoxy method and both strands of the HMW1 gene and a single strand of the HMW2 gene were sequenced.

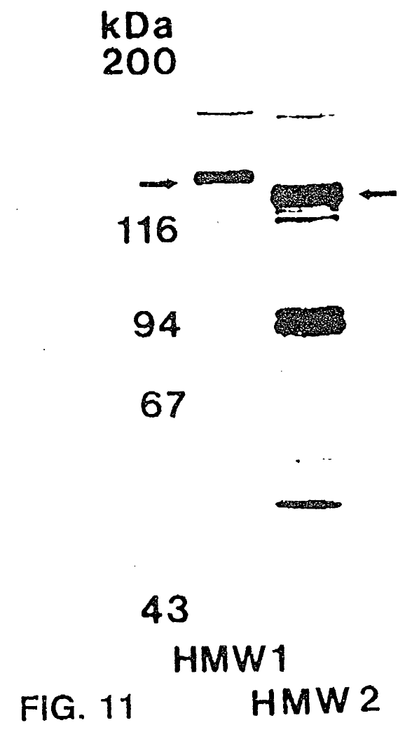

Western immunoblot analysis was performed to identify the recombinant proteins being produced by reactive phage clones (FIG. 11). Phage lysates grown in LE392 cells or plaques picked directly from a lawn of LE392 cells on YT plates were solubilized in gel electrophoresis sample buffer prior to electrophoresis. Sodium dodecyl sulfate polyacrylamide gel electrophoresis (SDS-PAGE) was performed on 7.5% or 11% polyacrylamide modified Laemmli gels. After transfer of the proteins to nitrocellulose sheets, the sheets were probed sequentially with an E. coli-absorbed human serum sample containing high-titer antibody to the high-molecular-weight proteins and then with alkaline phosphatase-conjugated goat anti-human immunoglobulin G (IgG) second antibody. Sera from healthy adults contains high-titer antibody directed against surface-exposed high-molecular-weight proteins of non-typeable H. influenzae. One such serum sample was used as the screening antiserum after having been extensively absorbed with LE392 cells.

To identify recombinant proteins being produced by E. coli transformed with recombinant plasmids, the plasmids of interest were used to transform E. coli BL21 (DE3)/pLysS. The transformed strains were grown to an A₆₀₀ of 0.5 in L broth containing 50 μg of ampicillin per ml. IPTG was then added to 1 mM. One hour later, cells were harvested, and a sonicate of the cells was prepared. The protein concentrations of the samples were determined by the bicinchoninic acid method. Cell sonicates containing 100 μg of total protein were solubilized in electrophoresis sample buffer, subjected to SDS-polyacrylamide gel electrophoresis, and transferred to nitrocellulose. The nitrocellulose was then probed sequentially with the E. coli-absorbed adult serum sample and then with alkaline phosphatase-conjugated goat anti-human IgG second antibody.

Western immunoblot analysis also was performed to determine whether homologous and heterologous non-typeable H. influenzae strains expressed high-molecular-weight proteins antigenically related to the protein encoded by the cloned HMW1 gene (rHMW1). Cell sonicates of bacterial cells were solubilized in electrophoresis sample buffer, subjected to SDS-polyacrylamide gel electrophoresis, and transferred to nitrocellulose. Nitrocellulose was probed sequentially with polyclonal rabbit rHMW1 antiserum and then with alkaline phosphatase-conjugated goat anti-rabbit IgG second antibody.

Finally, Western immunoblot analysis was performed to determine whether non-typeable Haemophilus strains expressed proteins antigenically related to the filamentous hemagglutinin protein of Bordetella pertussis. Monoclonal antibody X3C, a murine immunoglobulin G (IgG) antibody which recognizes filamentous hemagglutinin, was used to probe cell sonicates by Western blot. An alkaline phosphatase-conjugated goat anti-mouse IgG second antibody was used for detection.

To generate recombinant protein antiserum, E. coli BL21(DE3)/pLysS was transformed with pHMW1-4, and expression of recombinant protein was induced with IPTG, as described above. A cell sonicate of the bacterial cells was prepared and separated into a supernatant and pellet fraction by centrifugation at 10,000×g for 30 min. The recombinant protein fractionated with the pellet fraction. A rabbit was subcutaneously immunized on biweekly schedule with 1 mg of protein from the pellet fraction, the first dose given with Freund's complete adjuvant and subsequent doses with Freund's incomplete adjuvant. Following the fourth injection, the rabbit was bled. Prior to use in the Western blot assay, the antiserum was absorbed extensively with sonicates of the host E. coli strain transformed with cloning vector alone.

To assess the sharing of antigenic determinants between HMW1 and filamentous hemagglutinin, enzyme-linked immunosorbent assay (ELISA) plates (Costar, Cambridge, Mass.) were coated with 60 μl of a 4-μg/ml solution of filamentous hemagglutinin in Dulbecco's phosphate-buffered saline per well for 2 h at room temperature. Wells were blocked for 1 h with 1% bovine serum albumin in Dulbecco's phosphate-buffered saline prior to addition of serum dilutions. rHMW1 antiserum was serially diluted in 0.1% Brij (Sigma, St. Louis, Mo.) in Dulbecco's phosphate-buffered saline and incubated for 3 h at room temperature. After being washed, the plates were incubated with peroxidase-conjugated goat anti-rabbit IgG antibody (Bio-Rad) for 2 h at room temperature and subsequently developed with 2,2′-azino-bis(3-ethylbenzthiazoline-6-sulfonic acid) (Sigma) at a concentration of 0.54 in mg/ml in 0.1 M sodium citrate buffer, pH 4.2, containing 0.03% H₂O₂. Absorbances were read on an automated ELISA reader.

Recombinant phage expressing HMW1 or HMW2 were recovered as follows. The non-typeable H. influenzae strain 12 genomic library was screened for clones expressing high-molecular-weight proteins with an E. coli-absorbed human serum sample containing a high titer of antibodies directed against the high-molecular-weight proteins.

Numerous strongly reactive clones were identified along with more weakly reactive ones. Twenty strongly reactive clones were plaque-purified and examined by Western blot for expression of recombinant proteins. Each of the strongly reactive clones expressed one of two types of high-molecular-weight proteins, designated HMW1 and HMW2. The major immunoreactive protein bands in the HMW1 and HMW2 lysates migrated with apparent molecular masses of 125 and 120 kDa, respectively. In addition to the major bands, each lysate contained minor protein bands of higher apparent molecular weight. Protein bands seen in the HMW2 lysates at molecular masses of less than 120 kDa were not regularly observed and presumably represent proteolytic degradation products. Lysates of LE392 infected with the λEMBL3 cloning vector alone were non-reactive when immunologically screened with the same serum sample. Thus, the observed activity was not due to cross-reactive E. coli proteins or XEMBL3-encoded proteins. Furthermore, the recombinant proteins were not simply binding immunoglobulin nonspecifically, since the proteins were not reactive with the goat anti-human IgG conjugate alone, with normal rabbit sera, or with serum from a number of healthy young infants.

Representative clones expressing either the HMW1 or HMW2 recombinant proteins were characterized further. The restriction maps of the two phage types were different from each other, including the regions encoding the HMW1 and HMW2 structural genes. FIG. 5A shows restriction maps of representative recombinant phage which contained the HMW1 or HMW2 structural genes. The locations of the structural genes are indicated by the shaded bars.

HMW1 plasmid subclones were constructed by using the T7 expression plasmid T7-7 (FIGS. 5A and B). HMW2 plasmid subclones also were constructed, and the results with these latter subclones were similar to those observed with the HMW1 constructs.

The approximate location and direction of transcription of the HMW1 structure gene were initially determined by using plasmid pHMW1 (FIG. 5A). This plasmid was constructed by inserting the 8.5-kb BamHI-SalI fragment from λHMW1 into BamHI- and SalI-cut pT7-7. E. coli transformed with pHMW1 expressed an immunoreactive recombinant protein with an apparent molecular mass of 115 kDa, which was strongly inducible with IPTG. This protein was significantly smaller than the 125-kDa major protein expressed by the parent phage, indicating that it either was being expressed as a fusion protein or was truncated at the carboxy terminus.

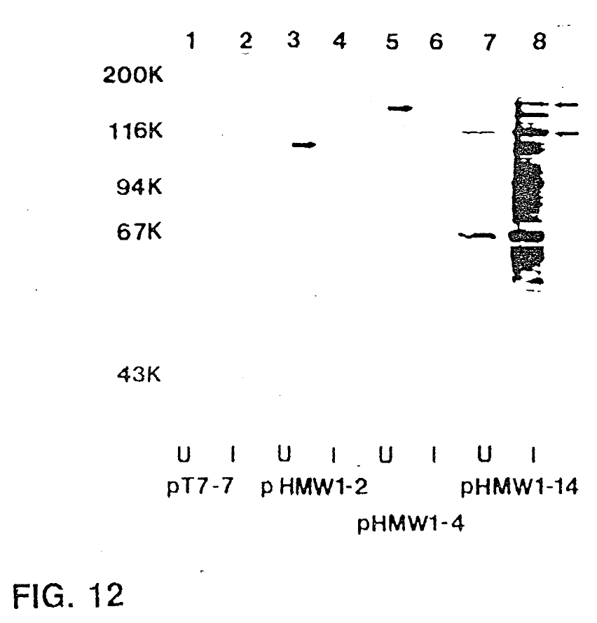

To more precisely localize the 3′ end of the structural gene, additional plasmids were constructed with progressive deletions from the 3′ end of the pHMW1 construct. Plasmid pHMW1-1 was constructed by digestion of pHMW1 with PstI, isolation of the resulting 8.8-kb fragment, and religation. Plasmid pHMW1-2 was constructed by digestion of pHMW1 with HindIII, isolation of the resulting 7.5-kb fragment, and religation. E. coli transformed with either plasmid pHMW1-1 or pHMW1-2 also expressed an immunoreactive recombinant protein with an apparent molecular mass of 115 kDa. These results indicated that the 3′ end of the structural gene was 5′ of the HindIII site. FIG. 12 demonstrates the Western blot results with pHMW1-2 transformed cells before and after IPTG indicates (lanes 3 and 4, respectively). The 115 kDa recombinant protein is indicated by the arrow. Transformants also demonstrated cross-reactive bands of lower apparent molecular weight, and probably represent partial degradation products. Shown for comparison and the results for E. coli transformed with the pT7-7 cloning vector alone (FIG. 12, lanes 1 and 2).

To more precisely localize the 5′ end of the gene, plasmids pHMW1-4 and pHMW1-7 were constructed. Plasmid pHMW1-4 was constructed by cloning the 5.1-kb BamHI-HindIII fragment from λHMW1 into a pT7-7-derived plasmid containing the upstream 3.8-kb EcoRI-BamHi fragment. E. coli transformed with pHMW1-4 expressed an immunoreactive protein with an apparent molecular mass of approximately 160 kDa (FIG. 12, lane 6). Although protein production was inducible with IPTG, the levels of protein production in these transformants were substantially lower than those with the pHMW1-2 transformants described above. Plasmid pHMW1-7 was constructed by digesting pHMW1-4 with NdeI and SpeI. The 9.0-kbp fragment generated by this double digestion was isolated, blunt ended, and religated. E. coli transformed with pHMW1-7 also expressed an immunoreactive protein with an apparent molecular mass of 160 kDa, a protein identical in size to that expressed by the pHMW1-4 transformants. The result indicated that the initiation codon for the HMW1 structural gene was 3′ of the SpeI site. DNA sequence analysis (described below) confirmed this conclusion.

As noted above, the λHMW1 phage clones expressed a major immunoreactive band of 125 kDa, whereas the HMW1 plasmid clones pHMW1-4 and pHMW1-7, which contained what was believed to be the full-length gene, expressed an immunoreactive protein of approximately 160 kDa. This size discrepancy was disconcerting. One possible explanation was that an additional gene or genes necessary for correct processing of the HMW1 gene-product were deleted in the process of subcloning. To address this possibility, plasmid pHMW1-14 was constructed. This construct was generated by digesting pHMW1 with NdeI and MluI and inserting the 7.6-kbp NdeI-MluI fragment isolated from pHMW1-4. Such a construct would contain the full-length HMW1 gene as well as the DNA 3′ of the HMW1 gene which was present in the original HMW1 phage. E. coli transformed with this plasmid expressed major immunoreactive proteins with apparent molecular masses of 125 and 160 kDa as well as additional degradation products (FIG. 12, lanes 7 and 8). The 125- and 160-kDa bands were identical to the major and minor immunoreactive bands detected in the HMW1 phage lysates. Interestingly, the pHMW1-14 construct also expressed significant amounts of protein in the uninduced condition, a situation not observed with the earlier constructs.

The relationship between the 125- and 160-kDa proteins remains somewhat unclear. Sequence analysis, described below, reveals that the HMW1 gene would be predicted to encode a protein of 159 kDa. It is believed that the 160-kDa protein is a precursor form of the mature 125-kDa protein, with the conversion from one protein to the other being dependent on the products of the two downstream genes.

Sequence analysis of the HMW1 gene (FIG. 1) revealed a 4,608-bp open reading frame (ORF), beginning with an ATG codon at nucleotide 351 and ending with a TAG stop codon at nucleotide 4959. A putative ribosome-binding site with the sequence AGGAG begins 10 bp up-stream of the putative initiation codon. Five other in-frame ATG codons are located within 250 bp of the beginning of the ORF, but none of these is preceded by a typical ribosome-binding site. The 5′-flanking region of the ORF contains a series of direct tandem repeats, with the 7-bp sequence ATCTTTC repeated 16 times. These tandem repeats stop 100 bp 5′ of the putative initiation codon. An 8-bp inverted repeat characteristic of a rho-independent transcriptional terminator is present, beginning at nucleotide 4983, 25 bp 3′ of the presumed translational stop. Multiple termination codons are present in all three reading frames both upstream and downstream of the ORF. The derived amino acid sequence of the protein encoded by the HMW1 gene (FIG. 2) has a molecular weight of 159,000, in good agreement with the apparent molecular weights of the proteins expressed by the HMW1-4 and HMW1-7 transformants. The derived amino acid sequence of the amino terminus does not demonstrate the characteristics of a typical signal sequence. The BamHI site used in generation of pHMW1 comprises bp 1743 through 1748 of the nucleotide sequence. The ORF downstream of the BamHI site would be predicted to encode a protein of 111 kDa, in good agreement with the 115 kDa estimated for the apparent molecular mass of the pHMW1-encoded fusion protein.

The sequence of the HMW2 gene (FIG. 3) consists of a 4,431-bp ORF, beginning with an ATG codon at nucleotide 352 and ending with a TAG stop codon at nucleotide 4783. The first 1,259 bp of the ORF of the HMW2 gene are identical to those of the HMW1 gene. Thereafter, the sequences begin to diverge but are 80% identical overall. With the exception of a single base addition at nucleotide 93 of the HMW2 sequence, the 5′-flanking regions of the HMW1 and HMW2 genes are identical for 310 bp upstream from the respective initiation codons. Thus, the HMW2 gene is preceded by the same set of tandem repeats and the same putative ribosome-binding site which lies 5′ of the HMW1 gene. A putative transcriptional terminator identical to that identified 3′ of the HMW1 ORF is noted, beginning at nucleotide 4804. The discrepancy in the lengths of the two genes is principally accounted for by a 186-bp gap in the HMW2 sequence, beginning at nucleotide position 3839. The derived amino acid sequence of the protein encoded by the HMW2 gene (FIG. 4) has a molecular weight of 155,000 and is 71% identical with the derived amino acid sequence of the HMW1 gene.

The derived amino acid sequences of both the HMW1 and HMW2 genes (FIGS. 2 and 4) demonstrated sequence similarity with the derived amino acid sequence of filamentous hemagglutinin of Bordetella pertussis, a surface-associated protein of this organism. The initial and optimized TFASTA scores for the HMW1-filamentous hemagglutinin sequence comparison were 87 and 186, respectively, with a word size of 2. The z score for the comparison was 45.8. The initial and optimized TFASTA scores for the HMW2-filamentous hemagglutinin sequence comparison were 68 and 196, respectively. The z score for the latter comparison was 48.7. The magnitudes of the initial and optimized TFASTA scores and the z scores suggested that a biologically significant relationship existed between the HMW1 and HMW2 gene products and filamentous hemagglutinin. When the derived amino acid sequences of HMW1, HMW2, and filamentous hemagglutinin genes were aligned and compared, the similarities were most notable at the amino-terminal ends of the three sequences. Twelve of the first 22 amino acids in the predicted peptide sequences were identical. In addition, the sequences demonstrated a common five-amino-acid stretch, Asn-Pro-Asn-Gly-Ile, and several shorter stretches of sequence identity within the first 200 amino acids.

### Example 2

This Example describes the relationship of filamentous hemagglutinin and the HMW1 protein.

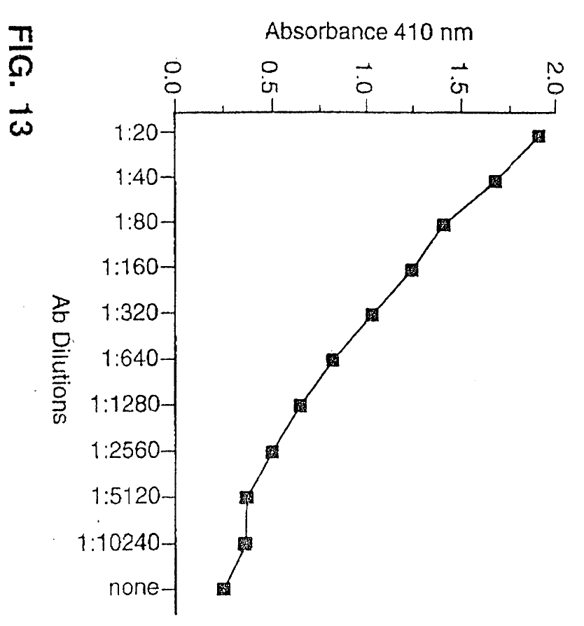

To further explore the HMW1-filamentous hemagglutinin relationship, the ability of antiserum prepared against the HMW1-4 recombinant protein (rHMW1) to recognize purified filamentous hemagglutinin was assessed (FIG. 13). The rHMW1 antiserum demonstrated ELISA reactivity with filamentous hemagglutinin in a dose-dependent manner. Preimmune rabbit serum had minimal reactivity in this assay. The rHMW1 antiserum also was examined in a Western blot assay and demonstrated weak but positive reactivity with purified filamentous hemagglutinin in this system also.

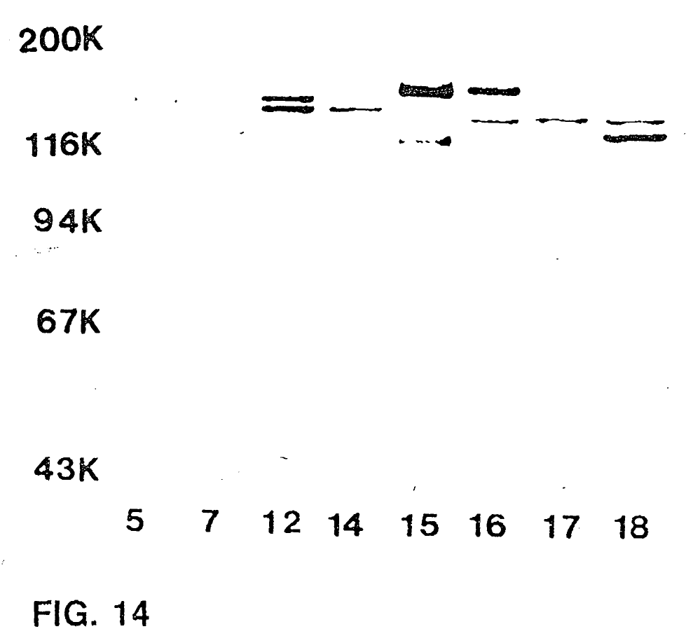

To identify the native Haemophilus protein corresponding to the HMW1 gene product and to determine the extent to which proteins antigenically related to the HMW1 cloned gene product were common among other non-typeable H. influenzae strains, a panel of Haemophilus strains was screened by Western blot with the rHMW1 antiserum. The antiserum recognized both a 125- and a 120-kDa protein band in the homologous strain 12 (FIG. 14), the putative mature protein products of the HMW1 and HMW2 genes, respectively. The 120-kDa protein appears as a single band in FIG. 14, wherein it appeared as a doublet in the HMW2 phage lysates (FIG. 11).

When used to screen heterologous non-typeable H. influenzae strains, rHMW1 antiserum recognized high-molecular-weight proteins in 75% of 125 epidemiologically unrelated strains. In general, the antiserum reacted with one or two protein bands in the 100- to 150-kDa range in each of the heterologous strains in a pattern similar but not identical to that seen in the homologous strain (FIG. 14).

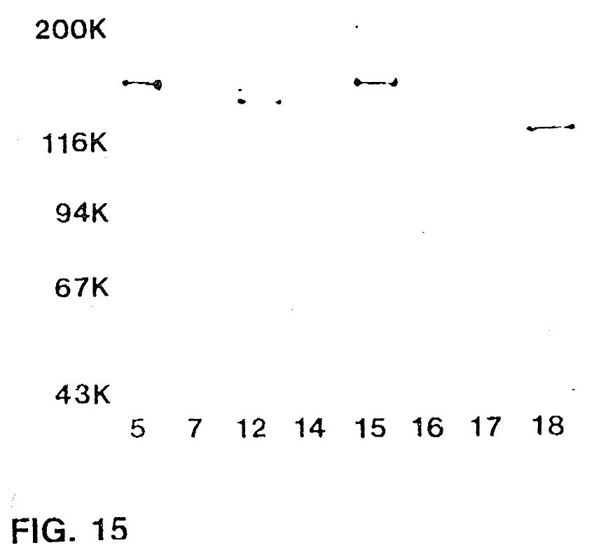

Monoclonal antibody X3C is a murine IgG antibody directed against the filamentous hemagglutinin protein of B. pertussis. This antibody can inhibit the binding of B. pertussis cells to Chinese hamster ovary cells and HeLa cells in culture and will inhibit hemagglutination of erythrocytes by purified filamentous hemagglutinin. A Western blot assay was performed in which this monoclonal antibody was screened against the same panel of non-typeable H. influenzae strains discussed above (FIG. 14). Monoclonal antibody X3C recognized both the high-molecular-weight proteins in non-typeable H. influenzae strain 12 which were recognized by the recombinant-protein antiserum (FIG. 15). In addition, the monoclonal antibody recognized protein bands in a subset of heterologous non-typeable H. influenzae strains which were identical to those recognized by the recombinant-protein antiserum, as may be seen by comparison of FIGS. 14 and 15. On occasion, the filamentous hemagglutinin monoclonal antibody appeared to recognize only one of the two bands which had been recognized by the recombinant-protein antiserum (compare strain lane 18 in FIGS. 14 and 15, for example). Overall, monoclonal antibody X3C recognized high-molecular-weight protein bands identical to those recognized by the rHMW1 antiserum in approximately 35% of our collection of non-typeable H. influenzae strains.

### Example 3

This Example describes the adhesin properties of the HMW1 and HMW2 proteins.

Mutants deficient in expression of HMW1, HMW2 or both proteins were constructed to examine the role of these proteins in bacterial adherence. The following strategy was employed. pHMW1-14 (see Example 1, FIG. 5A) was digested with BamHI and then ligated to a kanamycin cassette isolated on a 1.3-kb BamHl fragment from pUC4K. The resultant plasmid (pHMW1-17) was linearized by digestion with XbaI and transformed into non-typeable H. influenzae strain 12, followed by selection for kanamycin resistant colonies. Southern analysis of a series of these colonies demonstrated two populations of transformants, one with an insertion in the HMW1 structural gene and the other with an insertion in the HMW2 structural gene. One mutant from each of these classes was selected for further studies.

Mutants deficient in expression of both proteins-were recovered using the following protocol. After deletion of the 2.1-kb fragment of DNA between two EcoRI sites spanning the 3′-portion of the HMW1 structural gene and the 5′-portion of a downstream gene encoding an accessory processing protein in. pHMW-15, the kanamycin cassette from pUC4K was inserted as a 1.3-kb EcoRI fragment. The resulting plasmid (pHMW1-16) was linearized by digestion with XbaI and transformed into strain 12, followed again by selection for kanamycin resistant colonies. Southern analysis of a representative sampling of these colonies demonstrated that in seven of eight cases, insertion into both the HMW1 and HMW2 loci had occurred. One such mutant was selected for further studies.

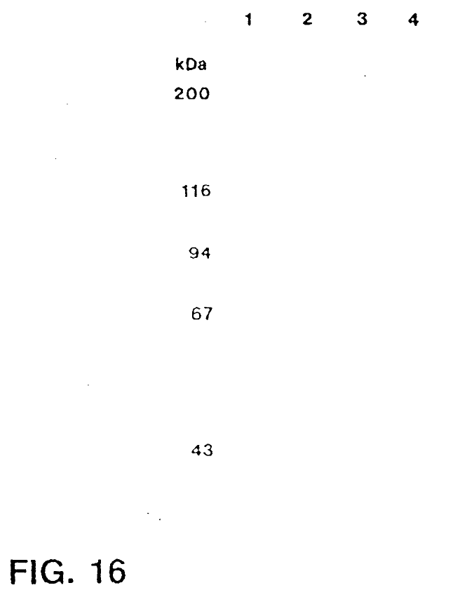

To confirm the intended phenotypes, the mutant strains were examined by Western blot analysis with a polyclonal antiserum against recombinant HMW1 protein. The parental strain expressed both the 125-kD HMW1 and the 120-kD HMW2 protein (FIG. 16). In contrast, the HMW2⁻ mutant failed to express the 120-kD protein, and the HMW1 mutant failed to express the 125-kD protein. The double mutant lacked expression of either protein. On the basis of whole cell lysates, outer membrane profiles, and colony morphology, the wild type strain and the mutants were otherwise identical with one another. Transmission electron microscopy demonstrated that none of the four strains expressed pili.

The capacity of wild type strain 12 to adhere to Chang epithelial cells was examined. In such assays, bacteria were inoculated into broth and allowed to grow to a density of ˜2×10⁹ cfu/ml. Approximately 2×10⁷ cfu were inoculated onto epithelial cell monolayers, and plates were gently centrifuged at 165×g for 5 minutes to facilitate contact between bacteria and the epithelial surface. After incubation for 30 minutes at 37° C. in 5% CO₂, monolayers were rinsed 5 times with PBS to remove nonadherent organisms and were treated with trypsin-EDTA (0.05% trypsin, 0.5% EDTA) in PBS to release them from the plastic support. Well contents were agitated, and dilutions were plated on solid medium to yield the number of adherent bacteria per monolayer. Percent adherence was calculated by dividing the number of adherent cfu per monolayer by the number of inoculated cfu.

As depicted in Table 1 below (the Tables appear at the end of the descriptive text), this strain adhered quite efficiently, with nearly 90% of the inoculum binding to the monolayer. Adherence by the mutant expressing HMW1 but not HMW2 (HMW2⁻) was also quite efficient and comparable to that by the wild type strain. In contrast, attachment by the strain expressing HMW2 but deficient in expression of HMW1 (HMW1⁻) was decreased about 15-fold relative to the wild type. Adherence by the double mutant (HMW1⁻/HMW2⁻) was decreased even further, approximately 50-fold compared with the wild type and approximately 3-fold compared with the HMW1 mutant. Considered together, these results suggest that both the HMW1 protein and the, HMW2 protein influence attachment to Chang epithelial cells. Interestingly, optimal adherence to this cell line appears to require HMW1 but not HMW2.

### Example 4

This Example illustrates the preparation and expression of HMW3 and HMW4 proteins and their function as adhesins.

Using the plasmids pHMW1-16 and pHMW1-17 (see Example 3) and following a scheme similar to that employed with strain 12 as described in Example 3, three non-typeable Haemophilus strain 5 mutants were isolated, including one with the kanamycin gene inserted into the hmw1-like (designated hmw3) locus, a second with an insertion in the hmw2-like (designated hmw4) locus, and a third with insertions in both loci. As predicted, Western immunoblot analysis demonstrated that the mutant with insertion of the kanamycin cassette into the hmw1-like locus had lost expression of the HMW3 125-kD protein, while the mutant with insertion into the hmw2-like locus failed to express the HMW4 123-kD protein. The mutant with a double insertion was unable to express either of the high molecular weight proteins.

As shown in Table 1 below, wild type strain 5 demonstrated high level adherence, with almost 80% of the inoculum adhering per monolayer. Adherence by the mutant deficient in expression of the HMW2-like protein (i.e. HMW4 protein) was also quite high. In contrast, adherence by the mutant unable to express the HMW1-like protein (i.e. HMW3 protein) was reduced about 5-fold relative to the wild type, and attachment by the double mutant was diminished even further (approximately 25-fold). Examination of Giemsa-stained samples confirmed these observations (not shown). Thus, the results with strain 5 for proteins HMW3 and HMW4 corroborate the findings with strain 12 and the HMW1 and HMW2 proteins.

### Example 5

This Example contains additional data concerning the adhesin properties of the HMW1 and HMW2 proteins.

To confirm an adherence function for the HMW1 and HMW2 proteins and to examine the effect of HMW1 and HMW2 independently of other H. influenzae surface structures, the hmw1 and the hmw2 gene clusters were introduced into E. coli DH5α, using plasmids pHMW1-14 and pHMW2-21, respectively. As a control, the cloning vector, pT7-7, was also transformed into E. coli DH5α. Western blot analysis demonstrated that E. coli DH5α containing the hmw1 genes expressed a 125 kDa protein, while the same strain harboring the hmw2 genes expressed a 120-kDa protein. E. coli DH5α containing pT7-7 failed to react with antiserum against recombinant HMW1. Transmission electron microscopy revealed no pili or other surface appendages on any of the E. coli strains.

Adherence by the E. coli strains was quantitated and compared with adherence by wild type non-typeable H. influenzae strain 12. As shown in Table 2 below, adherence by E. coli DH5α containing vector alone was less than 1% of that for strain 12. In contrast, E. coli DH5α harboring the hmw1 gene cluster demonstrated adherence levels comparable to those for strain 12. Adherence by E. coli DH5α containing the hmw2 genes was approximately 6-fold lower than attachment by strain 12 but was increased 20-fold over adherence by E. coli DH5α with pT7-7 alone. These results indicate that the HMW1 and HMW2 proteins are capable of independently mediating attachment to Chang conjunctival cells. These results are consistent with the results with the H. influenzae mutants reported in Examples 3 and 4, providing further evidence that, with Chang epithelial cells, HMW1 is a more efficient adhesin than is HMW2.

Experiments with E. coli HB101 harboring pT7-7, pHMW1-14, or pHMW2-21 confirmed the results obtained with the DH5α derivatives (see Table 2).

### Example 6

This Example illustrates the copurification of HMW1 and HMW2 proteins from wild-type non-typeable H. influenzae strain.

HMW1 and HMW2 were isolated and purified from non-typeable H. influenzae (NTHI) strain 12 in the following manner. Non-typeable Haemophilus bacteria from frozen stock culture were streaked onto a chocolate plate and grown overnight at 37° C. in an incubator with 5% CO₂. 50 ml starter culture of brain heart infusion (BHI) broth, supplemented with 10 μg/ml each of hemin and NAD was inoculated with growth on chocolate plate. The starter culture was grown until the optical density (O.D.-600 nm) reached 0.6 to 0.8 and then the bacteria in the starter culture was used to inoculate six 500 ml flasks of supplemented BHI using 8 to 10 ml per flask. The bacteria were grown in 500 ml flasks for an additional 5 to 6 hours at which time the O.D. was 1.5 or greater. Cultures were centrifuged at 10,000 rpm for 10 minutes.

Bacterial pellets were resuspended in a total volume of 250 ml of an extraction solution comprising 0.5 M NaCl, 0.01 M Na₂EDTA, 0.01 M Tris 50 μM 1,10-phenanthroline, pH 7.5. The cells were not sonicated or otherwise disrupted. The resuspended cells were allowed to sit on ice at 0° C. for 60 minutes. The resuspended cells were centrifuged at 10,000 rpm for 10 minutes at 4° C. to remove the majority of intact cells and cellular debris. The supernatant was collected and centrifuged at 100,000×g for 60 minutes at 4° C. The supernatant again was collected and dialyzed overnight at 4° C. against 0.01 M sodium phosphate, pH 6.0.

The sample was centrifuged at 10,000 rpm for 10 minutes at 4° C. to remove insoluble debris precipitated from solution during dialysis. The supernatant was applied to a 10 ml CM Sepharose column which has been pre-equilibrated with 0.01 M sodium phosphate, pH 6. Following application to this column, the column was washed with 0.01 M sodium phosphate. Proteins were elevated from the column with a 0-0.5M KCl gradient in 0.01 M Na phosphate, pH 6 and fractions were collected for gel examination. Coomassie gels of column fractions were carried out to identify those fractions containing high molecular weight proteins. The fractions containing high molecular weight proteins were pooled and concentrated to a 1 to 3 ml volume in preparation for application of sample to gel filtration column.

A Sepharose CL-4B gel filtration column was equilibrated with phosphate-buffered saline, pH 7.5. The concentrated high molecular weight protein sample was applied to the gel filtration column and column fractions were collected. Coomassie gels were performed on the column fractions to identify those containing high molecular weight proteins. The column fractions containing high molecular weight proteins were pooled.

### Example 7

This Example illustrates the use of specified HMW1 and HMW2 proteins in immunization studies.

The copurified HMW1 and HMW2 proteins prepared as described in Example 6 were tested to determine whether they would protect against experimental otitis media caused by the homologous strain.

Healthy adult chinchillas, 1 to 2 years of age with weights of 350 to 500 g, received three monthly subcutaneous injections with 40 μg of an HMW1-HMW2 protein mixture in Freund's adjuvant. Control animals received phosphate-buffered saline in Freunds' adjuvant. One month after the last injection, the animals were challenged by intrabullar inoculation with 300 cfu of NTHI strain 12.

Middle ear infection developed in 5 of 5 control animals versus 5 of 10 immunized animals. Although only 5 of 10 chinchillas were protected in this test, the test conditions are very stringent, requiring bacteria to be injected directly into the middle ear space and to proliferate in what is in essence a small abscess cavity. As seen from the additional data below, complete protection of chinchillas can be achieved.

The five HMW1/HMW2-immunized animals that did not develop otitis media demonstrated no signs of middle ear inflammation when examined by otoscopy nor were middle ear effusions detectable.

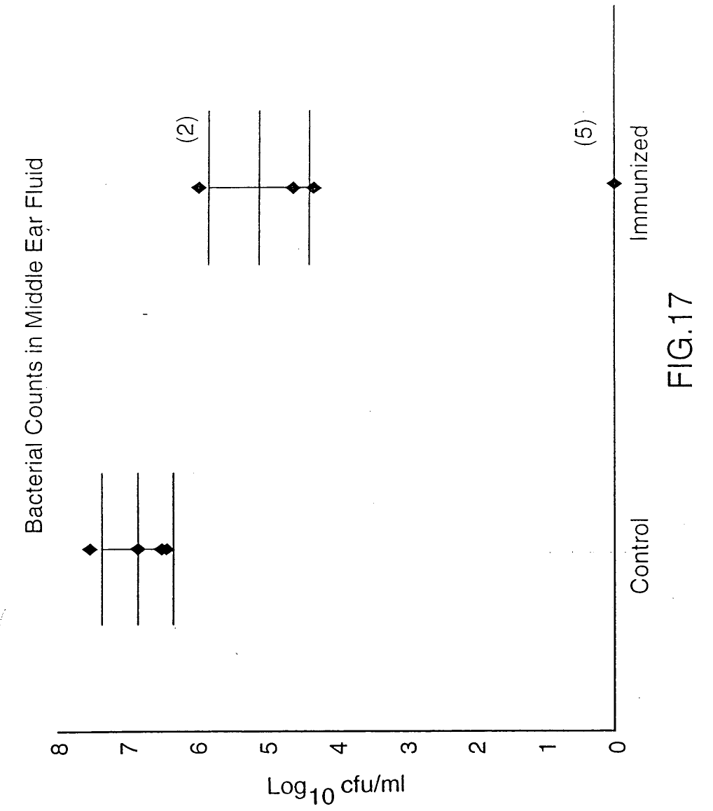

Among the five HMW1/HMW2-immunized animals that became infected, the total duration of middle ear infection as assessed by the persistence of culture-positive middle ear fluid was not different from controls. However, the degree of inflammation of the tympanic membranes was subjectively less than in the HMW1/HMW2-immunized animals. When quantitative bacterial counts were performed on the middle ear fluid specimens recovered from infected animals, notable differences were apparent between the HMW1/HMW2-immunized and PBS-immunized animals (FIG. 17). Shown in FIG. 17 are quantitative middle ear fluid bacterial counts from animals on day 7 post-challenge, a time point associated with the maximum colony counts in middle ear fluid. The data were log-transformed for purpose of statistical comparison. The data from the control animals are shown on the left and data from the high molecular weight protein immunized animals on the right. The two horizontal lines indicate the respective means and standard derivations of middle ear fluid colony counts for only the infected animals in each group. As can be seen from this Figure, the HMW1/HMW2-immunized animals had significantly lower middle ear fluid bacterial counts than the PBS-immunized controls, geometric means of 7.4×10⁶ and 1.3×10⁵, respectively (p=0.02, Students' t-test)

Serum antibody titres following immunization were comparable in uninfected and infected animals. However, infection in immunized animals was uniformly associated with the appearance of bacteria down-regulated in expression of the HMW proteins, suggesting bacterial selection in response to immunologic pressure.

Although this data shows that protection following immunization was not complete, this data suggests the HMW adhesin proteins are potentially important protective antigens which may comprise one component of a multi-component NTHI vaccine.

In addition, complete protection has been achieved in the chinchilla model at lower dosage challenge, as set forth in Table 3 below.

Groups of five animals were immunized with 20 μg of the HMW1-HMW2 mixture prepared as described in Example 6 on days 1, 28 and 42 in the presence of alum. Blood samples were collected on day 53 to monitor the antibody response. On day 56, the left ear of animals was challenged with about 10 cfu of H. influenzae strain 12. Ear infection was monitored on day 4. Four animals in Group 3 were infected previously by H. influenzae strain 12 and were recovered completely for at least one month before the second challenge.

### Example 8

This Example illustrates the provision of synthetic peptides corresponding to a portion only of the HMW1 protein.

A number of synthetic peptides were derived from HMW1 Antisera then were raised to these peptides. The anti-peptide antisera to peptide HMW1-P5 was shown to recognize HMW1. Peptide HMW1-P5 covers amino acids 1453 to 1481 of HMW1, has the sequence VDEVIEAKRILEKVKDLSDEEREALAKLG (SEQ ID No: 11), and represents bases 1498 to 1576 in FIG. 10.

This finding demonstrates that the DNA sequence and the derived protein is being interpreted in the correct reading frame and that peptides derived from the sequence can be produced which will be immunogenic.

### Example 9

This Example describes the generation of monoclonal antibodies to the high molecular weight proteins of non-typeable H. influenzae.

Monoclonal antibodies were generated using standard techniques. In brief, female BALB/c mice (4 to 6 weeks old) were immunized by intraperitoneal injection with high molecular weight proteins purified from nontypable Haemophilus strain 5 or strain 12, as described in Example 6. The first injection of 40 to 50 μg of protein was administered with Freund's complete adjuvant and the second dose, received four to five weeks after the first, was administered with phosphate-buffered saline. Three days following the second injection, the mice were sacrificed and splenic lymphocytes were fused with SP2/0-Agl4 plasmacytoma cells.

Two weeks following fusion, hybridoma supernatants were screened for the presence of high molecular weight protein specific antibodies by a dot-blot assay. Purified high molecular weight proteins at a concentration of 10 μg per ml in TRIS-buffered saline (TBS), were used to sensitize nitrocellulose sheets (Bio-Rad Laboratories, Richmond, Calif.) by soaking for 20 minutes. Following a blocking step with TBS-3% gelatin, the nitrocellulose was incubated for 60 minutes at room temperature with individual hybridoma supernatants, at a 1:5 dilution in TBS-0.1% Tween, using a 96-well Bio-Dot micro-filtration apparatus (Bio-Rad). After washing, the sheets were incubated for one hour with alkaline-phosphatase-conjugated affinity isolated goat-anti (mouse IgG+IgM) antibodies (Tago, Inc., Burlingame, Calif.). Following additional washes, positive supernatants were identified by incubation of the nitrocellulose sheet in alkaline phosphatase buffer (0.10 M TRIS, 0.10 M NaCl, 0.005 M MgCl₂,) containing nitroblue tetrazolium (0.1 mg/ml) and 5-bromo-4-chloro-3-indoyl phosphate (BCIP) (0.05 mg/ml).

For the antibody isotyping and immunoelectron microscopy studies to be described below, the monoclonal antibodies were purified from hybridoma supernatants. The antibodies recovered in this work were all of the IgG class. To purify the monoclonal antibodies, the hybridoma supernatants were first subjected to ammonium sulfate precipitation (50% final concentration at 0° C.). Following overnight incubation, the precipitate was recovered by centrifugation and resolubilized in phosphate buffered saline. The solution was then dialyzed overnight against 0.01 M sodium phosphate buffer, pH 6.0. The following day the sample was applied to a DEAE-Sephacel column preequilibrated with the same phosphate buffer and the proteins were subsequently eluted with a KCl gradient. Column fractions containing the monoclonal antibodies were identified by examination of samples on Coomassie gels for protein bands typical of light and heavy chains.

The isotype of each monoclonal antibody was determined by immunodiffusion using the Ouchterlony method. Immunodiffusion plates were prepared on glass slides with 10 ml of 1% DNA-grade agarose (FMC Bioproducts, Rockland, Me.) in phospate-buffered saline. After the agarose solidified, 5-mm wells were punched into the agarose in a circular pattern. The center well contained a concentrated preparation of the monoclonal antibody being evaluated and the surrounding wells contained goat anti-mouse subclass-specific antibodies (Tago). The plates were incubated for 48 hours in a humid chamber at 4° C. and then examined for white lines of immunoprecipitation.

Hybridoma supernatants which were reactive in the dot-blot assay described above were examined by Western blot analysis, both to confirm the reactivity with the high molecular weight proteins of the homologous nontypable Haemophilus strain and to examine the cross-reactivity with similar proteins in heterologous strains. Nontypable Haemophilus influenzae cell sonicates containing 100 μg of total protein were solubilized in electrophoresis sample buffer, subjected to SDS-polyacrylamide gel electrophoresis on 7.5% acrylamide gels, and transferred to nitrocellulose using a Genie electrophoretic blotter (Idea Scientific Company, Corvallis, Oreg.) for 45 min at 24 V. After transfer, the nitrocellulose sheet was blocked and then probed sequentially with the hybridoma supernatant, with alkaline phosphatase-conjugated goat-anti (mouse IgG+IgM) second antibody, and finally bound antibodies were detected by incubation with nitroblue tetrazolium/BCIP solution. This same assay was employed to examine the reactivity of the monoclonals with recombinant fusion proteins expressed in E. coli (see below).

In preparation for immunoelectronmicroscopy, bacteria were grown overnight on supplemented chocolate agar and several colonies were suspended in phosphate-buffered-saline containing 1% albumin. A 20-μl drop of this bacterial suspension was then applied to a carbon-coated grid and incubated for 2 min. Excess fluid was removed and the specimen was then incubated for 5 min with the purified high molecular weight protein-specific monoclonal antibody being analyzed. Following removal of excess liquid and a wash with phosphatebuffered saline, the specimen was incubated with anti-mouse IgG conjugated to 10-nm colloidal gold particles. Following final washes with phosphate-buffered saline, the sample was rinsed with distilled water. Staining of the bacterial cells was performed with 0.5% uranyl acetate for 1 min. Samples were then examined in a Phillips 201c electron microscope.

Fourteen different hybridomas were recovered which produced monoclonal antibodies reactive with the purified HMW1 and HMW2 proteins of nontypable Haemophilus strain 12 in the immunoblot screening assay. Of the monoclonals screened by immunoelectron microscopy to date, as described below, two were demonstrated to bind surface epitopes on prototype strain 12. These two monoclonal antibodies, designated AD6 (ATCC ______) and 10C5 (ATCC ______), were both of the IgG1 subclass.

### Example 10

This Example describes the identification of surface-exposed B-cell epitopes of high molecular weight proteins of non-typeable H. influenzae.

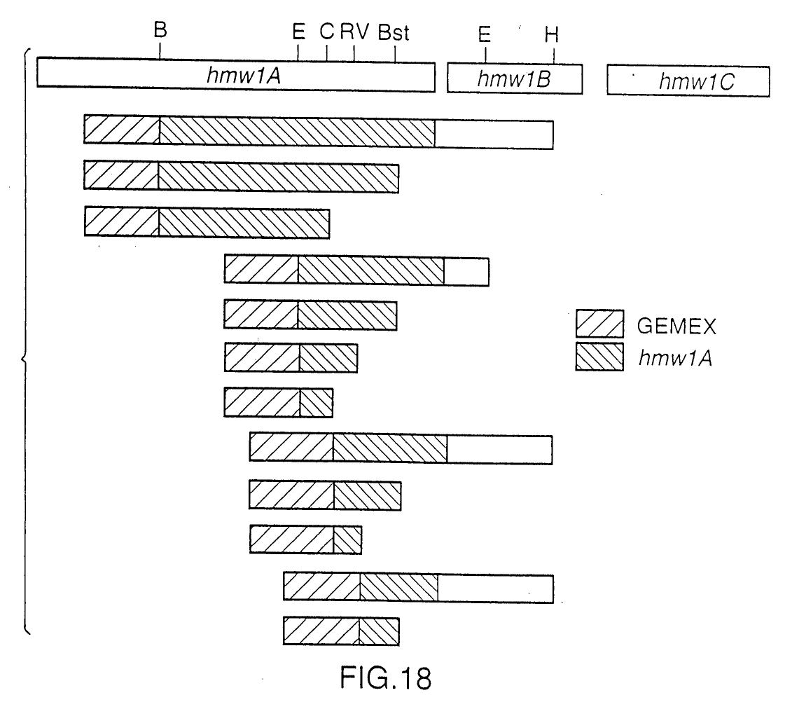

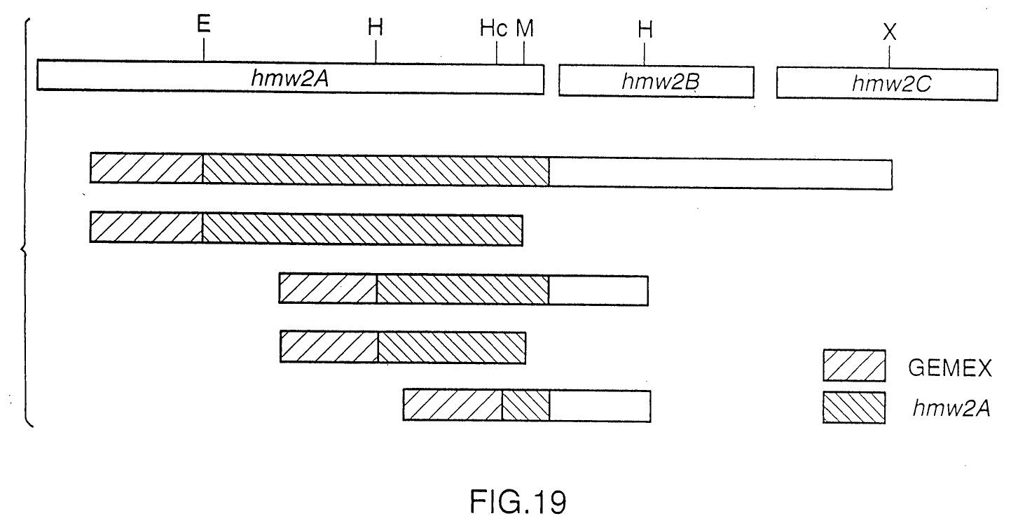

To map epitopes recognized by the monoclonal antibodies, their reactivity with a panel of recombinant fusion proteins expressed by pGEMEX® recombinant plasmids was examined. These plasmids were constructed by cloning various segments of the hmw1a or hmw2A structural genes into T7 expression vectors pGEMEX®-1 and GEMEX®-2 (Promega Corporation, Madison, Wis.). Shown in FIGS. 18 and 19 are the schematic diagrams depicting the segments derived from the hmw1 and hmw2 gene clusters cloned into the pGEMEX® expression plasmids. These segments were inserted such that in-frame fusions were created at each junction site. Thus, these plasmids encode recombinant fusion proteins containing pGEMEX®-encoded T7 gene 1.0 amino acids in the regions indicated by the hatched bars and hmw1a or hmw2A encoded amino acids in the regions indicated by the black bars in these Figures. A stop codon is present at the junction of the black and white segments of each bar.

Four discrete sites within the hmw1A structural gene were selected as the 5′ ends of the hmw1 inserts. For each 5′ end, a series of progressively smaller inserts was created by taking advantage of convenient downstream restriction sites. The first recombinant plasmid depicted in FIG. 18 was constructed by isolating a 4.9 kbp BamHI-HindIII fragment from pHMW1-14 (Example 1, FIG. 5A), which contains the entire hmw1 gene cluster and inserting it into BamHI-HindIII digested pGEMEX®-1. The second recombinant plasmid in this set was constructed by digesting the “parent” plasmid with BstEII-HindIII, recovering the 6.8 kbp larger fragment, blunt-ending with Klenow DNA polymerase, and religating. The third recombinant plasmid in this set was constructed by digesting the “parent” plasmid with ClaI-HindIII, recovering the 6.0 kbp larger fragment, blunt-ending, and religating. The next set of four hmw1 recombinant plasmids was derived from a “parent” plasmid constructed by ligating a 2.2 kbp EcoRI fragment from the hmw1 gene cluster into EcoRI-digested pGEMEX®-2. The other three recombinant plasmids in this second set were constructed by digesting at downstream BstEII, EcoRV, and ClaI sites, respectively, using techniques similar to those just described. The third set of three recombinant plasmids depicted was derived from a “parent” plasmid constructed by double-digesting the first recombinant plasmid described above (i.e. the one containing the 4.9 kbp BamHI-HindIII fragment) with BamHI and ClaI, blunt-ending, and religating. This resulted in a construct encoding a recombinant protein with an in-frame fusion at the ClaI site of the hmw1A gene. The remaining two plasmids in this third set were constructed by digesting at downstream BstEII and EcoRV sites, respectively. Finally, the fourth set of two recombinant plasmids was derived from a “parent” plasmid constructed by double-digesting the original BamHI-HindIII construct with HincII and EcoRV, then religating. This resulted in a construct encoding a recombinant protein with an in-frame fusion at the EcoRV site of the hmw1A gene. The remaining plasmid in this fourth set was constructed by digesting at the downstream BstEII site.

Three discrete sites with the hmw2A structural gene were selected as the 5′ ends of the hmw2 inserts. The first recombinant plasmid depicted in FIG. 19 was constructed by isolating a 6.0 kbp EcoRI-XhoI fragment from pHMW2-21, which contains the entire hmw2 gene cluster, and inserting it into EcoRI-SalI digested pGEMEX®-1. The second recombinant plasmid in this set was constructed by digesting at an MluI site near the 3′ end of the hmw2A gene. The second set of two hmw2 recombinant plasmids was derived from a “parent” plasmid constructed by isolating a 2.3 kbp HindIII fragment from pHMW2-21 and inserting it into HindIII-digested pGEMEX®-2. The remaining plasmid in this second set was constructed by digesting at the downstream MluI site. Finally, the last plasmid depicted was constructed by isolating a 1.2 kbp HincII-HindIII fragment from the indicated location in the hmw2 gene cluster and inserting it into HincII-HindIII digested pGEMEX®-1.

Each of the recombinant plasmids was used to transform E. coli strain JM101. The resulting transformants were used to generate the recombinant fusion proteins employed in the mapping studies. To prepare recombinant proteins, the transformed E. coli strains were grown to an A₆₀₀ of 0.5 in L broth containing 50 μg of ampicillin per ml. IPTG was then added to 1 mM and mGPl-2, the M13′ phage containing the T7 RNA polymerase gene, was added at multiplicity of infection of 10. One hour later, cells were harvested, and a sonicate of the cells was prepared. The protein concentrations of the samples were determined and cell sonicates containing 100 μg of total protein were solubilized in electrophoresis sample buffer, subjected to SDS-polyacrylamide gel electrophoresis, and examined on Coomassie gels to assess the expression level of recombinant fusion proteins. Once high levels of expression of the recombinant fusion proteins were confirmed, the cell sonicates were used in the Western blot analyses described above.

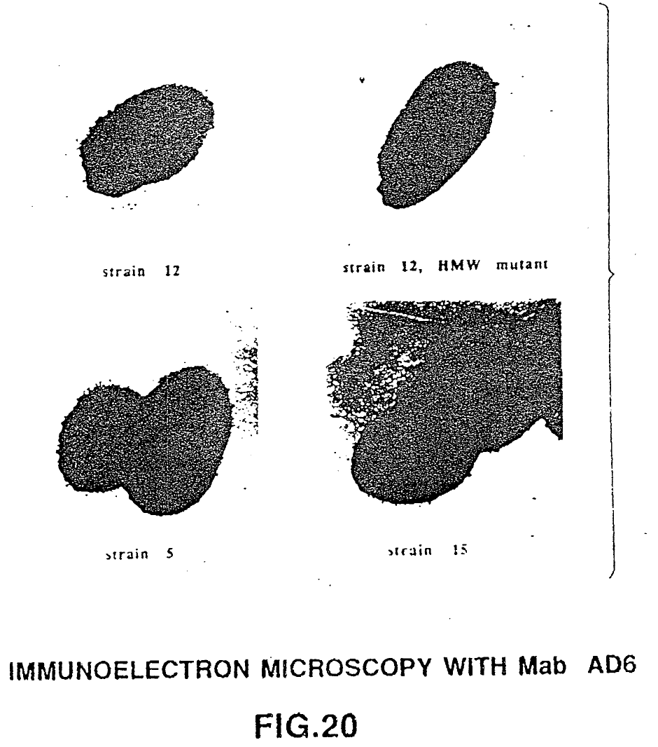

Shown in FIG. 20 is an electron micrograph demonstrating surface binding of Mab AD6 to representative nontypable Haemophilus influenzae strains. In the upper left panel of the Figure is nontypable Haemophilus strain 12 and in the upper right panel is a strain 12 derivative which no longer expressed the high molecular weight proteins. As can be seen, colloidal gold particles decorate the surface of strain 12, indicating bound AD6 antibody on the surface. In contrast, no gold particles are evident on the surface of the strain 12 mutant which no longer expresses the high molecular weight proteins. These results indicate that monoclonal antibody AD6 is recognizing a surface-exposed epitope on the high molecular weight proteins of strain 12. Analogous studies were performed with monoclonal antibody 10C5 demonstrating it too bound to surface-accessible epitopes on the high molecular weight HMW1 and HMW2 proteins of strain 12.

Having identified two surface-binding monoclonals, the epitope which each monoclonal recognized was mapped. To accomplish this task, the two sets of recombinant plasmids containing various portions of either the hmw1a or hmw2A structural genes (FIGS. 18 and 19) were employed. With these complementary sets of recombinant plasmids, the epitopes recognized by the monoclonal antibodies were mapped to relatively small regions of the very large HMW1 and HMW2 proteins.

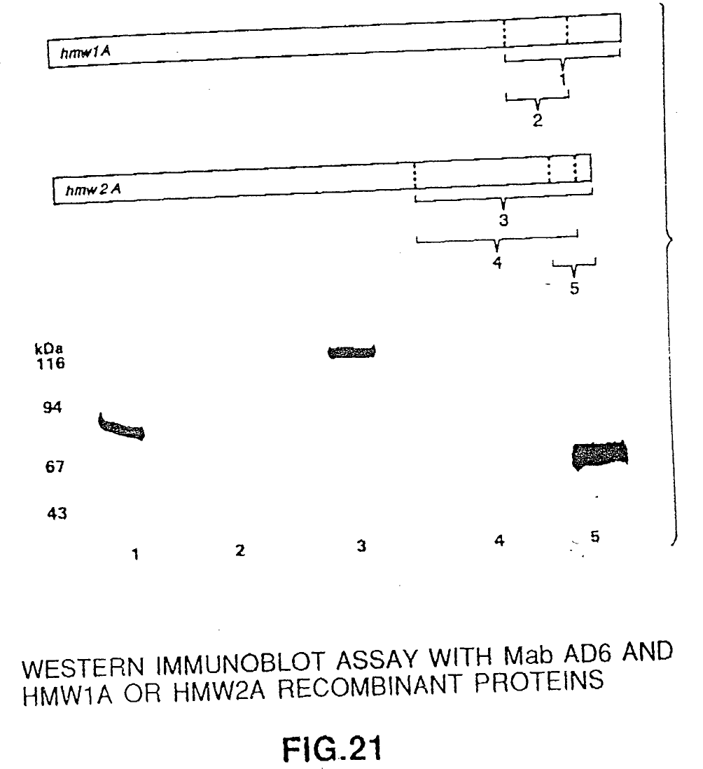

To localize epitopes recognized by Mab AD6, the pattern of reactivity of this monoclonal antibody with a large set of recombinant fusion protein was examined. FIG. 21 is a Western blot which demonstrates the pattern of reactivity of Mab AD6 with five recombinant fusion proteins, a relevant subset of the larger number originally examined. From analysis of the pattern of reactivity of Mab AD6 with this set of proteins, one is able to map the epitope it recognizes to a very short segment of the HMW1 and HMW2 proteins. A brief summary of this analysis follows. For reference, the relevant portions of the hmw1A or hmw2A structural genes which were expressed in the recombinant proteins being examined are indicated in the diagram at the top of the figure. As shown in lane 1, Mab AD6 recognizes an epitope encoded by fragment 1, a fragment which encompasses the distal one-fourth of the hmw1A gene. Reactivity is lost when only the portion of the gene comprising fragment 2 is expressed. This observation localizes the AD6 epitope somewhere within the last 180 amino acids at the carboxy-terminal end of the HMW1 protein. Mab AD6 also recognizes an epitope encoded by fragment 3, derived from the hmw2A structural gene. This is a rather large fragment which encompasses nearly one-third of the gene. Reactivity is lost when fragment 4 is expressed. The only difference between fragments 3 and 4 is that the last 225 base pairs at the 3′ end of the hmw2A structural gene were deleted in the latter construct. This observation indicates that the AD6 epitope is encoded by this short terminal segment of the hmw2A gene. Strong support for this idea is provided by the demonstrated binding of Mab AD6 to the recombinant protein encoded by fragment 5, a fragment encompassing the distal one-tenth of the hmw2A structural gene. Taken together, these data identify the AD6 epitope as common to both the HMW1 and HMW2 proteins and place its location with 75 amino acids of the carboxy termini of the two proteins.

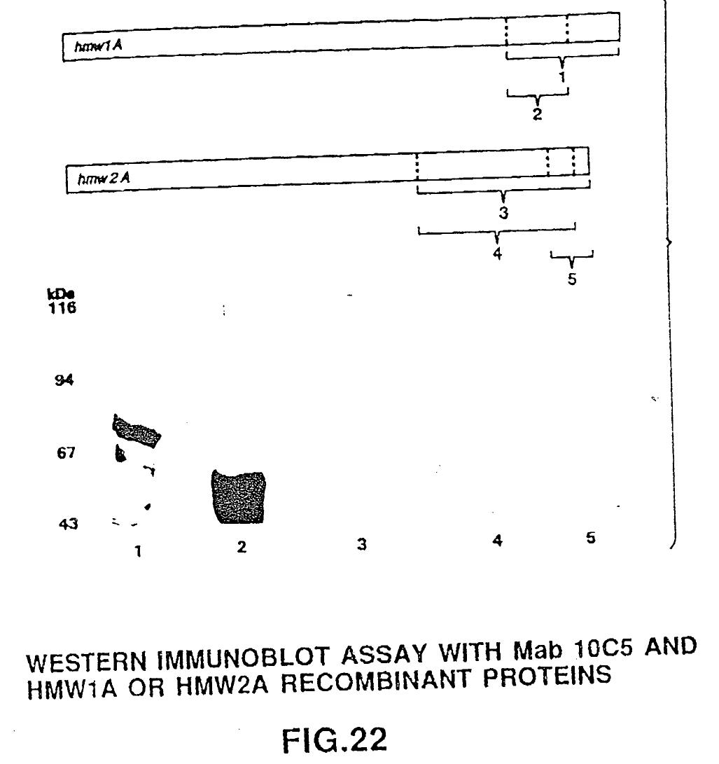

FIG. 22 is a Western blot demonstrating the pattern of reactivity of Mab 10C5 with the same five recombinant fusion proteins examined in FIG. 21. As shown in lane 1, Mab 10C5 recognizes an epitope encoded by fragment 1. In contrast to Mab AD6, Mab 10C5 also recognizes an epitope encoded by fragment 2. Also in contrast to Mab AD6, Mab 10C5 does not recognize any of the hmw2A-derived recombinant fusion proteins. Thus, these data identify the 10C5 epitope as being unique to the HMW1 protein and as being encoded by the fragment designated as fragment 2 in this figure. This fragment corresponds to a 155-amino acid segment encoded by the EcoRV-BstEII segment of the hmw1A structural gene.

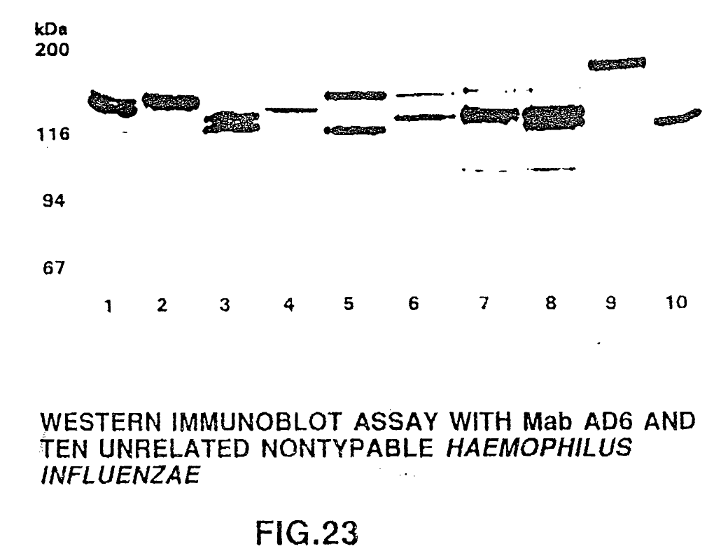

Having identified the approximate locations of the epitopes on HMW1 and HMW2 recognized by the two monoclonals, the extent to which these epitopes were shared by the high molecular weight proteins of heterologous nontypable Haemophilus strains was next determined. When examined in Western blot assays with bacterial cell sonicates, Mab AD6 was reactive with epitopes expressed on the high molecular weight proteins of 75% of the inventor's collection of more than 125 nontypable Haemophilus influenzae strains. In fact, this monoclonal appeared to recognize epitopes expressed on high molecular weight proteins in virtually all nontypable Haemophilus strains which we previously identified as expressing HMW1/HMW2-like proteins. FIG. 23 is an example of a Western blot demonstrating the reactivity of Mab AD6 with a representative panel of such heterologous strains. As can be seen, the monoclonal antibody recognizes one or two bands in the 100 to 150 kDa range in each of these strains. For reference, the strain shown in lane 1 is prototype strain 12 and the two bands visualized represent HMW1 and HMW2 as the upper and lower immunoreactive bands, respectively.

In contrast to the broad cross-reactivity observed with Mab AD6, Mab 10C5 was much more limited in its ability to recognize high molecular weight proteins in heterologous strains. Mab 10C5 recognized high molecular weight proteins in approximately 40% of the strains which expressed HMW1/HMW2-like proteins. As was the case with Mab AD6, Mab 10C5 did not recognize proteins in any the nontypable Haemophilus strains which did not express HMW1/HMW2-like proteins.

In a limited fashion, the reactivity of Mab AD6 with surface-exposed epitopes on the heterologous strains has been examined. In the bottom two panels of FIG. 20 are electron micrographs demonstrating the reactivity of Mab AD6 with surface-accessible epitopes on nontypable Haemophilus strains 5 and 15. As can be seen, abundant colloidal-gold particles are evident on the surfaces of each of these strains, confirming their surface expression of the AD6 epitope. Although limited in scope, these data suggest that the AD6 epitope may be a common surface-accessible epitope on the high molecular weight adhesion proteins of most nontypable Haemophilus influenzae which express HMW1/HMW2-like proteins.

### SUMMARY OF DISCLOSURE

In summary of this disclosure, the present invention provides high molecular weight proteins of non-typeable Haemophilus, genes coding for the same and vaccines incorporating such proteins. Modifications are possible within the scope of this invention.

TABLE 1
Effect of mutation of high molecular weight
proteins on adherence to Chang epithelial cells by
nontypable H. influenzae.

ADHERENCE %*

| | | Relative to |
|---|---|---|
| Strain | % Inoculation | wild Type† |
| Strain 12 derivatives | 87.76 ± 5.9 | 100.0 ± 6.7 |
| wild type | | |
| HMW1⁻ mutant | 6.0 ± 0.9 | 6.8 ± 1.0 |
| HMW2⁻ mutant | 89.9 ± 10.8 | 102.5 ± 12.3 |
| HMW1⁻/HMW2⁻ mutant | 2.0 ± 0.3 | 2.3 ± 0.3 |
| Strain 5 derivatives | 78.7 ± 3.2 | 100.0 ± 4.1 |
| wild type | | |
| HMW1-like mutant | 15.7 ± 2.6 | 19.9 ± 3.3 |
| HMW2-like mutant | 103.7 ± 14.0 | 131.7 ± 17.8 |
| double mutant | 3.5 ± 0.6 | 4.4 ± 0.8 |

*Numbers represent mean (± standard error of the mean) of measurements in triplicate or quadruplicate from representative experiments.
†Adherence values for strain 12 derivatives are relative to strain 12 wild type; values for strain 5 derivatives are relative to strain 5 wild type.

TABLE 2
Adherence by E. coli DH5α and HB101 harboring
hmw1 or hmw2 gene clusters.

| | Adherence relative to H. |
|---|---|
| Strain* | influenzae strain 12† |
| DH5α (pT7-7) | 0.7 ± 0.02 |
| DH5α (pHMW1-14) | 114.2 ± 15.9 |
| DH5α (pHMW2-21) | 14.0 ± 3.7 |
| HB101 (pT7-7) | 1.2 ± 0.5 |
| HB101 (pHMW1-14) | 93.6 ± 15.8 |
| HB101 (pHMW2-21) | 3.6 ± 0.9 |

*The plasmid pHMW1-14 contains the hmw1 gene cluster, while pHMW2-21 contains the hmw2 gene cluster; pT7-7 is the cloning vector used in these constructs.
†Numbers represent the mean (± standard error of the mean) of measurements made in triplicate from representative experiments.

TABLE 3
Protective ability of HMW protein against non-
typeable H. influenzae challenge in chinchilla model

| | | | Number of Animals Showed | | |
|---|---|---|---|---|---|
| | | | Positive Ear Infection | | |
| | | | | | cfu of |
| Group | | Total | | Otoscopic | Bacteria/ |
| (#) | Antigens | Animals | Tympanogram | Examination | 10 μL |
| 1 | HMW | 5 | 0 | 0 | 0 |
| 2 | None | 5 | 5 | 5 | 850-3200 |
| | | | | | (4/5) |
| 3 | Conva- | 4 | 0 | 0 | 0 |
| | lescent | | | | |

1-20. (cancelled)

21. An isolated and purified nucleic acid molecule encoding a high molecular weight protein (HMW) of a non-typeable Haemophilus strain.

22. The nucleic acid molecule of claim 21 encoding protein HMW1, HMW2, HMW3 or HMW4 or a variant or fragment of said protein retaining the immunological ability to protect against disease caused by a non-typeable Haemophilus strain.

23. The nucleic acid molecule of claim 22 having the DNA sequence shown in FIG. 1 (SEQ ID No: 1) and encoding protein HMW1 having the derived amino acid sequence of FIG. 2 (SEQ ID No: 2).

24. The nucleic acid molecule of claim 22 having the DNA sequence shown in FIG. 3 (SEQ ID No: 3) and encoding protein HMW2 having the derived amino acid sequence of FIG. 4 (SEQ ID No: 4).

25. The nucleic acid molecule claimed in claim 22 having the DNA sequence shown in FIG. 8 (SEQ ID No: 7) and encoding protein HMW3 having the derived amino acid sequence of FIG. 10 (SEQ ID No: 9).

26. The nucleic acid molecule claimed in claim 22 having the DNA sequence shown in FIG. 9 (SEQ ID No: 8) and encoding protein HMW4 having the derived amino acid sequence of FIG. 10 (SEQ ID No: 10).

27. An isolated and purified nucleic acid molecule encoding a high molecular weight protein (HMW) of a non-typeable Haemophilus strain, which comprise:
(a) a DNA sequence as shown in any one of FIGS. 1, 3, 8 and 9 (SEQ ID Nos: 1, 3, 7 and 8);
(b) a DNA sequence encoding an amino acid sequence as shown in any one of FIGS. 2, 4 and 10 (SEQ ID Nos: 2, 4, 9 and 10); or
(c) a DNA sequence which hybridizes under stringent conditions to any one of the DNA sequences of (a) and (b).

28. The nucleic acid molecule of claim 27 wherein the DNA sequence (c) have at least about a 90% identity of sequence to the DNA sequences (a) or (b).

29. A purified and isolated gene cluster comprising a nucleotide sequence for a structural gene encoding a high molecular weight protein of a non-typeable Haemophilus strain and at least one downstream nucleotide sequence for an accessory gene for effecting expression of a gene product fully encoded by said structural gene.

30. The gene cluster claimed in claim 29 comprising a DNA sequence coding for protein HMW1 or HMW2 and two downstream accessory genes.

31. The gene cluster of claim 30 having the DNA sequence shown in FIG. 6 (SEQ ID No: 5).

32. The gene cluster of claim 30 having the DNA sequence shown in FIG. 7 (SEQ ID No: 6).

33. A vector for transformation of a host comprising the nucleic acid molecule of claim 21 or 27.

34. An expression vector adapted for transformation of a host comprising the gene cluster of claim 29 and expression means operatively coupled to the gene cluster for expression by the host of the high molecular weight protein of a non-typeable Haemophilus strain.

35. A transformed host containing an expression vector as claimed in claim 34.

36. A recombinant high molecular weight (HMW) protein of a non-typeable Haemophilus strain producible by the transformed host of claim 35.

37. An isolated and purified high molecular weight (HMW) protein of non-typeable Haemophilus which is encoded by a nucleic acid molecule as defined in claim 21, or any variant or fragment thereof retaining the immunological ability to protect against disease caused by a non-typeable Haemophilus strain.

38. The protein of claim 37 which is characterized by at least one surface-exposed B-cell epitope which is recognized by monoclonal antibody AD6.

39. The protein of claim 37 which is HMW1 encoded by the DNA sequence shown in FIG. 1 (SEQ ID No: 1), having the derived amino acid sequence of FIG. 2 (SEQ ID No: 2) and having an apparent molecular weight of 125 kDa.

40. The protein claim 37 which is HMW2 encoded by the DNA sequence shown in FIG. 3 (SEQ ID No: 3) and having the derived amino acid sequence of FIG. 4 (SEQ ID No: 4) and having an apparent molecular weight of 120 kDa.

41. The protein claimed in claim 37 which is HMW3 encoded by the DNA sequence shown in FIG. 8 (SEQ ID No: 7) and having the derived amino acid sequence of FIG. 10 (SEQ ID No: 9) and having an apparent molecular weight of 125 kDa.

42. The protein claimed in claim 37 which is HMW4 encoded by the DNA sequence shown in FIG. 9 (SEQ ID No: 8) and having the derived amino acid sequence shown in FIG. 10 (SEQ ID No: 10) and having an apparent molecular weight of 123 kDa.

43. An isolated and purified high molecular weight (HMW) protein of non-typeable Haemophilus influenzae which is antigenically related to the filamentous hemagglutinin surface protein of Bordetella pertussis.

44. The protein of claim 43 which is HMW1, HMW2, HMW3 or HMW4.

45. A conjugate comprising a protein as claimed in claim 37 or 43 linked to an antigen, hapten or polysaccharide for eliciting an immune response to said antigen, hapten or polysaccharide.

46. The conjugate as claimed in claim 45 wherein said polysaccharide is a protective polysaccharide against Haemophilus influenzae type b.

47. A synthetic peptide having an amino acid sequence containing at least six amino acids and no more than 150 amino acids and corresponding to at least one protective epitope of a high molecular weight protein of non-typeable Haemophilus influenzae.

48. The peptide of claim 47 wherein said protein is HMW1, HMW2, HMW3 or HMW4.

49. The peptide of claim 47 wherein the epitope is recognized by at least one of monoclonal antibodies AD6 and 10C5.

50. The peptide as claimed in claim 48 wherein the epitope is located within 75 amino acids of the carboxy terminus of the HMW1 or HMW2 protein is recognized by monoclonal antibody AD6.

51. Monoclonal antibody AD6 (ATCC ______).

52. Monoclonal antibody 10C5 (ATCC ______).
